(19) United States
(12) Patent Application Publication
(10) Pub. No.: US 2025/0082679 A1
(43) Pub. Date: Mar. 13, 2025

(54) Method of Targeting Cells and Associated Compositions

(71) Applicant: Thunder Biotech, Inc., Provo, UT (US)

(72) Inventors: Kim Leslie O'Neill, Provo, UT (US); Michelle Townsend, Provo, UT (US)

(21) Appl. No.: 18/573,239

(22) PCT Filed: Jun. 24, 2022
(86) PCT No.: PCT/US2022/034940

§ 371 Date: Dec. 21, 2023

Related U.S. Application Data
(60) Provisional application No. 63/214,544, filed on Jun. 24, 2021.

(51) Int. Cl. A61K 35/15; A61K 39/00; A61K 48/00; A61P 35/00; C07K 14/725; C07K 16/40; C12N 5/0784; C12N 5/0786; C12N 15/113; C12N 15/88

(52) U.S. Cl. CPC A61K 35/15; A61K 40/17; A61K 40/31; A61K 40/4255; A61K 48/005; A61P 35/00; C07K 14/7051; C07K 16/40; C12N 5/0639; C12N 5/0645; C12N 15/113; C12N 15/88; A61K 2239/13; A61K 2239/17; A61K 2239/21; C07K 2317/53; C07K 2317/569; C07K 2317/622; C07K 2319/03; C12N 2310/14; C12N 2510/00

(57) ABSTRACT

Described herein is a delivery system that targets leukocytes and in some embodiments monocytes to deliver nucleic acid encoding a chimeric antigen receptor. Additionally, described herein is a method of using a delivery system to transduce leukocytes and in some embodiments monocytes to deliver nucleic acid encoding a chimeric antigen receptor. The delivery system may also function to activate the target cell by providing a ligand to the chimeric antigen receptor. Furthermore, described herein is a delivery system that also include a nucleic acid inhibitor that decreases the expression of a protein that forms a part of the pathway that degrades the chimeric receptor. In embodiments, the binding of a ligand to the extracellular domain of the chimeric receptor activates the intracellular portion of the chimeric receptor. Activation of the intracellular portion of the chimeric receptor may polarize the macrophage into an M1 or M2 macrophage.

### CROSS REFERENCE TO RELATED APPLICATIONS

This application is a national phase entry under 35 U.S.C. § 371 of International Patent Application PCT/US2022/034940, filed Jun. 24, 2022, designating the United States of America and published in English as International Patent Publication WO 2022/272088 on Dec. 29, 2022, which claims the benefit under Article 8 of the Patent Cooperation Treaty to U.S. Patent Application Ser. No. 63/214,544, filed Jun. 24, 2021, the entireties of which are hereby incorporated by reference.

### TECHNICAL FIELD

The present disclosure relates generally to biotechnology. More specifically, the present disclosure relates methods of targeting cells. The methods include targeting cells to provide a molecule of interest. Examples of such molecules are chimeric antigen receptors and nucleic acids encoding chimeric antigen receptors. Cells include leukocytes harboring nucleic acids encoding chimeric antigen receptors with or without expression of the chimeric antigen receptors, and associated methods. In specific embodiments, the present disclosure relates to targeting leukocytes with a delivery system containing nucleic acids encoding chimeric antigen receptors. More specifically, the present disclosure relates to targeting monocytes, or any myeloid cell, such as macrophages or dendritic cells.

### BACKGROUND

Cancer consists of a group of diseases which involve unregulated cell growth and death, genome instability and mutations, tumor-promoting inflammation, induction of angiogenesis, immune system evasion, deregulation of metabolic pathways, immortal cell replication, and metastatic tissue invasion [1]. Cancer is the second leading cause of death in the United States after heart disease [2]. More than 1.6 million new cases of cancer are projected to be diagnosed each year, with more than 580,000 Americans expected to die (about 1600 cancer deaths per day), accounting for nearly 1 in 4 of all American deaths [2,3].

The immune system plays an important role in the development and progression of cancer. Immune cell infiltration to the tumor site can adversely affect malignancy progression and metastasis [4, 5]. Infiltration of macrophages into the tumor site has been shown to account for more than 50% of the tumor mass in certain breast cancer cases suggesting macrophages have a significant role in tumor progression [6-8].

Macrophages are cells derived from the myeloid lineage and belong to the innate immune system. They are derived from blood monocytes that migrate into tissue. Macrophages and other phagocytes are leukocytic cells capable of phagocytosing or taking up bacteria, cellular debris, and particles through energy-consuming membrane-engulfing as a characteristic phenotype. Their primary role is early response to foreign material contamination and its clearance. Macrophages have been known to uptake foreign materials within a matter of minutes, increasing their rates of phagocytosis for positively charged and bacteria-specific proteins. They also play an important role in both the initiation and resolution of inflammation [9, 10]. Moreover, macrophages can display different responses, ranging from pro-inflammatory to anti-inflammatory, depending on the type of stimuli they receive from the surrounding microenvironment [11]. Two major macrophage phenotypes have been proposed which correlate with extreme macrophage responses: M1 and M2.

M1 pro-inflammatory macrophages are activated upon contact with certain molecules such as lipopolysaccharide (LPS), IFN-γ, IL-1β, TNF-α, and Toll-like receptor engagement. M1 macrophages constitute a potent arm of the immune system deployed to fight infections. They are capable of either direct (pathogen pattern recognition receptors) or indirect (Fe receptors, complement receptors) recognition of the pathogen. They are also armed in their ability to produce reactive oxygen species (ROS) as means to help kill pathogens. In addition, M1 macrophages secrete pro-inflammatory cytokines and chemokines that attract other types of immune cells and integrating/orchestrate the immune response. M1 activation is induced by IFN-g, TNFa, GM-CSF, LPS and other toll-like receptors (TLR) ligands.

In contrast, M2 anti-inflammatory macrophages, also known as alternatively activated macrophages, are activated by anti-inflammatory molecules such as IL-4, IL-13, and IL-10 [12, 13]. M2 macrophages exhibit immunomodulatory, tissue repair, and angiogenic properties which allow them to recruit regulatory T cells to sites of inflammation. M2 macrophages do not constitute a uniform population and often are further subdivided into M2a, M2b, M2c and M2d phenotypes. The common denominator of all three subpopulations is high IL-10 production accompanied by low production of IL-12. One of their signatures is production of Arginase-1 an enzyme that depletes L-arginine thereby suppressing T cell responses and depriving iNOS of its substrate.

The in vivo molecular mechanisms of macrophage polarization are poorly characterized because of the variety of signals macrophages experience in the cellular microenvironment [10, 14]. In recent years, progress has been made in identifying in vivo macrophage polarization under physiological conditions such as ontogenesis, pregnancy, and pathological conditions such as allergies, chronic inflammation, and cancer. It is understood that in vitro macrophage polarization is plastic and macrophages, with the help of cytokines, can be polarized back and forth to either phenotype [15, 16]. Interferon gamma (IFN-γ) and IL-4 are two cytokines that can polarize macrophages to M1 and M2 phenotypes, respectively [15].

The presence of macrophages is crucial for tumor progression and growth, and has implications in determining prognosis [17, 18]. Because macrophages can exhibit both pro-inflammatory and anti-inflammatory properties, it is important to understand their polarization and function in tumor progression and metastasis.

### Macrophage Polarization

The tumor microenvironment can affect macrophage polarization. The process of polarization can be diverse and complex because of the hostile environment of IL-10, glucocorticoid hormones, apoptotic cells, and immune complexes that can interfere with innate immune cells function [11, 19]. The mechanisms of polarization are still unclear but we know they involve transcriptional regulation. For example, macrophages exposed to LPS or IFN-γ will polarize towards an M1 phenotype, whereas macrophages exposed to IL-4 or IL-13 will polarize towards an M2 phenotype. LPS can interact with Toll-like receptor 4 (TLR4) on the surface of macrophages inducing the Trif and MyD88 pathways, inducing the activation of transcription factors IRF3, AP-1, and NFκB and thus activating TNFs genes, interferon genes, CXCL10, NOS2, IL-12, etc. which are necessary in a pro-inflammatory M1 macrophage response [20]. Similarly, IL-4 and IL-13 bind to IL-4R, activation the Jak/Stat6 pathway, which regulates the expression of CCL17, ARG1, IRF4, IL-10, SOCS3, etc., which are genes associated with an anti-inflammatory response (M2 response).

Additional mechanisms of macrophage polarization include microRNA (miRNA) micromanagement. It is known that miRNAs are small non-coding RNA of 22 nucleotides in length that regulate gene expression post-transcriptionally, as they affect the rate of mRNA degradation. Several miRNAs have been shown to be highly expressed in polarized macrophages, especially miRNA-155, miRNA-125, miRNA-378 (M1 polarization), and miRNA let-7c, miRNA-9, miRNA-21, miRNA-146, miRNA147, miRNA-187 (M2 polarization) [21].

Macrophage polarization is a complex process, where macrophages behave and elicit different responses depending on microenvironment stimuli. Therefore, macrophage polarization is better represented by a continuum of activation states where M1 and M2 phenotypes are the extremes of the spectrum. In recent years, there has been controversy regarding the description of macrophage activation and macrophage polarization. A recent paper published by Murray et al. describes a set of standards to be considered for the consensus description of macrophage activation, polarization, activators, and markers. This publication was much needed for the definition and characterization of activated and thus polarized macrophages [22].

### M1 Phenotype

M1 pro-inflammatory macrophages or classically activated macrophages are aggressive, highly phagocytic, and produce large amounts of reactive oxygen and nitrogen species, thereby promoting a Th1 response [11]. M1 macrophages secrete high levels of two important inflammatory cytokines, IL-12 and IL-23. IL-12 induces the activation and clonal expansion of Th17 cells, which secrete high amounts of IL-17, which contributes to inflammation [23]. These characteristics allow M1 macrophages to control metastasis, suppress tumor growth, and control microbial infections [24]. Moreover, the infiltration and recruitment of M1 macrophages to tumor sites correlates with a better prognosis and higher overall survival rates in patients with solid tumors [17, 18, 25-28].

Polarization of macrophages to the M1 phenotype is regulated in vitro by inflammatory signals such as IFN-γ. TNF-α, IL-1B and LPS as well as transcription factors and miRNAs [29, 30]. Classically activated macrophages initiate the induction of the STAT1 transcription factor which targets CXCL9, CXCL10 (also known as IP-10), IFN regulatory factor-1, and suppressor of cytokine signaling-1 [31]. Cytokine signaling-1 protein functions downstream of cytokine receptors, and takes part in a negative feedback loop to attenuate cytokine signaling. In the tumor microenvironment, Notch signaling plays an important role in the polarization of M1 macrophages, as it allows transcription factor RBP-J to regulate classical activation. Macrophages that are deficient in Notch signaling express an M2 phenotype regardless of other extrinsic inducers [32]. One crucial miRNA, miRNA-155, is upregulated when macrophages are transitioning from M2 to M1. The M1 macrophages overexpressing miRNA-155 are generally more aggressive and are associated with tumor reduction [33]. Moreover, miRNA-342-5p has been found to foster a greater inflammatory response in macrophages by targeting Akt1 in mice. This miRNA also promotes the upregulation of Nos2 and IL-6, both of which act as inflammatory signals for macrophages [34]. Other miRNAs such as miRNA-125 and miRNA-378 have also been shown to be involved in the classical activation pathway of macrophages (M1) [35]. M1 transcription factors include STAT1, C/EBP-α, C/EBP-8, IRF9, KLF6, NF-kB, AP1, HIF1α.

Classically activated macrophages are thought to play an important role in the recognition and destruction of cancer cells as their presence usually indicates good prognosis. After recognition, malignant cells can be destroyed by M1 macrophages through several mechanisms, which include contact-dependent phagocytosis and cytotoxicity (i.e. cytokine release such as TNF-α) [24]. Environmental signals such as the tumor microenvironment or tissue-resident cells, however, can polarize M1 macrophages to M2 macrophages. In vivo studies of murine macrophages have shown that macrophages are plastic in their cytokine and surface marker expression and that re-polarizing macrophages to an M1 phenotype in the presence of cancer can help the immune system reject tumors [19].

### M2 Phenotype

M2 macrophages are anti-inflammatory and aid in the process of angiogenesis and tissue repair. They express scavenger receptors and produce large quantities of IL-10 and other anti-inflammatory cytokines [33, 36]. Expression of IL-10 by M2 macrophages promotes a Th2 response. Th2 cells consequently upregulate the production of IL-3 and IL-4. IL-3 stimulates proliferation of all cells in the myeloid lineage (granulocytes, monocytes, and dendritic cells), in conjunction with other cytokines, e.g., Erythropoietin (EPO), Granulocyte macrophage colony-stimulating factor (GM-CSF), and IL-6. IL-4 is an important cytokine in the healing process because it contributes to the production of the extracellular matrix [23]. M2 macrophages exhibit functions that may help tumor progression by allowing blood vessels to feed the malignant cells and thus promoting their growth. The presence of macrophages (thought to be M2) in most solid tumors negatively correlates with treatment success and longer survival rates [37]. Additionally, the presence of M2 macrophages has been linked to the metastatic potential in breast cancer. In a recent paper, Lin et al. found that early recruitment of macrophages to the breast tumor sites in mice increase angiogenesis and incidence of malignancy [38]. It is thought that the tumor microenvironment helps macrophages maintain an M2 phenotype [23, 39]. Anti-inflammatory signals present in the tumor microenvironment such as adiponectin and IL-10 can enhance an M2 response [41]. M2 transcription factors include PPARγ, STAT3, STAT6, C/EBP-β, IRF4, KLF4, GATA3, c-MYC.

### Tumor Associated Macrophages (TAMs)

Cells exposed to a tumor microenvironment behave differently. For example, tumor associated macrophages found in the periphery of solid tumors are thought to help promote tumor growth and metastasis and have an M2-like phenotype [42]. Tumor associated macrophages can be cither tissue resident macrophages or recruited macrophages derived from the bone marrow (macrophages that differentiate from monocytes to macrophages and migrate into tissue). A study by Cortez-Retamozo found that high numbers of TAM precursors in the spleen migrate to the tumor stroma, suggesting this organ as a TAM reservoir also [43]. TAM precursors found in the spleen were found to initiate migration through their CCR2 chemokine receptor [43]. Recent studies have found CSF-1 as the primary factor that attracts macrophages to the tumor periphery, and that CSF-1 production by cancer cells predicts lower survival rates and it indicates an overall poor prognosis [44-46]. Other cytokines such as TNF-α and IL-6 have been also linked to the accumulation/recruitment of macrophages to the tumor periphery [45].

It is thought that macrophages that are recruited around the tumor borders are regulated by an “angiogenic switch” that is activated in the tumor. The angiogenic switch is defined as the process by which the tumor develops a high-density network of blood vessels that potentially allows the tumor to become metastatic and is necessary for malignant transition. In a breast cancer mouse model, it was observed that the presence of macrophages was required for a full angiogenic switch. When macrophage maturation, migration, and accumulation around the tumor was delayed, the angiogenic switch was also delayed suggesting that the angiogenic switch does not occur in the absence of macrophages and that macrophage presence is necessary for malignancy progression [47]. Moreover, the tumor stromal cells produce chemokines such as CSF1, CCL2, CCL3, CCL5, and placental growth factor that will recruit macrophages to the tumor surroundings. These chemokines provide an environment for macrophages to activate the angiogenic switch, in which macrophages will produce high levels of IL-10, TGF-β, ARG-1 and low levels of IL-12, TNF-α, and IL-6. The level of expression of these cytokines suggests macrophages modulate immune evasion. It is important to note that macrophages are attracted to hypoxic tumor environments and will respond by producing hypoxia-inducible factor-1α (HIF-1α) and HIF-2α, which regulate the transcription of genes associated with angiogenesis. During the angiogenic switch, macrophages can also secrete VEGF (stimulated by the NF-κB pathway), which will promote blood vessel maturation and vascular permeability [48].

Tumor associated macrophages are thought to be able to maintain their M2-like phenotype by receiving polarization signals from malignant cells such as IL-1R and MyD88, which are mediated through IkB kinase β and NF-kB signaling cascade. Inhibition of NF-kB in TAMs promotes classical activation [40]. Moreover, another study suggested that p50 NF-kB subunit was involved in suppression of M1 macrophages, and reduction of inflammation promoted tumor growth. A p50 NF-kB knock-out mouse generated by Saccani et. al suggested that M1 aggressiveness was restored upon p50 NF-kB knockout, reducing tumor survival [49].

Because the tumor mass contains a great number of M2-like macrophages, TAMs can be used as a target for cancer treatment. Reducing the number of TAMs or polarizing them towards an M1 phenotype can help destroy cancer cells and impair tumor growth [50-52]. Luo and colleagues used a vaccine against legumain, a cysteine protease and stress protein upregulated in TAMs thought to be a potential tumor target [52]. When the vaccine against legumain was administered to mice, genes controlling angiogenesis were downregulated and tumor growth was halted [52].

### Metabolism and Activation Pathways

Metabolic alterations present in tumor cells are controlled by the same genetic mutations that produce cancer [53]. As a result of these metabolic alterations, cancer cells can produce signals that can modify the polarization of macrophages and promote tumor growth [54, 55].

M1 and M2 macrophages demonstrate distinct metabolic patterns that reflect their dissimilar behaviors [56]. The M1 phenotype increases glycolysis and skews glucose metabolism towards the oxidative pentose phosphate pathway, thereby decreasing oxygen consumption and consequently producing large amounts of radical oxygen and nitrogen species as well as inflammatory cytokines such as TNF-α, IL-12, and IL-6 [56, 57]. The M2 phenotype increases fatty acid intake and oxidation, which decreases flux towards the pentose phosphate pathway while increasing the overall cell redox potential, consequently upregulating scavenger receptors and immunomodulatory cytokines such as IL-10 and TGF-β [56].

Multiple metabolic pathways play important roles in macrophage polarization. Protein kinases, such as Akt1 and Akt2, alter macrophage polarization by allowing cancer cells to survive, proliferate, and use an intermediary metabolism [58]. Other protein kinases can direct macrophage polarization through glucose metabolism by increasing glycolysis and decreasing oxygen consumption [57, 59]. Shu and colleagues were the first to visualize macrophage metabolism and immune response in vivo using a PET scan and a glucose analog [60].

L-arginine metabolism also exhibits discrete shifts important to cytokine expression in macrophages and exemplifies distinct metabolic pathways which alter TAM-tumor cell interactions [61]. Classically activated (M1) macrophages favor inducible nitric oxide synthase (iNOS). The iNOS pathway produces cytotoxic nitric oxide (NO), and consequently exhibits anti-tumor behavior. Alternatively activated (M2) macrophages have been shown to favor the arginase pathway and produce ureum and l-ornithine, which contribute to progressive tumor cell growth [61, 62].

Direct manipulation of metabolic pathways can alter macrophage polarization. The carbohydrate kinase-like protein (CARKL) protein, which plays a role in glucose metabolism, has been used to alter macrophage cytokine signatures [56, 57]. When CARKL is knocked down by RNAi, macrophages tend to adopt an M1-like metabolic pathway (metabolism skewed towards glycolysis and decreased oxygen consumption). When CARKL is overexpressed, macrophages adopt an M2-like metabolism (decreased glycolytic flux and more oxygen consumption) [56]. When macrophages adopt an M1-like metabolic state through LPS/TLR4 engagement, CARKL levels decrease, genes controlled by the NFκB pathway are activated (TNF-α, IL-12, and IL-6), and cell redox potential increases due to growing concentrations of NADH:NAD+ and GSH:GSSSG complexes. During an M2-like metabolic state, macrophages upregulate CARKL and genes regulated by STAT6/IL-4 (IL-10 and TGF-β).

Obesity can also affect macrophage polarization. Obesity is associated with a state of chronic inflammation, an environment that drives the IL4/STAT6 pathway to activate NKT cells, which drive macrophages towards an M2 response. During late-stage diet-induced obesity, macrophages migrate to adipose tissue, where immune cells alter levels of T_{H}1 or T_{H}2 cytokine expression in the adipose tissue, causing an M2 phenotype bias and possibly increased insulin sensitivity [63].

M1 phenotype bias by targeting metabolic pathways in TAMS may offer an alternative means of reducing tumor growth and metastasis.

### Macrophage Immunotherapy Approaches Against Cancer

The role of cancer immunotherapy is to stimulate the immune system to recognize, reject, and destroy cancer cells. Cancer immunotherapy with monocytes and specifically macrophages has the goal to polarize macrophages towards a pro-inflammatory response (M1), thus allowing the macrophages and other immune cells to destroy the tumor. Many cytokines and bacterial compounds can achieve this in vitro, although the side effects are typically too severe in vivo. The key is to find a compound with minimal or easily managed patient side effects. Immunotherapy using monocytes/macrophages has been used in past decades and new approaches are being developed every year [64, 65]. Early immunotherapy has established a good foundation for better cancer therapies and increased survival rate in patients treated with immunotherapies [66].

Some approaches to cancer immunotherapy include the use of cytokines or chemokines to recruit activated macrophages and other immune cells to the tumor site which allow for recognition and targeted destruction of the tumor site [67, 68]. IFN-α and IFN-β have been shown to inhibit tumor progression by inducing cell differentiation and apoptosis [69]. Also, IFN treatments are anti-proliferative and can increase S phase time in the cell cycle [70, 71]. Zhang and colleagues performed a study in nude mice using IFN-β gene therapy to target human prostate cancer cells. Their results indicate that adenoviral-delivered IFN-β gene therapy involves macrophages and helps suppress growth and metastasis [72].

The macrophage inhibitory factor (MIF) is another cytokine that can be used in cancer immunotherapy. MIF is usually found in solid tumors and indicates poor prognosis. MIF inhibits aggressive macrophage function and drives macrophages toward an M2 phenotype, which can aid tumor growth and progression. Simpson, Templeton & Cross (2012) found that MIF induces differentiation of myeloid cells, macrophage precursors, into a suppressive population of myeloid cells that express an M2 phenotype [73]. By targeting MIF, they were able to deplete this suppressive population of macrophages, inhibiting their growth and thus control tumor growth and metastasis [73].

The chemokine receptor type 2. CCR2, is crucial to the recruitment of monocytes to inflammatory sites and it has been shown as a target to prevent the recruitment of macrophages to the tumor site, angiogenesis, and metastasis. Sanford and colleagues (2013) studied a novel CCR2 inhibitor (PF-04136309) in a pancreatic mouse model, demonstrating that the CCR2 inhibitor depleted monocyte/macrophage recruitment to the tumor site, decreased tumor growth and metastasis, and increased antitumor immunity [74]. Another recent study by Schmall et al. showed that macrophages co-cultured with 10 different human lung cancers upregulated CCR2 expression. Moreover, they showed that tumor growth and metastatis were reduced in a lung mouse model treated with a CCR2 antagonist [75].

Other studies have used liposomes to deliver drugs to deplete M2 macrophages from tumors and to stop angiogenesis. Cancer cells that express high levels of IL-1β grow faster and induce more angiogenesis in vivo. Kimura and colleagues found that macrophages exposed to tumor cells expressing IL-1β produced higher levels of angiogenic factors and chemokines such as vascular endothelial growth factor A (VEG-A), IL-8, monocyte chemoattractant protein 1, etc., facilitating tumor growth and angiogenesis [76]. When they used clodronate liposomes to deplete macrophages, they found fewer IL-1β-producing tumor cells. They also found that by inhibiting NF-kB and AP-1 transcription factors in the cancer cells, tumor growth and angiogenesis were reduced. These findings may suggest that macrophages that surround the tumor site may be involved in promoting tumor growth and angiogenesis [76].

Compounds such as methionine enkephalin (MENK) have anti-tumor properties in vivo and in vitro. MENK can polarize M2 macrophages to M1 macrophages by downregulating CD206 and arginase-1 (M2 markers) while upregulating CD64, MHC-II, and the production of nitric oxide (M1 markers). MENK can also upregulate TNF-α and downregulate IL-10 [77].

Recent studies have focused on bisphosphonates as a potential inhibitor of M2 macrophages. Bisphosphonates are commonly used to treat metastatic breast cancer patients to prevent skeletal complications such as bone resorption [78]. While bisphosphonates stay in the body for short periods of time, bisphosphonates can target osteoclasts, cells in the same family as macrophages, due to their high affinity for hydroxyapatite. Once bisphosphonates bind to the bones, the bone matrix internalizes the bisphosphonates by endocytosis. Once in the cytoplasm, bisphosphonates can inhibit protein prenylation, an event that prevents integrin signaling and endosomal trafficking, thereby forcing the cell to go apoptotic [69]. Until recently, it was unknown whether bisphosphonates could target tumor associated macrophages but a recent study by Junankar et al. has shown that macrophages uptake nitrogen-containing bisphosphonate compounds by pinocytosis and phagocytosis, an event that does not occur in epithelial cells surrounding the tumor [79]. Forcing TAMs to go apoptotic using bisphosphonates could reduce angiogenesis and metastasis.

Additional approaches to cancer immunotherapy include the use of biomaterials that may elicit an immune response. Cationic polymers are used in immunotherapy because of their reactivity once dissolved in water. Chen et al. used cationic polymers including PEI, polylysine, cationic dextran and cationic gelatin to produce a strong Th1 immune response [77]. They were also able to induce proliferation of CD4+ cells and secretion of IL-12 typical of M1 macrophages [77]. Huang and colleagues also used biomaterials to trigger TAMs to produce an anti-tumor response by

targeting TLR4 [80]. This study found that TAMs were able to polarize to an M1 phenotype and express IL-12. They found that these cationic molecules have direct tumoricidal activity and demonstrate tumor reduction in mice [80].

### TLR4

Toll-like receptor 4 is a protein in humans that is encoded by the TLR4 gene. TLR 4 detects lipopolysaccharide (LPS) on gram negative bacteria and thus plays a fundamental role in the recognition of danger and the activation of the innate immune system (FIG. 7). It cooperates with LY96 (MD-2) and CD14 to mediate signal transduction when macrophages are induced by LPS. The cytoplasmic domain of TLR4 is responsible for the activation of M1 macrophages when they detect the presence of LPS. This is the functional portion of the receptor that would be coupled to the MOTO-CAR (i.e. chimeric receptor) to induce activation of a monocyte and specifically a macrophage when the CAR binds its target protein.

The adaptor proteins MyD88 and TIRAP contribute to the activation of several and possibly all pathways via direct interactions with TLR4's Toll/interleukin-1 receptor (IL-1R) (TIR) domain. However, additional adaptors that are required for the activation of specific subsets of pathways may exist, which could contribute to the differential regulation of target genes.

### BRIEF SUMMARY

This disclosure provides delivery systems that target cells to deliver nucleic acid encoding chimeric receptors. In embodiments, the cells are leukocytes. In particular embodiments the cells are monocytes, macrophages, or dendritic cells. In embodiments, the delivery system comprises an activating agent that activates the leukocyte, for example a monocyte, and, in embodiments, polarizes a macrophage or other dendritic cell into either an M1 or M2 macrophage.

This disclosure provides a method of using a delivery system that target cells to deliver a nucleic acid. In embodiments, the nucleic acid encodes a protein of interest, for example, but not limited to, a chimeric antigen receptor. In certain embodiments, the nucleic acid is DNA, RNA, an artificial nucleic acid (such as, for example, PNAs, morpholino, locked nucleic acids, glycol nucleic acids, and threose nucleic acids), or a combination thereof. In a specific embodiment, the nucleic acid is an mRNA. In some embodiments, the cells are leukocytes. In particular embodiments the cells are monocytes, and in some embodiments the cells are macrophages or dendritic cells. In some embodiments the method of this disclosure provides a delivery system that comprises an activating agent that activates a leukocyte, in some embodiments a monocyte, and, by way of non-limiting example, can polarize a macrophage into cither M1 or M2 macrophages. This disclosure provides chimeric antigen receptors (CARs) that include a cytoplasmic domain, a transmembrane domain, and an extracellular domain. In some embodiments, the cell, by way of non-limiting example a leukocyte, in some embodiments a monocyte, in some embodiments a macrophage or dendritic cell will be transduced by the delivery system with nucleic acid encoding a CAR. In some embodiments the delivery system will also include a nucleic acid inhibitor that decreases the expression of a protein that forms part of the pathway that targets the CAR for degradation and/or degrades the CAR in a mammalian cell. In further embodiments the activator may be a ligand for the extracellular domain of the CAR that when bound to the CAR will activate the cytoplasmic domain, and in some embodiments polarize a macrophage into an M1 or M2 macrophage.

In embodiments, the cytoplasmic domain includes a cytoplasmic portion of a receptor that can activate the cell. In some embodiments, this activation will be an activation of a leukocyte, and in some embodiments a monocyte, and in particular embodiments will polarize a macrophage or dendritic cell. In further embodiments, a wild-type protein comprising the cytoplasmic portion does not comprise the extracellular domain of the chimeric receptor (see, e.g., FIG. 21). In some embodiments, the binding of a ligand to the extracellular domain of the chimeric receptor activates the intracellular portion of the chimeric receptor (see, e.g., FIG. 22). In some embodiments, the delivery system will provide the ligand to bind the extracellular domain of the CAR to activate or polarize the target cell. Activation of the intracellular portion of the chimeric receptor in a macrophage or dendritic cell may polarize the macrophage or dendritic cell into an M1 or M2 form (see, e.g., FIGS. 23 and 24(A) and 25).

In some embodiments the delivery system includes a liposome. In certain embodiments the delivery system includes a lipid nanoparticle. In certain embodiments the delivery system includes a vesicle. In some embodiments the delivery system includes virus like particles (VLPs) and/or polymer-based particles. In some embodiments the delivery system includes nanoparticles. In some embodiments the nanoparticles are targeted nanoparticles target the nanoparticles directly to macrophages and/or monocytes.

In some embodiments the delivery system includes an activating agent. In particular embodiments the activating agent is a ligand for the extracellular domain and can be either protein based or lipid based. In some embodiments the activating agent is a lipopolysaccharide (LPS).

In some embodiments the delivery system also includes a nucleic acid inhibitor that decreases the expression of a protein involved in the pathway response for the degradation of the CAR. In some embodiments the nucleic acid inhibitor may be an RNA.

In certain embodiments, the extracellular domain may comprise an antibody, or a fragment there of, that binds to a ligand.

Embodiments include methods of polarizing a macrophage by contacting a macrophage comprising a chimeric receptor with a ligand for the extracellular domain of the chimeric receptor; binding the ligand to the extracellular domain of the chimeric receptor. The binding of the ligand to the extracellular domain of the chimeric receptor activates the cyptoplasmic portion and the activation of the cytoplasmic portion polarizes the macrophage.

Further embodiments include cells comprising a chimeric antigen receptor or nucleic acids encoding a chimeric antigen receptor.

In certain embodiments, the disclosure is a method for transfecting cell with a delivery system. In embodiments the cell is a leukocyte, and in some embodiments a monocyte, and in particular embodiments a macrophage or dendritic cell. In particular embodiments, the macrophage and/or monocytes in associated with a tumor. In a more particular embodiments, the macrophage is a tumor associated macrophage (TAM). In embodiments, the transduction of the cell with the delivery system will cause the cell to migrate to and/or become associated with a tumor.

In some embodiments, the method will introduce a nucleic acid encoding a chimeric antigen receptor into the cell. In some embodiments, binding of a ligand to the extracellular domain of the chimeric antigen receptor will activate the intracellular portion of the chimeric antigen receptor. In some embodiments, activation of the intracellular portion of the chimeric antigen receptor will polarize a transduced macrophage or dendritic cell into the M1 or M2 form. In further embodiments, the delivery system will also provide the ligand to the extracellular portion of the chimeric antigen receptor so the delivery system can both transform a naïve leukocyte or bind to the extracellular portion of a chimeric antigen receptor on a previously transformed leukocyte.

In embodiments, the delivery system includes a targeting agent. The targeting agent preferentially binds to the cell to which the delivery system is being directed. In certain embodiments, the targeting agent is protein, small molecule, glycoprotein, or an antibody or fragment thereof. In particular embodiments, the targeting agent is also an activating agent. In embodiments, the targeting agent preferentially binds to a leukocyte a monocyte, macrophage, or other dendritic cell. In some embodiments the targeting moiety includes a binding agent that is a ligand for receptors on particular cell types. In some embodiments the targeting agent is mannose.

These and other aspects of the disclosure will become apparent to the skilled artisan in view of the teachings contained herein.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fec.

FIG. 1(A) depicts a block diagram of the order of elements in the chimeric receptor TK1-MOTO1. FIG. 1(B) depicts the sequence of TK1-MOTO1 (SEQ ID NO:35). Amino acids 1-18 are a signal peptide (SP), amino acids 19-275 are an anti-TK1 ScFv, amino acids 276-290 are a GS linker, amino acids 291-313 are a TLR4 transmembrane domain, and amino acids 314-496 are a TLR4 cytosolic domain.

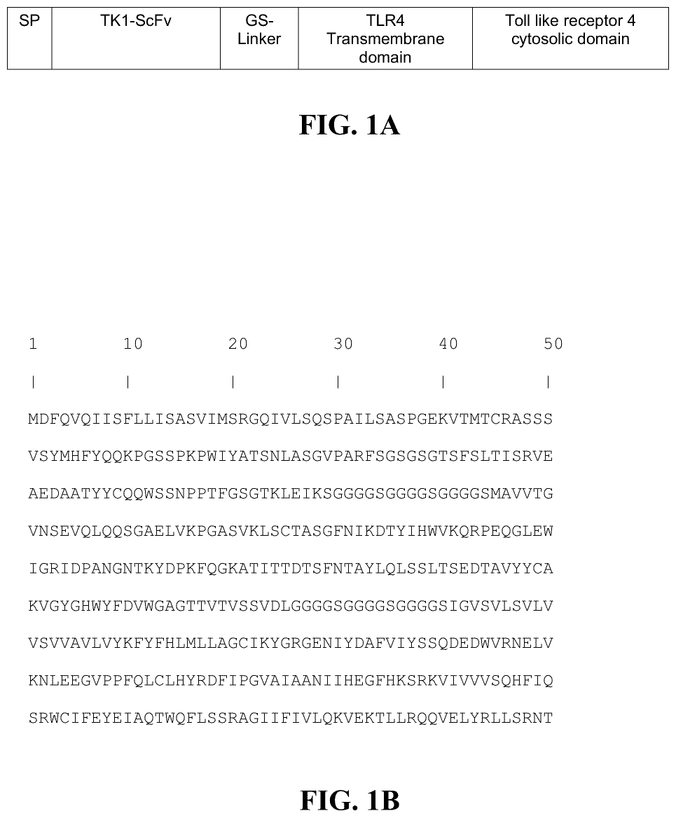

FIG. 2(A) depicts a block diagram of the order of elements in the chimeric receptor TK1-MOTO2. FIG. 2(B) depicts the sequence of TK1-MOTO2 (SEQ ID NO:36). Amino acids 1-18 are a signal peptide (SP), amino acids 19-275 are an anti-TK1 ScFv, amino acids 276-290 are a GS linker, amino acids 291-295 are a LRR short hinge, amino acids 296-318 are a TLR4 transmembrane domain, and amino acids 319-500 are a TLR4 cytosolic domain.

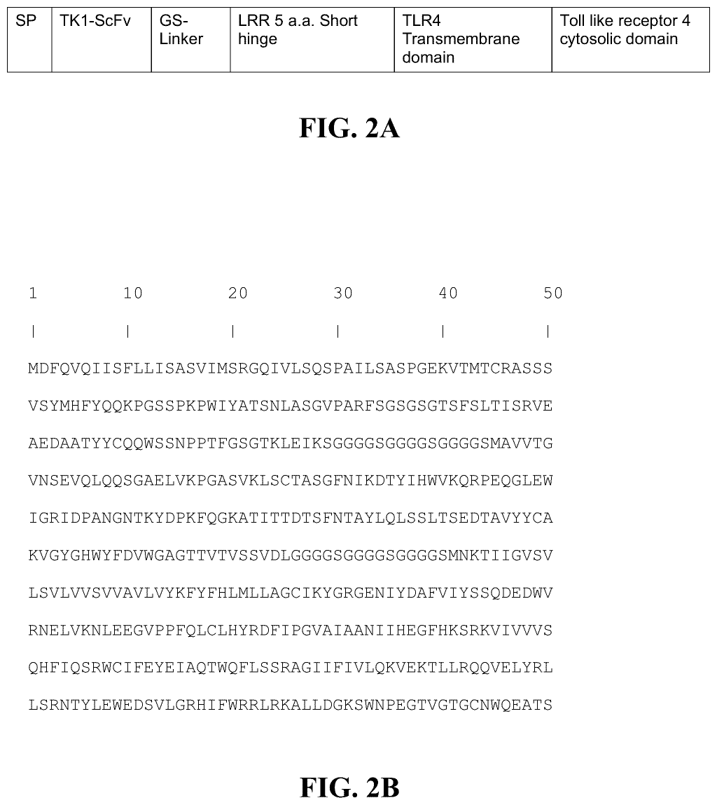

FIG. 3(A) depicts a block diagram of the order of elements in the chimeric receptor TK1-MOTO3. FIG. 3(B) depicts the sequence of TK1-MOTO3 (SEQ ID NO:37). Amino acids 1-18 are a signal peptide (SP), amino acids 19-275 are an anti-TK1 ScFv, amino acids 276-290 are a GS linker, amino acids 291-345 are a LRR long hinge, amino acids 346-368 are a TLR4 transmembrane domain, and amino acids 269-501 are a TLR4 cytosolic domain.

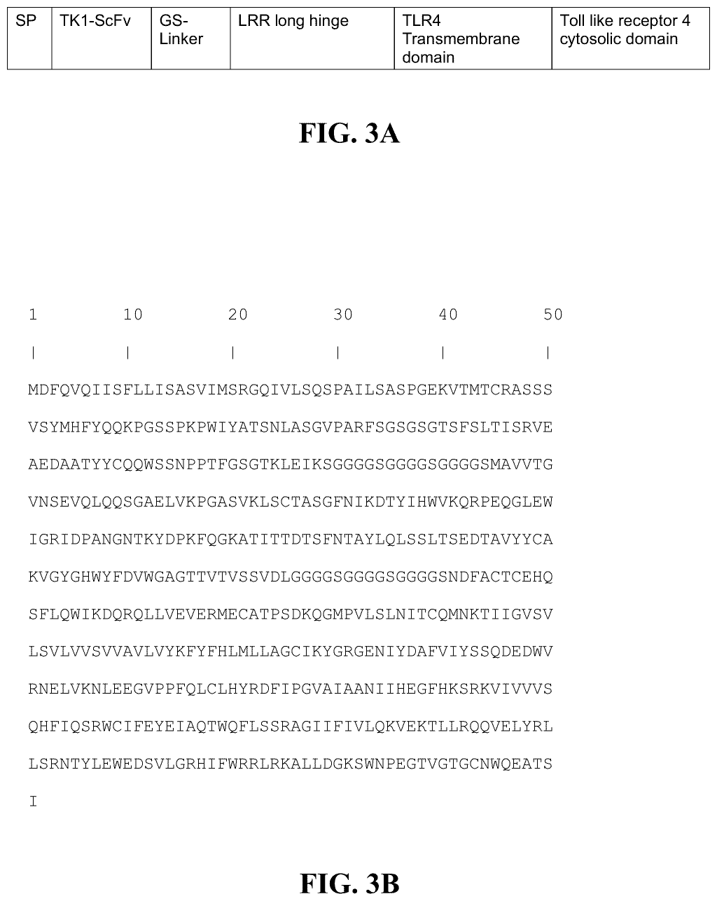

FIG. 4(A) depicts a block diagram of the order of elements in the chimeric receptor TK1-MOTO4. FIG. 4(B) depicts the sequence of TK1-MOTO4 (SEQ ID NO:38). Amino acids 1-18 are a signal peptide (SP), amino acids 19-275 are an anti-TK1 ScFv, amino acids 276-290 are a GS linker, amino acids 291-302 are an IgG4 short hinge, amino acids 303-325 are a TLR4 transmembrane domain, and amino acids 326-508 are a TLR4 cytosolic domain.

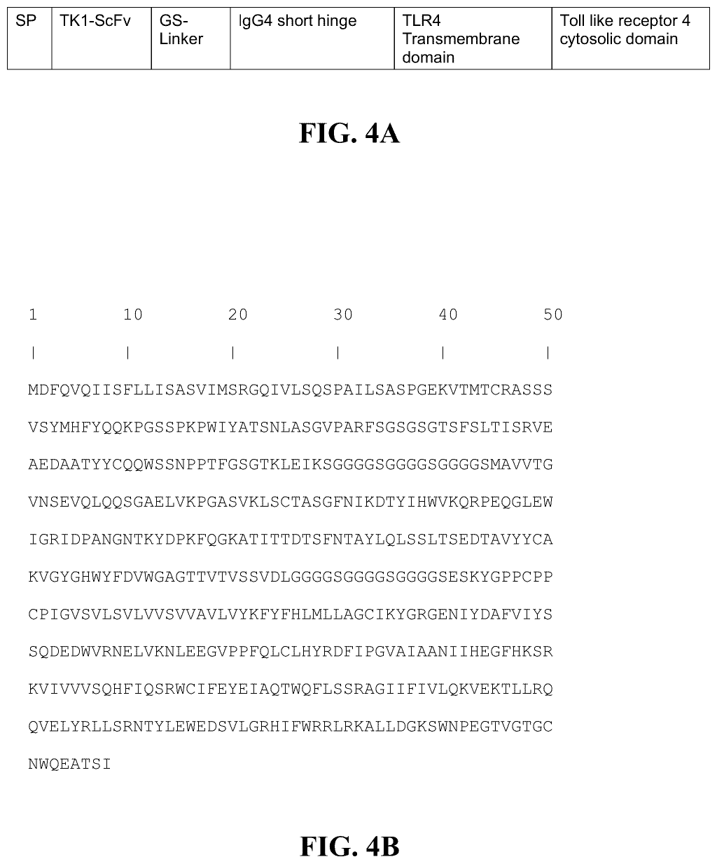

FIG. 5(A) depicts a block diagram of the order of elements in the chimeric receptor TK1-MOTO5. FIG. 5(B) depicts the sequence of TK1-MOTO5 (SEQ ID NO:39). Amino acids 1-18 are a signal peptide (SP), amino acids 19-275 are an anti-TK1 ScFv, amino acids 276-290 are a GS linker, amino acids 291-409 are an IgG 119 amino acid medium hinge, amino acids 410-432 are a TLR4 transmembrane domain, and amino acids 433-615 are a TLR4 cytosolic domain.

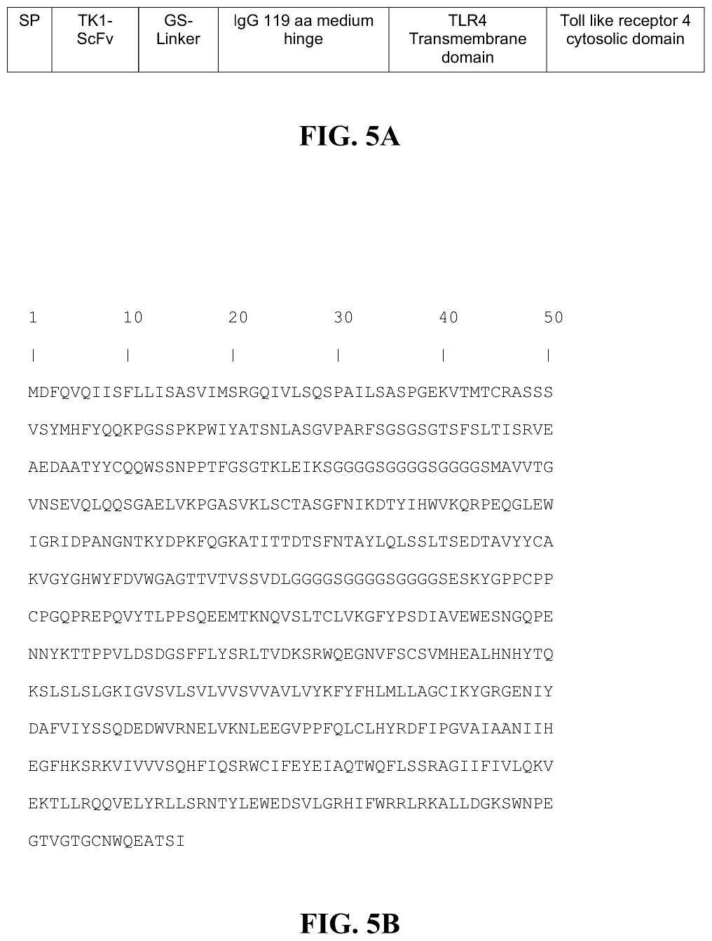

FIG. 6(A) depicts a block diagram of the order of elements in the chimeric receptor TK1-MOTO6. FIG. 6(B) depicts the sequence of TK1-MOTO6 (SEQ ID NO:40). Amino acids 1-18 are a signal peptide (SP), amino acids 19-275 are an anti-TK1 ScFv, amino acids 276-290 are a GS linker, amino acids 291-518 are an IgG4 long hinge, amino acids 519-541 are a TLR4 transmembrane domain, and amino acids 542-724 are a TLR4 cytosolic domain.

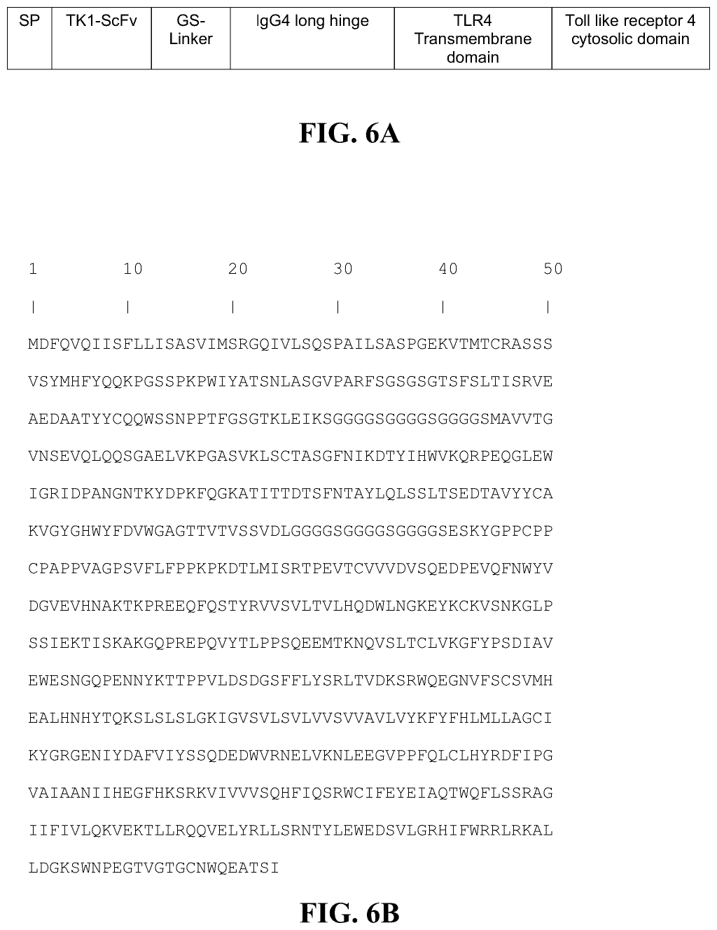

FIG. 7(A) depicts a block diagram of the order of elements in the chimeric receptor TK1-MOTO7. FIG. 7(B) depicts the sequence of TK1-MOTO7 (SEQ ID NO:41). Amino acids 1-18 are a signal peptide (SP), amino acids 19-275 are an anti-TK1 ScFv, amino acids 276-290 are a GS linker, amino acids 291-358 are mutated CD8 hinge with C339S and C356S, amino acids 359-381 are a TLR4 transmembrane domain, and amino acids 382-564 are a TLR4 cytosolic domain.

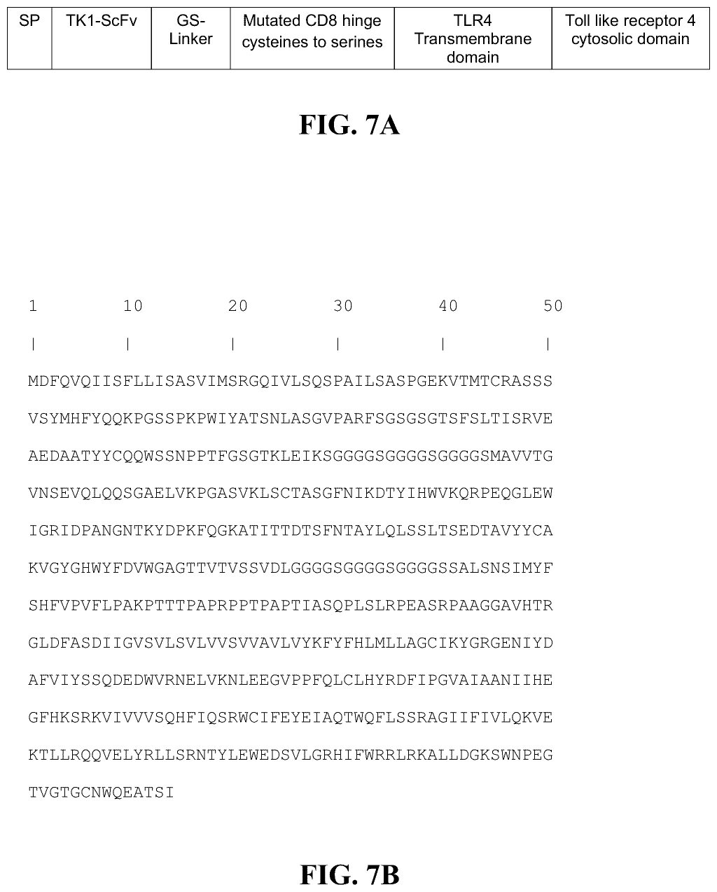

FIG. 8(A) depicts a block diagram of the order of elements in the chimeric receptor TK1-MOTO8. FIG. 8(B) depicts the sequence of TK1-MOTO8 (SEQ ID NO:42). Amino acids 1-18 are a signal peptide (SP), amino acids 19-275 are an anti-TK1 ScFv, amino acids 276-290 are a GS linker, amino acids 291-358 are a portion of a CD8 hinge, amino acids 359-381 are a TLR4 transmembrane domain, and amino acids 382-564 are a TLR4 cytosolic domain.

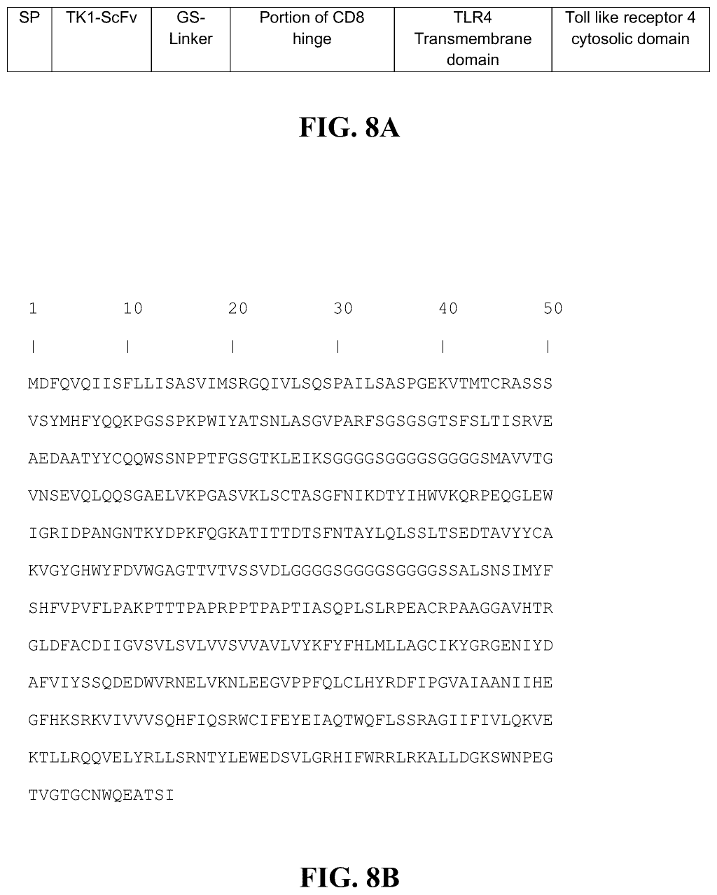

FIG. 9(A) depicts a block diagram of the order of elements in the chimeric receptor TK1-MO-FCGRA-CAR-1. FIG. 9(B) depicts the sequence of TK1-MO-FCGRA-CAR-1 (SEQ ID NO: 43). Amino acids 1-18 are a signal peptide (SP), amino acids 19-275 are an anti-TK1 ScFv, amino acids 276-290 are a GS linker, amino acids 291-311 are a FCGR3A transmembrane domain, amino acids 312-336 are a FCGR3A cytosolic domain, and amino acids 337-378 are a FCER1G cytosolic domain.

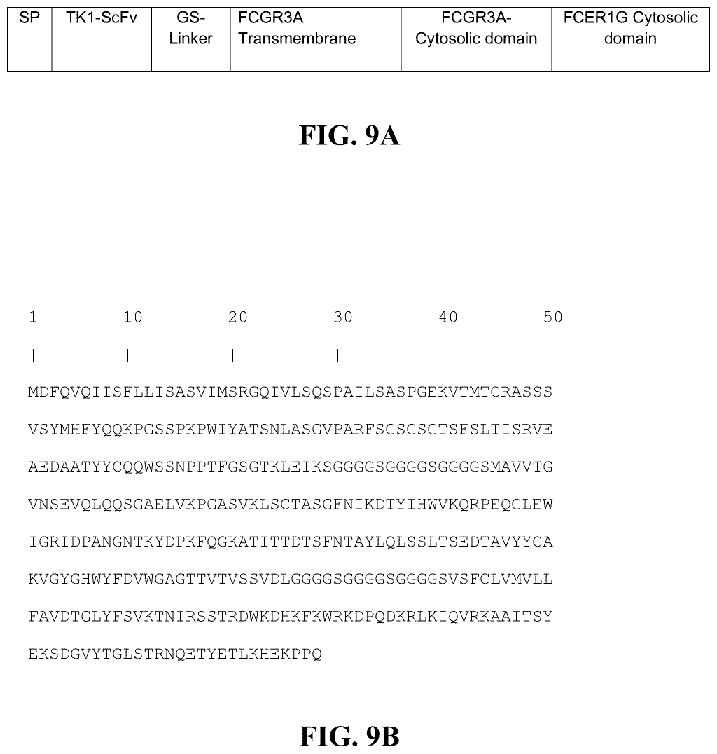

FIG. 10(A) depicts a block diagram of the order of elements in the chimeric receptor TK1-MO-FCGRA-CAR-2. FIG. 10(B) depicts the sequence of TK1-MO-FCGRA-CAR-2 (SEQ ID NO: 44). Amino acids 1-18 are a signal peptide (SP), amino acids 19-275 are an anti-TK1 ScFv, amino acids 276-290 are a GS linker, amino acids 291-358 are mutated CD8 hinge with C339S and C356S, amino acids 359-379 are a FCGR3A transmembrane domain, amino acids 380-404 are a FCGR3A cytosolic domain, and amino acids 405-446 are a FCER1G cytosolic domain.

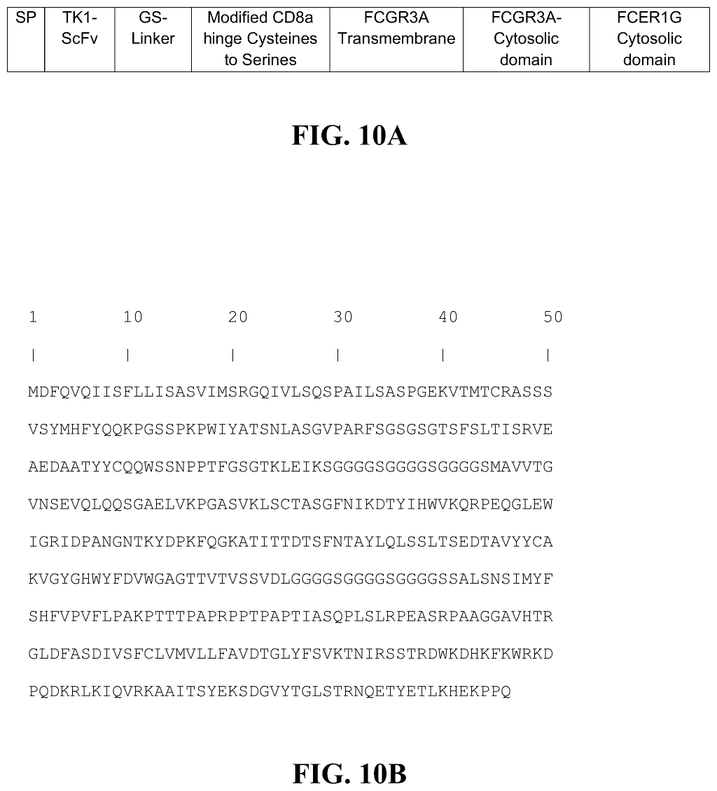

FIG. 11(A) depicts a block diagram of the order of elements in the chimeric receptor TK1-MO-FCGRA-CAR-3. FIG. 11(B) depicts the sequence of TK1-MO-FCGRA-CAR-3 (SEQ ID NO: 45). Amino acids 1-18 are a signal peptide (SP), amino acids 19-275 are an anti-TK1 ScFv, amino acids 276-290 are a GS linker, amino acids 291-358 are a portion of a CD8 hinge, amino acids 359-379 are a FCGR3A transmembrane domain, amino acids 380-404 are a FCGR3A cytosolic domain, and amino acids 405-446 are a FCER1G cytosolic domain.

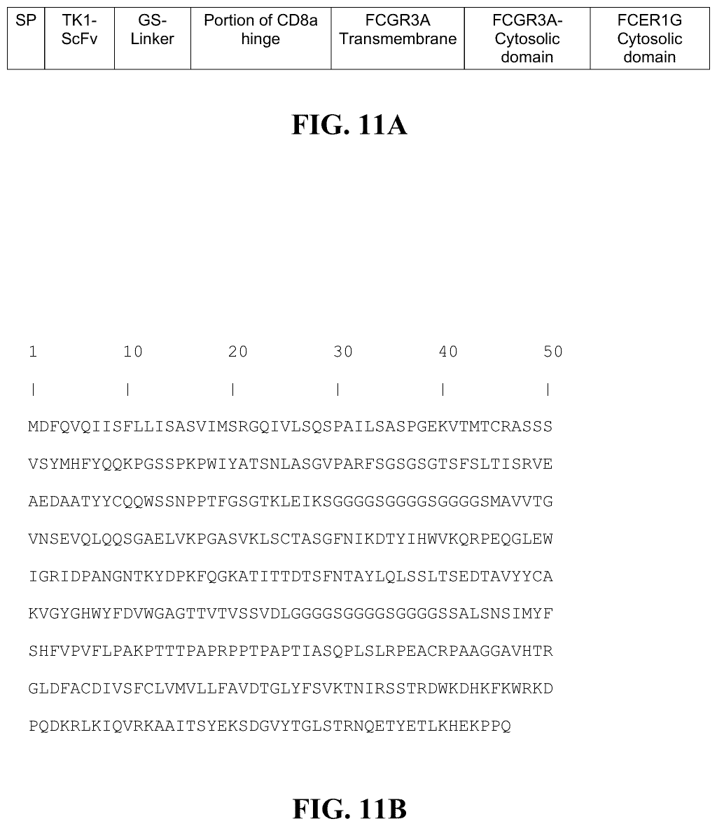

FIG. 12(A) depicts a block diagram of the order of elements in the chimeric receptor TK1-MO-FCGRA-CAR-4. FIG. 12(B) depicts the sequence of TK1-MO-FCGRA-CAR-4 (SEQ ID NO: 46). Amino acids 1-18 are a signal peptide (SP), amino acids 19-275 are an anti-TK1 ScFv, amino acids 276-290 are a GS linker, amino acids 291-303 are a IgG4 short hinge, amino acids 304-324 are a FCGR3A transmembrane domain, amino acids 325-349 are a FCGR3A cytosolic domain, and amino acids 350-391 are a FCER1G cytosolic domain.

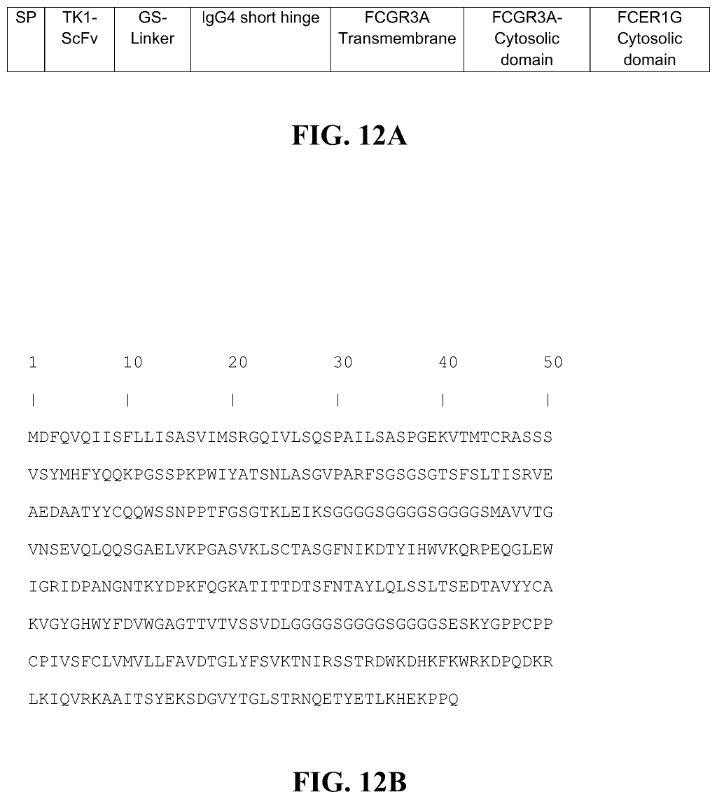

FIG. 13(A) depicts a block diagram of the order of elements in the chimeric receptor TK1-MO-FCGRA-CAR-5. FIG. 13(B) depicts the sequence of TK1-MO-FCGRA-CAR-5 (SEQ ID NO: 47). Amino acids 1-18 are a signal peptide (SP), amino acids 19-275 are an anti-TK1 ScFv, amino acids 276-290 are a GS linker, amino acids 291-409 are a IgG4 119 amino acid hinge, amino acids 410-430 are a FCGR3A transmembrane domain, amino acids 431-455 are a FCGR3A cytosolic domain, and amino acids 456-497 are a FCER1G cytosolic domain.

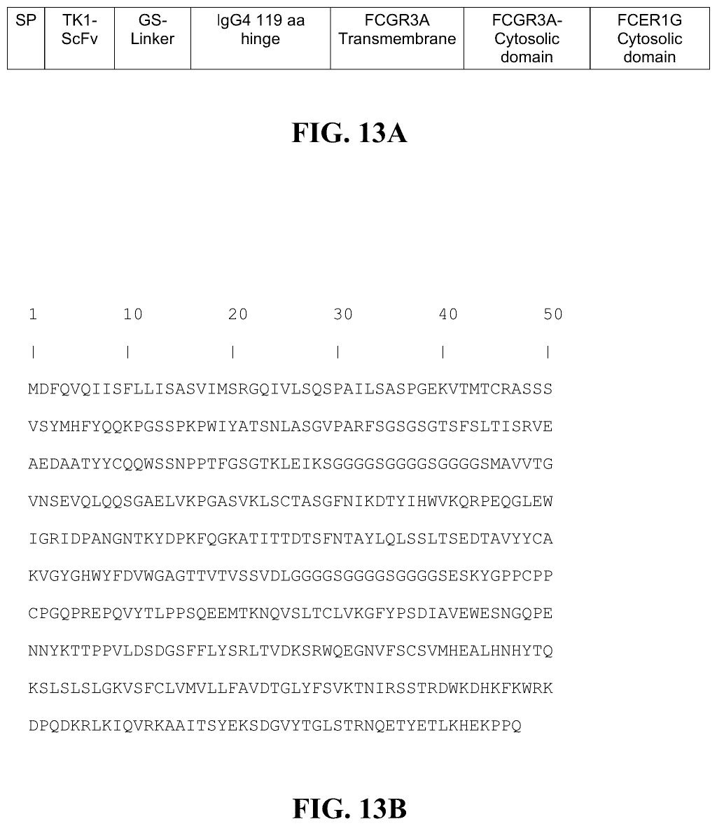

FIG. 14(A) depicts a block diagram of the order of elements in the chimeric receptor TK1-MO-FCGRA-CAR-6. FIG. 14(B) depicts the sequence of TK1-MO-FCGRA-CAR-6 (SEQ ID NO: 48). Amino acids 1-18 are a signal peptide (SP), amino acids 19-275 are an anti-TK1 ScFv, amino acids 276-290 are a GS linker, amino acids 291-519 are a IgG4 long hinge, amino acids 520-540 are a FCGR3A transmembrane domain, amino acids 541-565 are a FCGR3A cytosolic domain, and amino acids 566-607 are a FCERIG cytosolic domain.

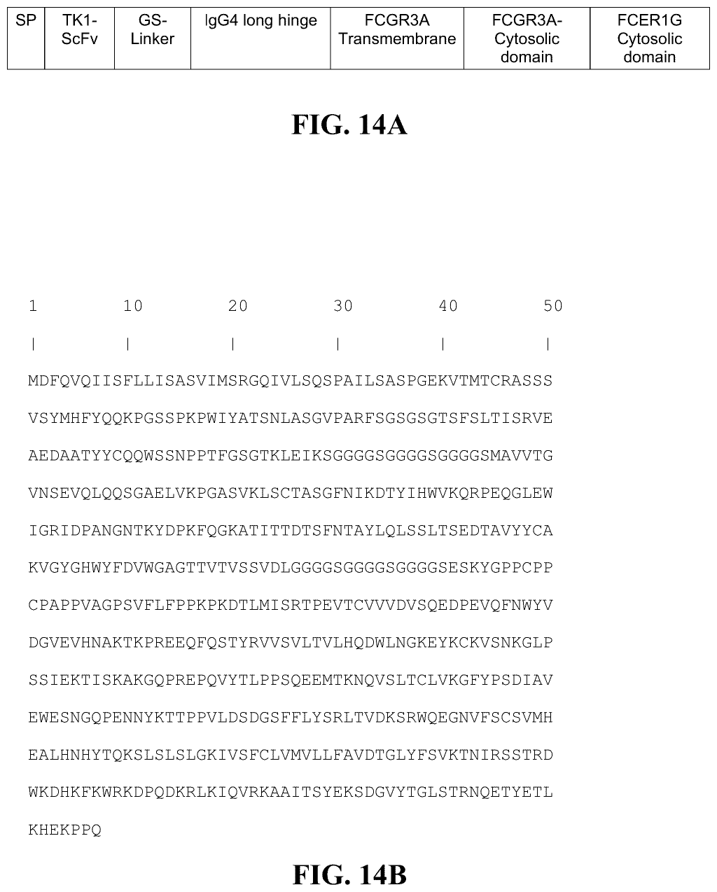

FIG. 15(A) depicts a block diagram of the order of elements in the chimeric receptor TK1-MO-FCG2A-CAR-1. FIG. 15(B) depicts the sequence of TK1-MO-FCG2A-CAR-1 (SEQ ID NO: 49). Amino acids 1-18 are a signal peptide (SP), amino acids 19-275 are an anti-TK1 ScFv, amino acids 276-290 are a GS linker, amino acids 291-312 are a FCGR2A transmembrane domain, amino acids 313-390 are a FCGR2A cytosolic domain.

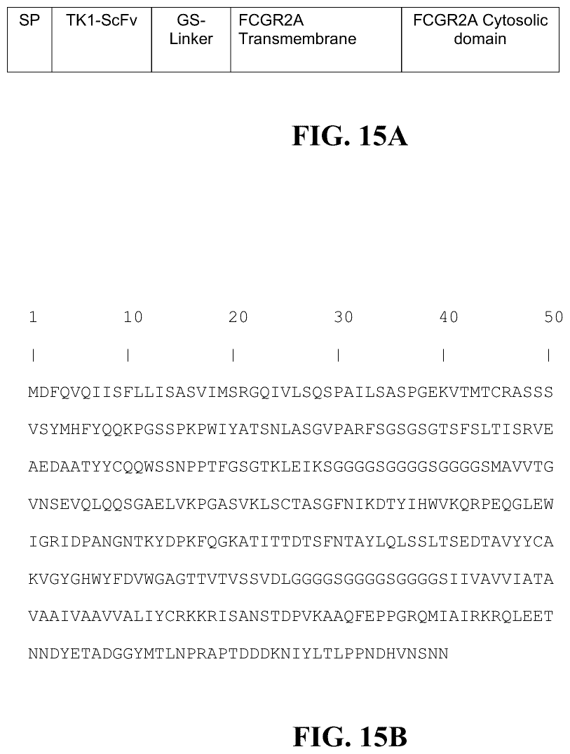

FIG. 16(A) depicts a block diagram of the order of elements in the chimeric receptor TK1-MO-FCG2A-CAR-2. FIG. 16(B) depicts the sequence of TK1-MO-FCG2A-CAR-2 (SEQ ID NO: 50). Amino acids 1-18 are a signal peptide (SP), amino acids 19-275 are an anti-TK1 ScFv, amino acids 276-290 are a GS linker, amino acids 291-358 are mutated CD8 hinge with C339S and C356S, amino acids 359-380 are a FCGR2A transmembrane domain, amino acids 381-458 are a FCGR2A cytosolic domain.

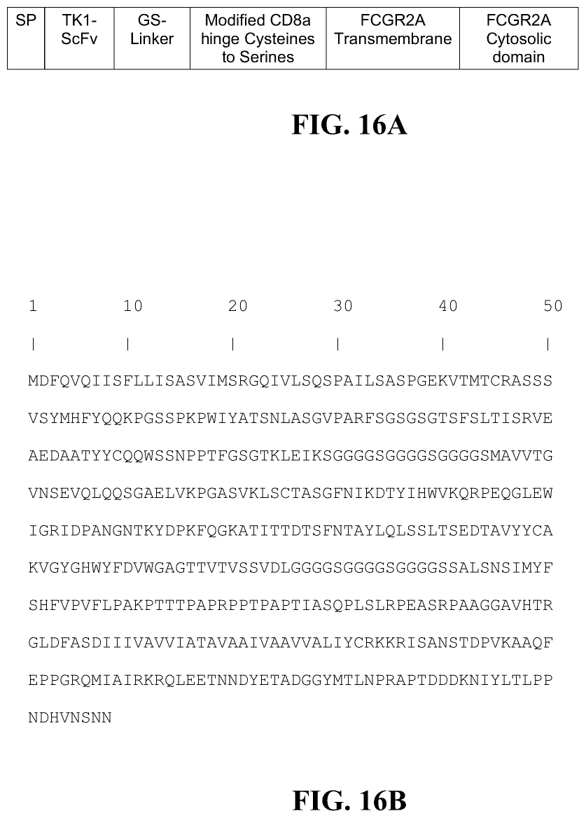

FIG. 17(A) depicts a block diagram of the order of elements in the chimeric receptor TK1-MO-FCG2A-CAR-3. FIG. 17(B) depicts the sequence of TK1-MO-FCG2A-CAR-3 (SEQ ID NO: 51). Amino acids 1-18 are a signal peptide (SP), amino acids 19-275 are an anti-TK1 ScFv, amino acids 276-290 are a GS linker, amino acids 291-358 are a portion of a CD8 hinge, amino acids 359-380 are a FCGR2A transmembrane domain, amino acids 381-458 are a FCGR2A cytosolic domain.

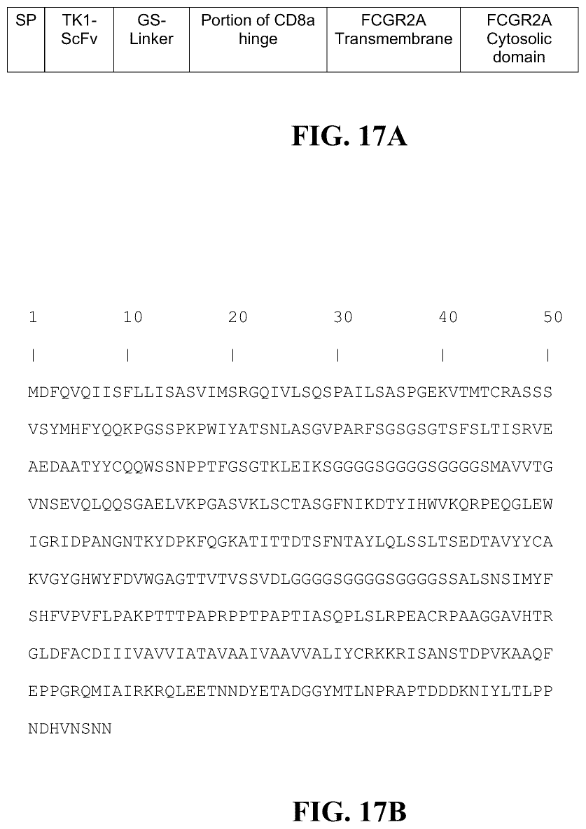

FIG. 18(A) depicts a block diagram of the order of elements in the chimeric receptor TK1-MO-FCG2A-CAR-4. FIG. 18(B) depicts the sequence of TK1-MO-FCG2A-CAR-4 (SEQ ID NO: 52). Amino acids 1-18 are a signal peptide (SP), amino acids 19-275 are an anti-TK1 ScFv, amino acids 276-290 are a GS linker, amino acids 291-303 are a IgG4 short hinge, amino acids 304-325 are a FCGR2A transmembrane domain, amino acids 326-403 are a FCGR2A cytosolic domain.

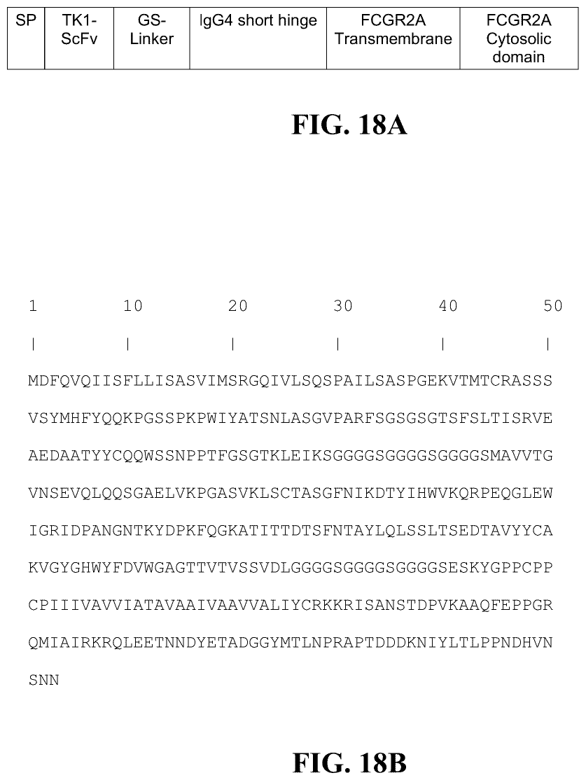

FIG. 19(A) depicts a block diagram of the order of elements in the chimeric receptor TK1-MO-FCG2A-CAR-5. FIG. 19(B) depicts the sequence of TK1-MO-FCG2A-CAR-5 (SEQ ID NO: 53). Amino acids 1-18 are a signal peptide (SP), amino acids 19-275 are an anti-TK1 ScFv, amino acids 276-290 are a GS linker, amino acids 291-409 are a IgG4 119 amino acid hinge, amino acids 410-431 are a FCGR2A transmembrane domain, amino acids 432-509 are a FCGR2A cytosolic domain.

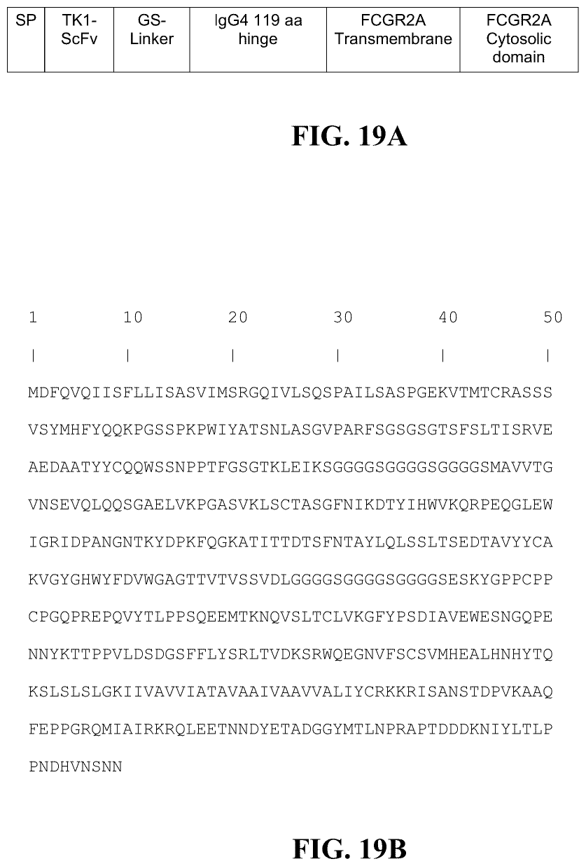

FIG. 20(A) depicts a block diagram of the order of elements in the chimeric receptor TK1-MO-FCG2A-CAR-6. FIG. 20(B) depicts the sequence of TK1-MO-FCG2A-CAR-6 (SEQ ID NO: 54). Amino acids 1-18 are a signal peptide (SP), amino acids 19-275 are an anti-TK1 ScFv, amino acids 276-290 are a GS linker, amino acids 291-519 are a IgG4 long hinge, amino acids 520-541 are a FCGR2A transmembrane domain, amino acids 542-619 are a FCGR2A cytosolic domain.

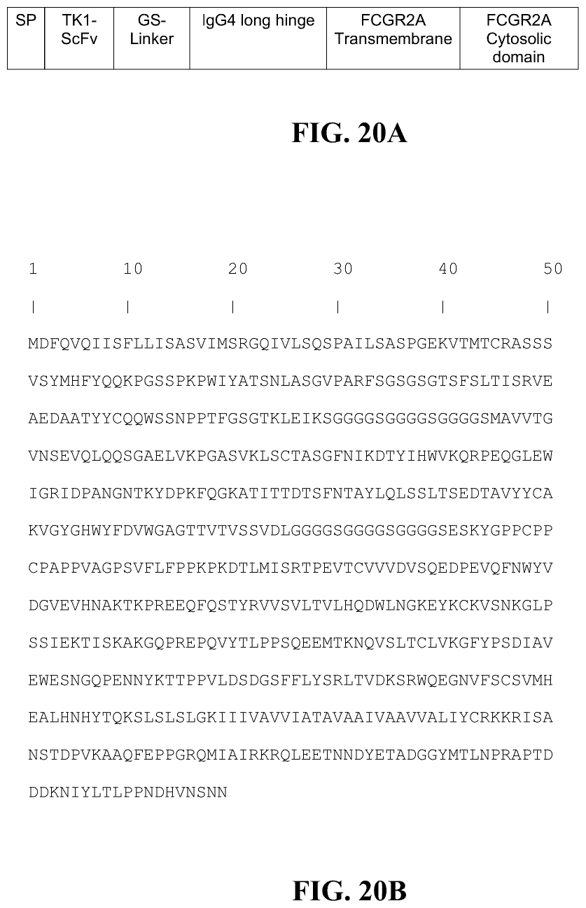

FIG. 21 is a schematic illustrating a chimeric receptor.

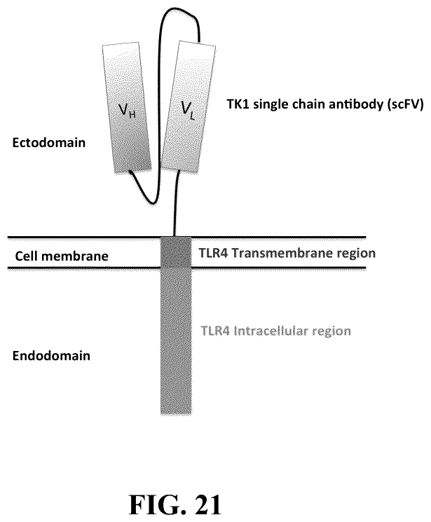

FIG. 22 is a schematic showing a macrophage expressing a chimeric receptor. As depicted, the chimeric receptor comprises the cytosolic domain of a toll like receptors, a transmembrane domain, and a ScFv specific for a ligand. The arrows depict signaling to polarize the macrophage upon the ScFv binding the ligand.

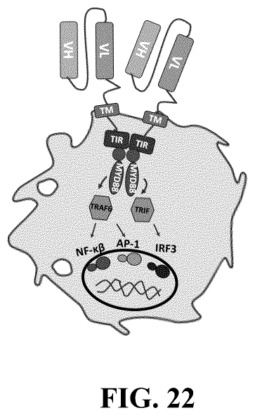

FIG. 23 is a schematic showing different macrophage receptors that could be utilized to build a chimeric receptor.

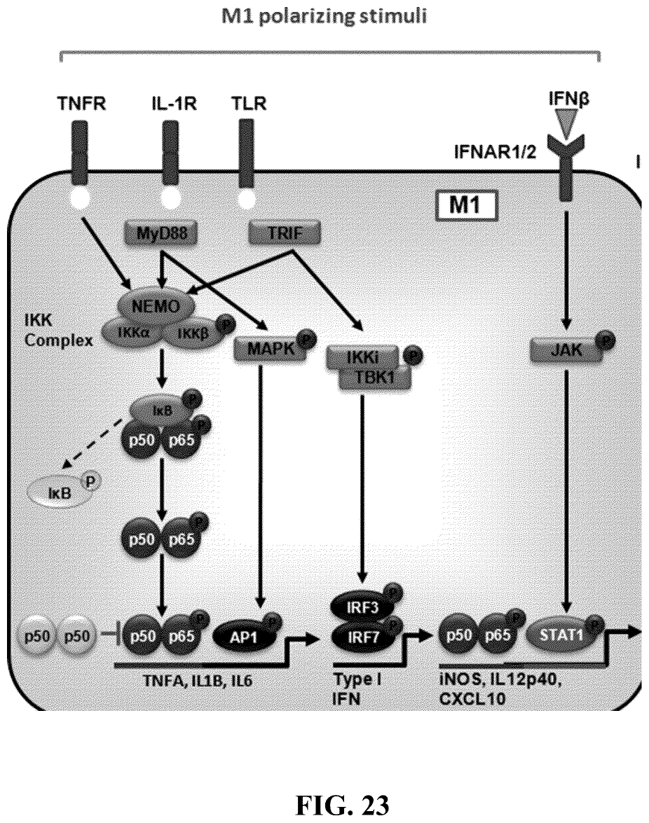

FIGS. 24(A) through 24(C). FIG. 24(A) is a schematic showing the Fc Gamma Receptor III signaling cascade leading to cell activation. FIG. 24(B) is a schematic showing the Fc Gamma Receptor III signaling cascade leading to inhibition of calcium flux and proliferation. FIG. 24(C) is a schematic showing the Fc Gamma Receptor III signaling cascade leading to apoptosis.

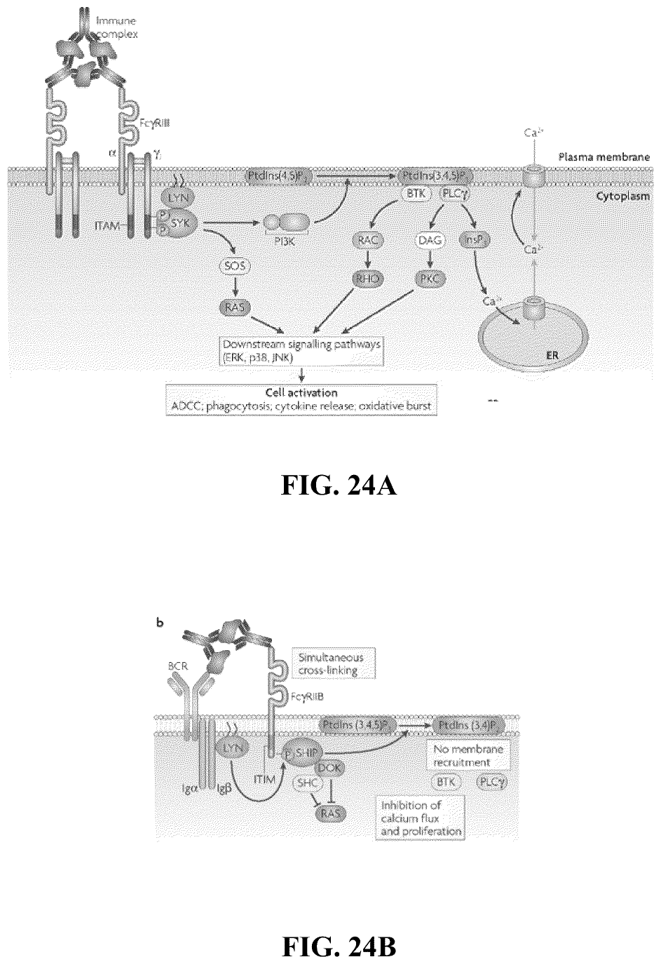

FIG. 25 is a schematic illustrating the Toll Like Receptor Signaling cascade.

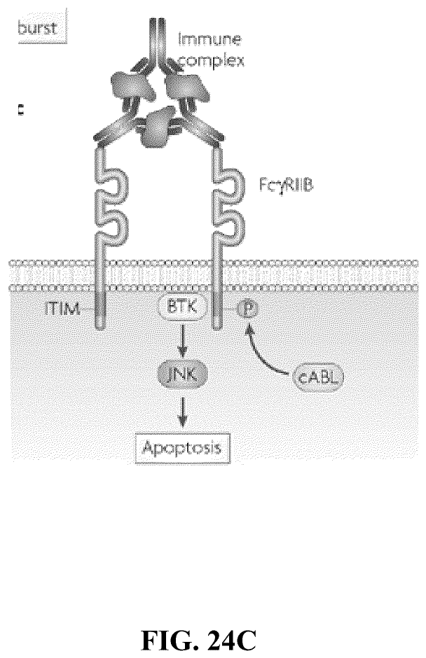

FIG. 26 presents graphs illustrating flow cytometry confirming that an expressed antibody fragment binds the ligand of interest.

FIG. 27 presents two images showing a phenotype change in macrophages after transduction with a chimeric receptor.

FIG. 28 presents two images confirming the expression from a vector encoding a chimeric receptor in monocytes.

FIG. 29 presents three scatter plots of fluorescence activated cell sorting demonstrating the expression of dTomato. The left most plot shows a control wherein only 0.58% of cells show fluorescence which would indicate expression of dTomato. The right two plots show a transduction efficiency of 27.1 percent after transduction.

FIG. 30 presents six scatter plots of fluorescence activated cell sorting demonstrating the retention of dye (Alexa 647), and the expression of CD80, CD163, CD206, and CD14 in macrophages transduced with a chimeric receptor.

FIG. 31 presents a histogram demonstrating the relative expression levels of CD80, CD163, CD206, and CD14 in macrophages transduced with a chimeric receptor.

FIG. 32 presents six images of transduced macrophages expressing a chimeric receptor interacting with a lung cancer cell line (NCI-H460).

FIG. 33 is a schematic showing an embodiment of the delivery system of the present disclosure, with a delivery vector delivering a nucleic acid to a cell.

FIG. 34 is a schematic showing an embodiment of the delivery system of the present disclosure, with an example of a nucleic acid inhibitor that decreases the expression of a protein that forms part of the pathway that targets the CAR for degradation and/or degrades the CAR in a mammalian cell, such a TLR4 inhibitor.

FIGS. 35A-35D present graphs and a histogram demonstrating the transfection of macrophages in vitro with nanoparticles. FIG. 35A. shows the viability of the cells following transfection with nanoparticles under a variety of transfection parameters. Viability decreases upon nanoparticle transfection due to the resulting activation of the cells upon uptake. FIG. 35B depicts MFI showing the relative intensity between the different treatments. FIG. 35C shows the total transfection efficiency rate (%) in each condition of treatment. FIG. 35D depicts a H histogram overlay of various transfections when compared to untreated control.

FIGS. 36A-B depicts in vivo data using a luciferase expressing vector to evaluate the transfection with nanoparticles a targeting tumor in mice. The data in FIGS. 36A-B, show levels of luciferase activity at 24 hr and 48 hr incubation respectively with the top two images being delivery via the tail vein injection and the bottom two images of each incubation period showing delivery via intraperitoneal injection.

FIG. 37 depicts the sequence of a RAGE construct (SEQ ID NO:55). Nucleic acids 1-57 encode a signal peptide (AZU1), nucleic acids 58-780 encode an SS ScFv, nucleic acids 781-1476 encode an IgG4 hinge, nucleic acids 1477-1665 encode the RAGE transmembrane and intracellular signaling domains.

FIGS. 38A-38F present graphs, qualitative visualization, and fluorescent binding images evaluating nanoparticle transfection of RAGE MOTO-CARs in primary human monocytes for both surface expression and target mesothelin protein binding. FIG. 38A shows a graph of the percent dTomato+ nanoparticle transfected in MOTO-CAR and mock cells. FIG. 38B shows two graphs with levels of mesothelin binding, one graph depicting the mesothelin binding in transfected cells with percent dTomato+ GFP+ and the other graph showing the amount of mesothelin binding with mean fluorescent intensity (MFI) for both mock and RAGE MOTO-CAR cells. FIG. 38C presents scatter plots demonstrating the qualitative fluorescent binding within each evaluated construct compared to untransfected controls. FIG. 38D are images taken using confocal microscopy visualizing the qualitative fluorescence in cells labeled with a nuclear stain (DAPI), dTomato, and GFP for both mock and RAGE MOTO-CAR cells. FIGS. 38E and 38F demonstrate that RAGE CAR-T cells preferentially killed target mesothelin positive HCC-1806 cells.

FIGS. 39A and 39B. FIG. 39A presents scatter plots demonstrating the nanoparticle transfected RAGE MOTO-CARs phagocytosis events of mesothelin expressing cells, HCC-1806 and MDA-MB-231 Msln in comparison to mock controls. FIG. 39B is a graph of the significant increase in overall percent (%) phagocytosis between the RAGE MOTO-CAR and the mock (dKIT) controls.

FIG. 40 depicts the targeted killing of mesothelin positive HCC-1806 cells with nanoparticle transfected RAGE CAR-T cells in comparison to controls dKIT and target only assays, as measured by normalized cell index.

FIGS. 41A and 41B. FIG. 41A depicts the change in mesothelin positive HCC-1806 tumor volume over a time series in NSGS mice injected with transfected cells comprising DNA encoding a MOTO-CAR in comparison to the control injected with cells comprising DNA encoding a mock control (dKIT). FIG. 41B depicts the difference in tumor weight (g) between the control cells and those injected with MOTO-CAR engineered cells.

FIGS. 42A-42D depicts graphs, histograms, and images showing the nanoparticle transfection efficiency of MOTO-CARs given different starting nanoparticle parameters. FIG. 42A shows the transfection percentage (%) as well as the MFI for four different nanoparticle parameters in comparison to untreated cells, including Low DNA, Low DNA-Media, High DNA, and High DNA-Media. FIG. 42B shows the normalized viability percentage (%) and calculated cell count of nanoparticle transfected and untransfected cells for the four different nanoparoticle parameters in comparison to untreated cells including Low DNA, Low DNA-Media, High DNA, and High DNA-Media. FIG. 42C depicts a histogram of the nanoparticle transfected cell percentage for the four different parameters in comparison to untreated cells including Low DNA, Low DNA-Media, High DNA, and High DNA-Media, across three separate cell donors. FIG. 42D depicts an image of the nanoparticle transfected and untransfected cells for the four parameters in comparison to untreated cells.

FIGS. 43A-43C show quantitative graphs, histograms, and charts for the target cell binding and trogocytosis of nanoparticle transfected macrophages. FIG. 43A presents scatter plots of CD45+ effector cells and their relative transfection efficiency and target cell binding. FIG. 43B is a histogram that shows the relative double positivity between controls and MOTO-CAR assays. FIG. 43C are graphs that provide the double positive population percentage (%) in the controls and the MOTO-CAR assays, as well as the “fold increase” comparison between the Low DNA and High DNA-Media assays.

FIGS. 44A-44D show the reduction in tumor growth using nanoparticle transfection of various CAR constructs. FIG. 44A shows the normalized cell index for a time series for the CAR constructs CD3ζ. MS-TLR4, Hu-TLR4, Hu-RAGE IFNγ, CD32-CD28, and a control. FIG. 44B shows the individual normalized cell index levels for control versus the construct Hu-RAGE IFNγ, CD35. Hu-TLR4, CD3ζ-CD28. FIG. 44C shows the combined bar graph of normalized cell index at time points 7, 11, 13, 15, and 18 days for each of the CAR constructs: CD3ζ, MS-TLR4, Hu-TLR4, Hu-RAGE IFNγ, CD3ζ-CD28, and a control. FIG. 44D shows the individual bar graphs for day 11, 13, 15, and 18 for each of the CAR constructs and the control.

FIG. 45 depicts the sequence of a RAGE-IFNγ construct (SEQ ID NO:55). Nucleic acids 990-1046 encode a signal peptide (AZU1), nucleic acids 1047-1763 encode an SS1 scFv, nucleic acids 1770-2459 encode an IgG4 hinge, nucleic acids 2466-2657 encode the RAGE transmembrane and intracellular signaling domain, nucleic acids 2658-2714 encode a P2A site, and nucleic acids 2715-3215 encode IFNγ.

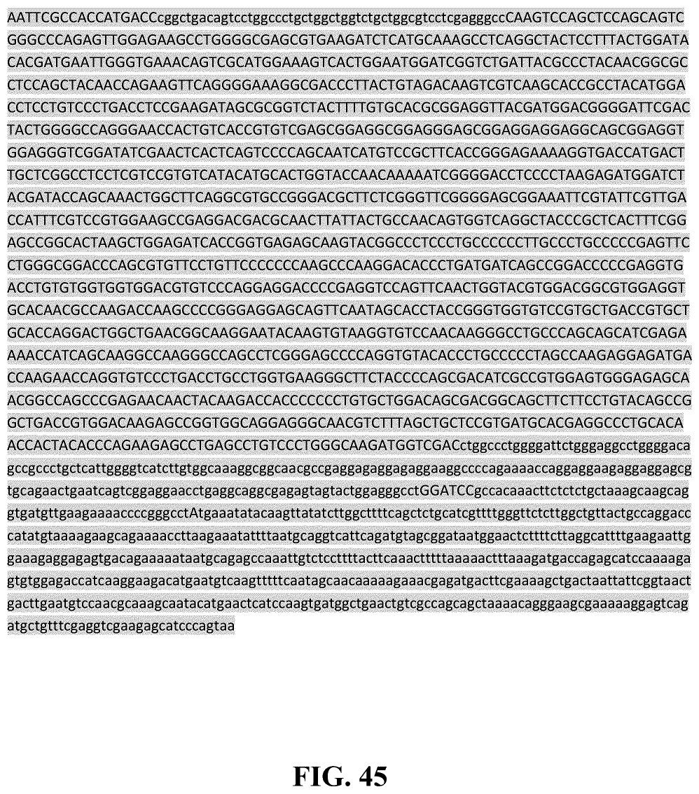

### DETAILED DESCRIPTION

Described herein is a delivery system and a method of using a delivery system to transduce a cell with a nucleic acid. In certain embodiments, the delivery system will preferentially a cell type of interest. In particular embodiments, the cell type of interest may be a leukocyte, and in some embodiments a monocyte and in particular embodiments a macrophage or dendritic cell. Examples of nucleic acids include those encoding a protein of interest, such as a CAR. In some embodiments the delivery system will also provide a ligand for the CAR. In some embodiments the ligand for the CAR is an activating agent that activates the cell, in some embodiments the cell is a leukocyte, in particular embodiments the cell is a monocyte or a dendritic cell, where the activating agent polarizes the macrophage into either M1 or M2 macrophages. In further embodiments the delivery system and method of using the delivery system will include a nucleic acid inhibitor that will decrease the expression of a protein that forms part of the pathway that degrades the CAR in a mammalian cell. In further embodiments, described herein are the cells, for example the leukocytes that have been transduced with nucleic acid encoding a CAR and a nucleic acid inhibitor that decreases the expression of proteins that forms part of the pathway that degrades the CAR in a mammalian cell.

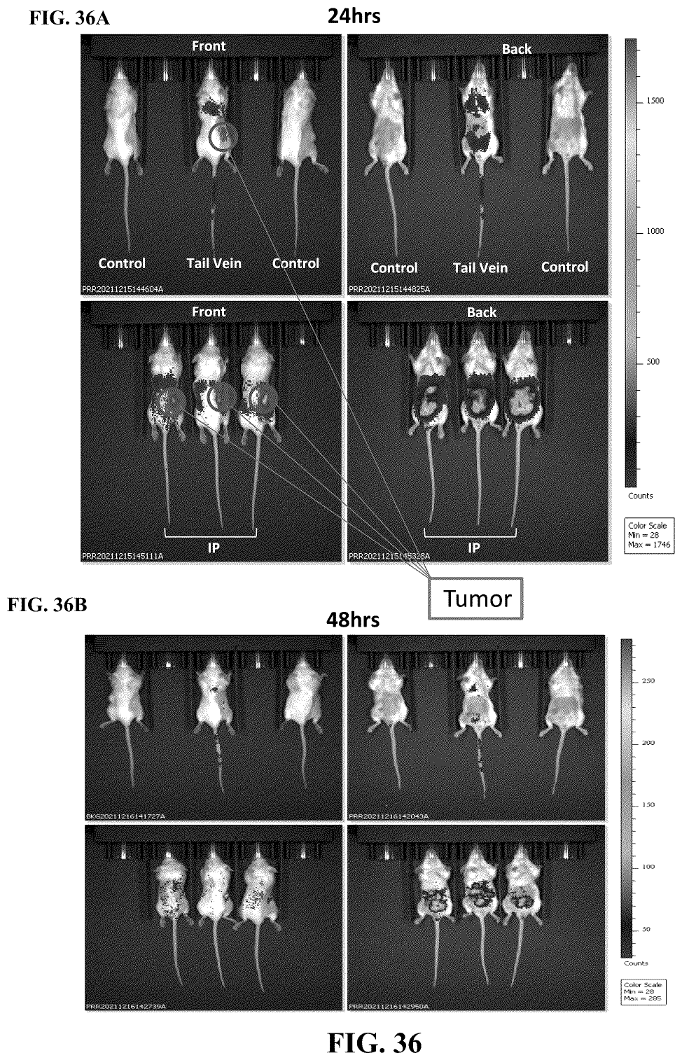

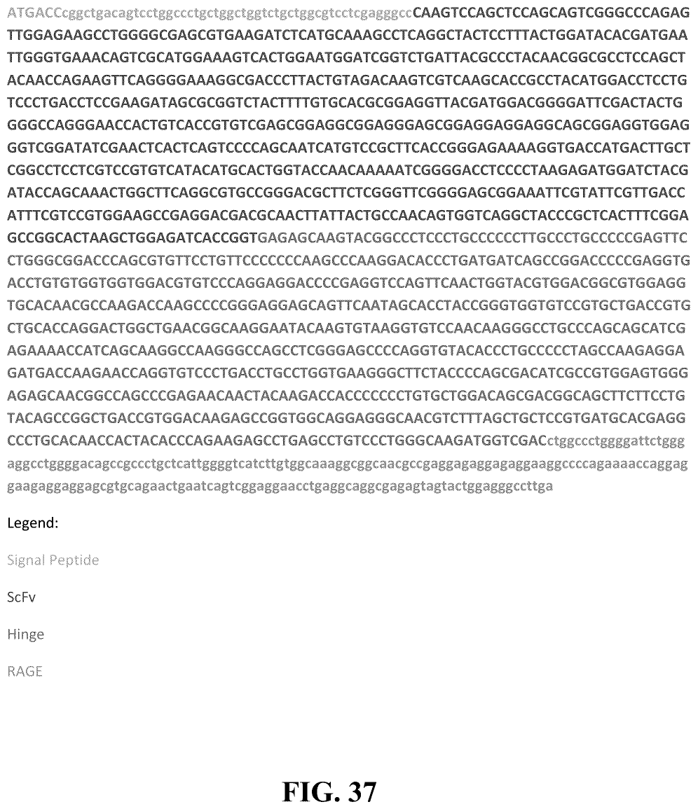

The delivery system of the present disclosure, will include, as a non-limiting example, a delivery vector, such as a cationic liposome (or LNP or Micelle), which will be carrying nucleic acid. In some embodiments the nucleic acid will be a chimeric antigen receptor mRNA, or a DNA with a nuclear transport protein as depicted in FIG. 33. In some embodiments the nucleic acid will also include a nucleic acid inhibitor that decreases the expression of proteins that form a part of the pathway that degrades the CAR in a mammalian cell, such as the TCR shRNA depicted in FIG. 33. The delivery vector will be targeted for specific cells, such as leukocytes, or particularly macrophages, or dendritic cells with a targeting agent on the surface of the delivery vector. In some embodiments this targeting agent will be a ligand for receptors on a macrophage or a dendritic cell, and in some embodiments may be a T cell ligand as depicted in FIG. 33. In some embodiments the targeting agent, such as a ligand, will bind to a receptor on the target cell, such as a receptor on a macrophage which will trigger endocytosis of the delivery vector into the cell. In some embodiments the vacuole formed by the endocytosis of the delivery vector will be processed releasing the nucleic acid the delivery vector was carrying. In some embodiments the nucleic acid and the nucleic acid inhibitor as depicted in FIG. 34 will be released into the cell. As a non-limiting example, the nucleic acid is RNA which will then be translated in the cell to produce a protein, which will be a chimeric antigen receptor (CAR). The CAR will then be processed in the cell and eventually expressed on the cell surface as depicted in FIG. 33. In some embodiments the nucleic acid inhibitor as depicted in FIG. 34 will inhibit, for example, TCR translation. In some embodiments, the CAR will also function as a receptor for a ligand on the delivery vector. In some embodiments when the ligand on the delivery vector binds to the CAR it will activate the cell. In some embodiments the cell activation will be to polarize a macrophage or dendritic cell into either an M1 or and M2 macrophage.

In some embodiments the delivery system, including nanoparticles as described in this disclosure target cells of the tumor microenvironment, and in particular the TAMs of the tumor microenvironment. The tumor microenvironment can promote tumor growth and formation by stimulating cell proliferation and angiogenesis. One key mediator of the tumor microenvironment are tumor associated macrophages (TAMs). Without being limited by this theory, it is believed that the TAM-mediated paracrine signaling where macrophage derived factors promote angiogenesis and activate the neoplastic cells and promote stemlike features in the cells, exacerbating tumor progression, metastasis, and even chemoresistance. As discussed in this disclosure, monocyte infiltration into a tumor is mediated by chemokines (e.g., CCL2, CCL5, and CXCL12), CSF-1, and components of the complement cascade. Once monocytes are in the tumor, the tumor environment rapidly promotes their differentiation into tumor-conditioned macrophages. TAMs also promote angiogenesis which increases tumor growth.

Without being limited by theory, it is believed that TAMs are biased away from an M1 type macrophage, expressing M2 protumor markers. M2-like TAMs have been identified by the hemoglobin-scavenger receptor CD163, the macrophage scavenger receptor 1 CD204, the mannose receptor CD206, CD68, and the T-cell immunoglobulin and mucin-domain containing protein 3 (Tim-3). In some embodiments the nanoparticle delivery system targets the tumor microenvironment and specifically TAMs. The CARs disclosed herein, when expressed and activated in TAMs, the transduced cells will no longer serve a tumor supporting role. Such converted TAMs may also recruit an immune response against the neoplastic cells.

Without being bound by any particular theory, it is believed that the transduced macrophages will phagocytose or trogocytose neoplastic cells. Transduced macrophages may also secrete signals which help polarize untransduced macrophages to participate in an anti-neoplastic cell response. Any taken up proteins from the neoplastic cells may then be presented to the immune system by the transduced macrophages and used to mount an immune response targeting the neoplastic cells.

### Delivery Vector

The present disclosure provides for a delivery vector in the delivery system that can deliver a nucleic acid to a target cell. Foreign nucleic acids, (e.g., DNA, RNA, and artificial nucleic acids) potently stimulates the innate immune response, particularly type 1 interferon (IFN) production. This occurs through a pathway dependent upon DNA sensor cyclic guanosine monophosphate-adenosine monophosphate synthase (cGAS) and the downstream adaptor protein stimulation of IFN genes (STING). The present disclosure includes the use of a delivery vector that can deliver nucleic acid to the cytosol of a target cell within a subject without inducing an immune response.

Methods of introducing a nucleic acid into a host cell are known in the art, and any known method can be used to introduce a nucleic acid into a cell, and by way of non-limiting example a leukocyte. Suitable methods include, e.g. viral or bacteriophage infection, transfection, transduction, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like, including but not limiting to exosome delivery.

Polynucleotides may be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18:1127-1133 (2011) (which focuses on non-viral delivery vehicles for siRNA that are also useful for delivery of other polynucleotides).

Suitable systems and techniques for delivering a nucleic acid of the disclosure include lipid nanoparticles (LNPs). As used herein, the term “lipid nanoparticles” includes liposomes irrespective of their lamellarity, shape or structure and lipoplexes as described for the introduction of nucleic acids and/or polypeptides into cells. These lipid nanoparticles can be complexed with biologically active compounds (e.g., nucleic acids and/or polypeptides) and are useful as in vivo delivery vehicles. In general, any method known in the art can be applied to prepare the lipid nanoparticles comprising one or more nucleic acids of the present disclosure and to prepare complexes of biologically active compounds and said lipid nanoparticles. Examples of such methods are widely disclosed, e.g. in Biochim Biophys Acta 1979, 557:9; Biochim et Biophys Acta 1980, 601:559; Liposomes: A practical approach (Oxford University Press, 1990); Pharmaceutica Acta Elelvetiac 1995, 70:95; Current Science 1995, 68:715; Pakistan Journal of Pharmaceutical Sciences 1996, 19:65; Methods in Enzymology 2009, 464:343). Particularly suitable systems and techniques for preparing LNP formulations comprising one or more nucleic acids and/or polypeptides of the present disclosure include, but are not limited to, those developed by Intellia (see e.g., WO2017173054A1), Alnylam (see, e.g., WO2014008334A1), Modernatx (see., e.g., WO2017070622A1 and WO2017099823A1), TranslateBio, Acuitas (see, e.g., WO2018081480A1), Genevant Sciences, Arbutus Biopharma, Tekmira, Arcturus, Merck (see, e.g., WO2015130584A2), Novartis (see, e.g., WO2015095340A1), and Dicerna; all of which are herein incorporated by reference in their entireties.

In some embodiments the LNP or other delivery vector may also include an adjuvant. In another aspect, the present disclosure relates, in part, to a method of delivering a nucleic acid molecule to a subject. In one embodiment, the method comprises administering a therapeutically effectively amount of one or more LNPs or compositions of the present invention to the subject. In some embodiments, the LNP or the composition delivers the nucleic acid molecule to a target cell. In some embodiments the target cell is a leukocyte. In some embodiments the target cell is a monocyte. In some embodiments the target cell is a macrophage or a dendritic cell.

In some embodiments, the LNP or the delivery vector is administered by an intradermal delivery route, subcutaneous delivery route, intramuscular delivery route, intraventricular delivery route, intrathecal delivery route, oral delivery route, intravenous delivery route, intratracheal delivery route, intraperitoneal delivery route, in utero delivery route, or any combination thereof.

In one embodiment, the method comprises a single administration of the LNP or the delivery vector. In some embodiments, the method comprises multiple administrations of the LNP or the delivery vector. In further embodiments the delivery vector can be an non-viral technique including by not limited to lipid based lipoplexes, polymer based polyplexes, peptide-based polyplexes, such as cell-penetrating peptides (CPP) and nuclear localization signals, poly-L-lysine (PLL), cationic liposomes, “biological particles”, namely peptide transduction domains, virus like particles, gesicles, and exosomes (see, e.g., Ni et al., Synthetic approaches for nucleic acid delivery: choosing the right carriers, Life (Basel), 2019 September; 9 (3): 59, incorporated by this reference).

In some embodiments the delivery system includes virus like particles. An association of viral proteins is referred to as a “virus like particle” (VLP) which comprises a covalently coupled or otherwise linked association of at least two viral coat proteins. In some embodiments the VLP comprises all of the different viral coat proteins. The VLP does not contain any replicating nucleic acid and is by itself thus not capable of causing an infection. In some embodiments the VLP can self-assemble. In some embodiments the VLP is a combination of structural capsid proteins from different viruses. In some embodiments, proteins, nucleic acids, or small molecules can be attached to the VLP surface to target the VLP to particular cell types, by way of a non-limiting example targeting macrophages or dendritic cells.

In some embodiments the delivery vector is a lipoplex or a genosome. A lipoplex is a lipid and DNA complex used to deliver nucleic acid. Anionic and neutral lipids can be used to construct a lipoplex. In some embodiments cationic lipids can be used to construct a lipoplex. Lipoplexes formed with cationic lipids can interact with the cell membrane and facilitate endocytosis of the lipoplex into the cell.

In some embodiments the delivery vector is a polymersome, a polyplex, or a dendrimer.

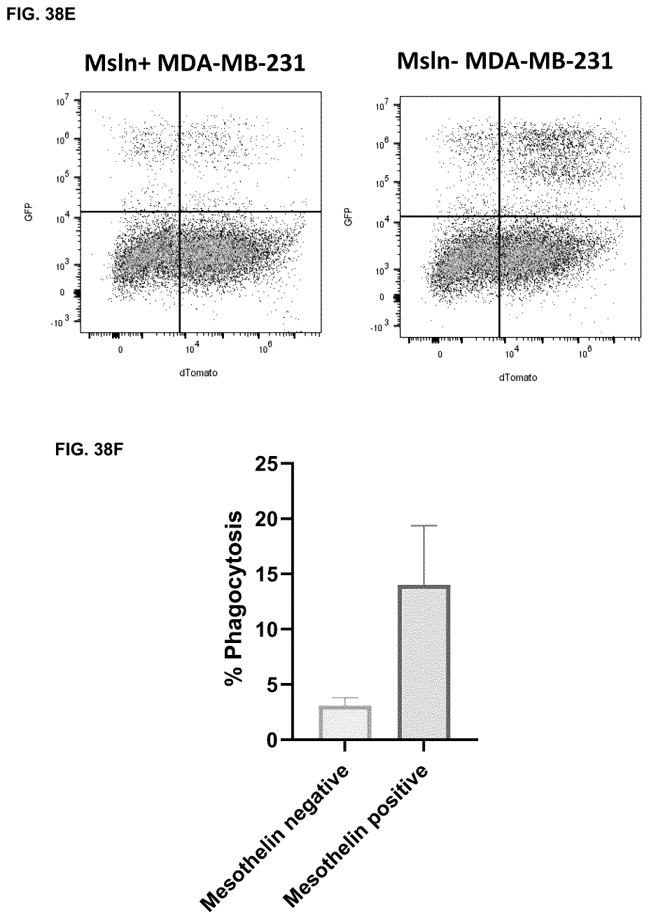

In some embodiments, the delivery vector is able to deliver nucleic acid to a target in an immunocompetent animal. As is depicted in FIGS. 36A-36B, the delivery system is capable of transfecting macrophages in mice with competent immune systems was unexpected, and is an important step forward in successfully transducing target cells, particularly macrophages, and even more particularly TAMs. This shows that the delivery system can deliver a construct to macrophages in a mouse that are localized to tumors with a competent immune system. In the case of transfecting macrophages, this means that the transfected macrophage will either have been directed to and target the tumor cells or that the delivery system delivers the construct to macrophages already at the site of the tumor, such as TAMs. Such transduced macrophages can also utilize the entire intact immune system to target and attack tumor cells. In showing this transfection in vivo, the mice in FIGS. 36A-36B, were either given a control, were given intraperitoneal (“IP”) injection with the delivery device, or were given an intravenous (“IV”) tail vein injection with the delivery device.

### Targeting Agent

Host mechanisms for particle processing are, at some level, highly evolved and difficult to by-pass, despite the best efforts of materials engineering. In the present disclosure, this provides an advantage to target and delivery nucleic acid to monocytes. The highly evolved mononuclear phagocytic system (MPS) is a function of particle opsonization upon contact with blood and rapid recognition of these opsonins via the MPS. Macrophages recognize opsonized proteins, specific surface chemistries, and other surface and biological characteristics that mark these nanoparticles, similar to analogous microparticle precedents, for clearance and/or toxicological fates.

Macrophages and other phagocytes are leukocytic cells capable of phagocytizing or taking up bacteria, cellular debris, and particles through energy-consuming membrane-engulfing as a characteristic phenotype. Their primary role is early response to foreign material contamination and its clearance. Macrophages have been known to uptake foreign materials within a matter of minutes, increasing their rates of phagocytosis for positively charged and bacteria-specific proteins.

Macrophages are key in vivo participants in normal inflammatory and immunological processes. As active phagocytes, they display a spectrum of phenotypes, spanning pro-inflammatory to prohealing, and appear capable of reversible transformations between different distinct functional forms. Certain macrophage forms are essential for the destruction and removal of hazardous materials, pathogens, and damaged or abnormal tissues; these native roles are also likely involved in nanoparticle processing. Macrophages also play an essential role in normal wound healing, prompting local angiogenesis and tissue neogenesis. Known also to play a primary role in the macroscale foreign body response to engineered biomaterial implants, macrophages initiate local fibrosis and unresolved chronic inflammation around implants that is not readily eliminated. Evidence suggests that local microenvironmental factors and cues drastically alter macrophage phenotype and differentiation states. This includes altered macrophage morphology, surface receptor expression and function, that ultimately affect material recognition and uptake patterns necessary for nanoparticle interactions and nanomaterials processing in vivo.

Mature macrophages are terminally differentiated forms of circulating hematopoietic premature precursor monocytes or derive from the tissue precursors in which they reside. Both blood-derived and tissue-resident macrophages participate in macrophage-nanoparticle interactions.

In vivo, the host particle surveillance and clearance systems (i.e., MPS or tissue-resident phagocytes) do not encounter bare nanomaterials. The immediate host biological conditioning produces protein adsorption to the biomaterial surface upon blood or tissue contact. The adsorbed protein coating, referred to as “corona” in the nanomaterials literature and also “opsonins” in the drug delivery literature, matures over time in vivo to an equilibrium state largely unknown for nanomaterials in blood. This time-dependent blood protein adsorption process, and what characteristics of nanoparticles initiate desirable and adverse effects, as well as how presence and conformation of adsorbed proteins influence the presentation of nanoparticles to phagocytes will be increasingly important to understand as a determinant of their clearance. Protein opsonization is rapid and has been well-known to “prime” particles for MPS recognition and clearance.

Changing particle surface energies (e.g., hydrophilicity/hydrophobicity) and imposing immobilized steric barriers (e.g., grafted polymer brush surfaces) may also decrease protein adsorption and subsequent phagocytic recognition of nanomaterials. For example, hydrophilic poly(ethylene glycol) (PEG) has often been immobilized in many forms and approaches to provide a brush-like steric barrier that is shown to reduce protein adsorption and is correlated with increased blood circulation times for some particles. Dextran layers employed on commercial iron oxide MRI agent nanoparticles (i.e., FERIDEX™) may serve the same role. Nanoparticle curvature, topography and surface energy represent only a few select physicochemical characteristics that can be altered to modify nanomaterials interfacial adsorption processes with proteins that affect their biological interactions (see, e.g., Gustafson et al., Nanoparticle Uptake: The Phagocyte Problem, Nano today, 2015 Aug. 10 (4): 487-510 incorporated herein by this reference).

Macrophages have evolved distinct pathogenic and foreign material recognition mechanisms. Many nanomaterial uptake and cellular processing mechanisms parallel normal immunological pathogenic processing, suggesting conservation in cellular recognition and pathway regulation. A variety of native surface receptors, called pattern-associated recognition receptors (PRRs), are able to recognize antigenic or epitope presentation patterns from pathogen surfaces or within damaged tissues. Pathogen surface patterns are conserved across a variety of microorganisms, termed pathogen-associated molecular patterns (PAMPs). PAMPs identify injury or cell death patterns, termed damage-associated molecular patterns (DAMPs) [104]. DAMPs usually correspond to host tissues undergoing necrosis and as host-elicited danger signals that initiate local recruitment of immune cells. Because foreign material, pathogens and damaged native tissues present patterns recognized by phagocyte surface receptors, nanoparticles could also potentially present analogous molecular patterns due to their protein adsorption, or associated specifically to the raw material physicochemical properties. Further, these patterns could potentially initiate normal inflammatory events mediated by phagocytic cells. Four specific macrophage surface receptors include: (1) toll-like receptors, (2) mannose receptors, (3) scavenger receptors, and (4) Fc receptors.

Phagocytosis, a primary mechanism for nanoparticle uptake by macrophages, is broadly used to describe actin rearrangement and pseudopodial envelopment of large bodies into cells. Phagocytosis is usually associated with Fc- and complement mediated (CR)-mediated receptors, enveloping material by cell membrane dynamics in a zipper-like fashion. Only certain classes of cells, usually termed “professional phagocytes.” have this type of cytoskeletal rearrangement capability. These include macrophages, neutrophils, dendritic cells, monocytes, and only in special cases, endothelial and secretory epithelial cells. Cellular pseudopodial vesiculations appear concurrent with internal granule movements and subsequent granule fusion within the cell. Usually following phagocytosis, vesicles containing the foreign material fuse with lysosomal compartments, which then undergo pH reductions capable of destroying pathogens. Phagocytosis is limited to particle sizes below 10 microns and more commonly below 6 microns. Rates of phagocytosis vary widely and change depending on cell type, activation state, culture conditions or particle biological conditioning (e.g., endotoxin or protein exposure).

Cell-targeted cargo delivery can be enhanced by methods to reliably select specific intracellular organelles to better predict and enable site-specific action. After foreign materials are taken up into endosomes or phagosomes, they fuse with lysosomal compartments used by cells to neutralize foreign material with isolated, focal heavy enzymatic digestion and reduced pH. This vesicle fusion allows cells abilities to degrade or remove hostile materials and pathogens from their intracellular environment for inactivation. However, these vesicles also encompass recognition motifs (i.e. TLRs, integrins, etc.) that can traffic ingested material to other specific cellular compartments. These motifs, in turn, can be utilized for site-directed delivery.

Generally, about 95% of every nanomaterial dosing to blood non-specifically targets the MPS filtration organs comprising fenestrated vasculature with high populations of committed phagocytes. This occurs independently of any surface-immobilized target motifs (e.g., peptides, ligands, antibodies, etc.). Non-specific targeting of nanomaterials (i.e., scavenging) is the host default processing pathway for any and all systemized nanomaterials. Toxicity associated with high loading of nanomaterials to MPS organs is represented by cell stress biomarkers, specifically subsets of cytokines, chemokines and reactive oxygen species (ROS). Following the production dynamics of specific cytokine and chemokine markers and possible mechanistic dissection of downstream effects of nanoparticle-induced stress in vivo can be used to follow toxicity responses.

In the present disclosure the targeting agent is a ligand specific for the targeted cells. In some embodiments the ligand is a monocyte ligand, for example ligands to the toll-like receptor, GM-CSF, CD14, CD16, CD64, CD115, CD192, CX2CR1, CD226, CD284, CD155, or any combination thereof. In some embodiments the targeting agent may be lipid based. Activation of the toll-like receptor 4 initiates the NF-κB pathway and inflammatory cytokine production as part of the innate immune system. see, e.g., FIG. 33.

Binding of ligands to the extracellular domains of TLRs causes a rearrangement of the receptor complex and triggers the recruitment of specific adaptor proteins to the intracellular domain, thus initiating a signaling cascade.

TLR4 signals through adaptor molecules such as MyD88, toll/IL-1 receptor domain-containing adaptor protein (TIRAP), toll/IL-1 receptor domain-containing adaptor inducing interferon-β (TRIF) and TRIF-related adaptor molecule (TRAM) to activate transcription factors such as nuclear factor (NF)-κB, activator protein 1 (AP-1), and interferon regulatory factors (IRFs). These transcription factors then initiate the transcription of a specific set of genes involved in proinflammatory, anti-viral, and anti-bacterial responses and genes that control cell survival and apoptosis. TLR4 signaling has been divided into MyD88-dependent (mediated by MyD88) and MyD88-independent (mediated by TRIF) pathways. These two pathways also mediate the intracellular signaling of other TLRs, enabling the interaction between TLR4 and other TLRs at different levels from adaptor molecules to transcription factors. MyD88 is an essential part of the signaling cascade of all TLRs except for TLR3. In contrast. TRIF only interacts with TLR3 and TLR4. The M1 transcription factors include STAT1, C/EBP-α, C/EBP-8, IRF9, KLF6, NF-kB, AP1, HIF1α. The M2 transcription factors include PPARγ, STAT3. STAT6, C/EBP-β, IRF4, KLF4, GATA3, c-MYC.

In the MyD88-dependent pathway, TLR4, through TIRAP, recruits MyD88 to activate IL-1R-associated kinase (IRAK)-4 and IRAK-1, which then associate with tumor necrosis receptor-associated factor (TRAF)-6 and transforming growth factor-β-activated kinase 1 (TAK-1). These activate the complex inhibitor of NF-kB kinase (IKK), formed by NEMO, IKKα e IKKβ, which phosphorylates and degrades IκBα (inhibitor of NF-κB), allowing nuclear translocation of NF-kB (normally sequestered in the cytoplasm by ligation to IκBα). NF-κB leads to expression of effectors genes (TNF-α, IL-6, and IL-12). The MyD88-dependent pathway can also activate p38 and c-Jun N-terminal kinase (JNK), leading to AP-1 activation followed by transcription of genes involved in regulation of cell proliferation, morphogenesis, apoptosis, and differentiation.

In the MyD88-independent pathway, TLR4, through TRAM, recruits TRIF. This recruits TRAF3 which associates with TRAF family member associated NF-kB activator (TANK), TBK1 (TANK binding kinase 1) and IKKi with subsequent phosphorylation and nuclear translocation of IRF-3. IRF-3 leads to IFN-β transcription. In MyD88-independent pathway. TRIF also associates with the receptor-interacting serine-threonine kinase (RIP)-1 to activate NF-κB. NF-kB induction in the MyD88-dependent pathway occurs with fast kinetics, whereas NF-kB activation in the MyD88-independent pathway occurs with slower kinetics (see, e.g., Soares et al. The role of lipopolysaccharide/toll-like receptor 4 signaling in chronic liver diseases, Hepatol. Int., 2010 December; 4 (4): 659-672 incorporated herein by this reference).

The targeting agent used in the present disclosure to direct the delivery vector to the target cell, by way of non-limiting example a monocyte, can also be used as an activator for the target cell. When the target cell contacts the delivery vector, the targeting agent would activate the target cell. In some embodiments the target cell has been previously transformed and the extracellular domain of the CAR can be bound by the targeting agent as a ligand to activate the target cell.

In some embodiments the delivery system includes additional targeting moieties on the delivery vector to deliver nucleic acid to a particular cell type. In some embodiments the additional targeting moiety includes a binding agent that is a ligand for receptors on particular cell types, for example monocytes, and by way of a non-limiting example on macrophages and dendritic cells. In some embodiments a carbohydrate moiety, such as a carbohydrate receptor, for example a C-type lectins can be used on the surface of the delivery vector. In one non-limiting example the C-type lectin receptor is the mannose receptor (CD206), which is highly expressed on macrophages, including pro-inflammatory M1 macrophages.

By way of a non-limiting example of a delivery vector, a phospholipid-based and PEGylated nanoparticle (NP) can be modified with targeting peptides, such as mannose, on its surface to target macrophages, such as tumor-associated macrophages (TAMs) in the tumor microenvironment. The NP can have a structure and function controlled by both a peptide that can target the scavenger receptor B type 1 (SR-B1) and a peptide that has a higher specificity to, for example, M2 polarized macrophages than to other leukocytes. These two peptides may be fused into one single entity and incorporated in phospholipid based, PEGylated NPs. Other examples can include mannosylated solid lipid nanoparticles, mannosylated thiolated chitosan and chitosan-polyethyleneimine NPs, mannosylated polyamidoamine (PAMAM) dendrimers, or N-acetyl-mannosylated gold NPs (see, e.g., Poupot et al., Multivalent nanosystems: targeting monocytes/macrophages, International Journal of Nanomedicine, 2018; 13:5511-5521 incorporated herein by this reference).

### Nucleic Acid Inhibitor

The delivery system may also include inhibitory nucleic acids, such as inhibitory RNA as depicted in FIG. 34. Wherein the delivery system comprises a nucleic acid encoding a protein, the inhibitory RNA may target proteins that would target for degradation and/or degrade the encoded protein. The delivery system can include nucleic acids such as RNA, including microRNA, shRNA, and siRNA that are designed to suppress, inhibit, disrupt or otherwise silence genes or products that play a role in degrading the encoded protein. In a non-limiting example of the present disclosure, a nucleic acid encoding the chimeric receptor may also include nucleic acid that inhibit the genes or expression of proteins that would target the chimeric receptor. In a non-limiting example, the nucleic acid encoding the chimeric antigen would include an shRNA at the 3′ end of the transcript that would reduce expression of, for example, a TLR4 degradation protein (e.g. RFN216 or RAB7b). The expression of an shRNA would not be integrated into the genome but is transiently transfected into the cell to prolong the life of the chimeric antigen receptor. Because the shRNA will not be integrated into the genome, once the construct is no longer present to create, for example, a double stranded RNA hairpin, the effects of the knockdown would no longer be exhibited in the successfully transfected cells, see FIG. 34.

### Chimeric Antigen Receptors

Chimeric antigen receptors (CARs) comprise a cytoplasmic domain, a transmembrane domain, and an extracellular domain. In embodiments, the cytoplasmic domain comprises a cyptoplasmic portion of a receptor that when activated polarizes a macrophage. In further embodiments, a wild-type protein comprising the cytoplasmic portion does not comprise the extracellular domain of the chimeric receptor. In embodiments, the binding of a ligand to the extracellular domain of the chimeric receptor activates the intracellular portion of the chimeric receptor. Activation of the intracellular portion of the chimeric receptor may polarize the macrophage into an M1 or M2 macrophage. In certain embodiments the delivery system will provide the ligand to the extracellular domain of the chimeric receptor.

In certain embodiments, the cytoplasmic portion of the chimeric receptor may comprise a cytoplasmic domain from a toll-like receptor, myeloid differentiation primary response protein (MYD88) (SEQ ID No: 19), toll-like receptor 3 (TLR3) (SEQ ID No:1), toll-like receptor 4 (TLR4) (SEQ ID No: 3), toll-like receptor 7 (TLR7) (SEQ ID No:7), toll-like receptor 8 (TLR8) (SEQ ID No:9), toll-like receptor 9 (TLR9) (SEQ ID No:11), myelin and lymphocyte protein (MAL) (SEQ ID No:21), interleukin-1 receptor associated kinase 1 (IRAK1) (SEQ ID No:23), low affinity immunoglobulin gamma Fc region receptor III-A (FCGR3A) (SEQ ID No:15), low affinity immunoglobulin gamma Fc region receptor II-a (FCGR2A) (SEQ ID No:13), high affinity immunoglobulin epsilon receptor subunit gamma (FCER1G) (SEQ ID No: 19), or sequences having at least 90% sequence identity to a cytoplasmic domain of any one of the foregoing. In certain embodiments, the cytoplasmic portion is not a cytoplasmic domain from a toll-like receptor. FCGR3A. IL-1 receptor, or IFN-gamma receptor. In embodiments, the cytosolic portion can be any polypeptide that, when activated, will result in the polarization of a macrophage.

In further embodiments, examples of ligands which bind to the extracellular domain may be, but are not limited to. Thymidine Kinase (TK1). Hypoxanthine-Guanine Phosphoribosyltransferase (HPRT), Receptor Tyrosine Kinase-Like Orphan Receptor 1 (ROR1). Mucin-16 (MUC-16). Epidermal Growth Factor Receptor vIII (EGFRvIII). Mesothelin. Human Epidermal Growth Factor Receptor 2 (HER2), Carcinoembryonic Antigen (CEA), B-Cell Maturation Antigen (BCMA), Glypican 3 (GPC3). Fibroblast Activation Protein (FAP). Erythropoietin-Producing Hepatocellular Carcinoma A2 (EphA2). Natural Killer Group 2D (NKG2D) ligands. Disialoganglioside 2 (GD2), CD19, CD20, CD30, CD33, CD123. CD133, CD138, and CD171. In certain embodiments, the ligand is not TK1 or HPRT.

Antibodies which may be adapted to generate extracellular domains of a chimeric receptor are well known in the art and are commercially available. Examples of commercially available antibodies include, but are not limited to: anti-HGPRT, clone 13H11.1 (EMD Millipore), anti-ROR1 (ab135669) (Abcam), anti-MUC1 [EP1024Y] (ab45167) (Abcam), anti-MUC16 [X75] (ab1107) (Abcam), anti-EGFRvIII [L8A4] (Absolute antibody), anti-Mesothelin [EPR2685 (2)] (ab134109) (Abcam), HER2 [3B5] (ab16901) (Abcam), anti-CEA (LS-C84299-1000) (LifeSpan BioSciences), anti-BCMA (ab5972) (Abcam), anti-Glypican 3 [9C2] (ab129381) (Abcam), anti-FAP (ab53066) (Abcam), anti-EphA2 [RM-0051-8F21] (ab73254) (Abcam), anti-GD2 (LS-C546315) (LifeSpan BioSciences), anti-CD19 [2E2B6B10] (ab31947) (Abcam), anti-CD20 [EP459Y] (ab78237) (Abcam), anti-CD30 [EPR4102] (ab134080) (Abcam), anti-CD33 [SP266] (ab199432) (Abcam), anti-CD123 (ab53698) (Abcam), anti-CD133 (BioLegend), anti-CD123 (1A3H4) ab181789 (Abcam), and anti-CD171 (L1.1) (Invitrogen antibodies). Techniques for creating antibody fragments, such as single-chain variable fragments (scFvs), from known antibodies are routine in the art. Further, generating sequences encoding such fragments and recombinantly including them in as part of a polynucleotide encoding a chimeric protein is also routine in the art.

In certain embodiments any ligand that has been shown to be cancer-associated or cancer-specific either due to upregulation or mutation could be used in the present disclosure.

In certain embodiments, the extracellular domain may comprise an antibody or a fragment there of that binds to a ligand. Examples of antibodies and fragments thereof include, but are not limited to IgAs, IgDs, IgEs, IgGs, IgMs, Fab fragments, F(ab′); fragments, monovalent antibodies, ScFv fragments, scRv-Fc fragments, IgNARs, hcIgGs, VhH antibodies, nanobodies, and alphabodies. In additional embodiments, the extracellular domain may comprise any amino acid sequence that allows for the specific binding of a ligand, including, but not limited to, dimerization domains, receptors, binding pockets, etc.

In embodiments, the chimeric receptor may contain a hinge region. Without limitation, the hinge region may be located between the extracellular domain and the transmembrane domain of the chimeric receptor. Without limitation, the linker may be a leucine rich repeat (LRR), or a hinge region from a toll-like receptor, an IgG, IgG4, CD8m or FcγIIIa-hinge. In embodiments, cysteines in the hinge region may be replaced with serines. Other examples of hinge regions are well known in the art.

Chimeric receptors as described herein may comprise one or more of SEQ ID Nos: 1, 3, 4, 5, 7, 9, 11, 13, 15, 17, 19, 21, 23, and 25-34, fragments of any of thereof, and/or polypeptides having at least 90% sequence identity to at least one of SEQ ID Nos 1, 3, 4, 5, 7, 9, 11, 13, 15, 17, 19, 21, 23, and 25-34 or fragments thereof. Examples of chimeric receptors include, but are not limited to, SEQ ID Nos: 35-54, or a homologue or fragment thereof. In another embodiment, the polypeptide comprises an amino acid sequence selected from the group consisting of a polypeptide having at least 90% sequence identity to at least one of SEQ ID Nos 35-54.

Embodiments include nucleic acid sequences comprising a nucleic acid sequence encoding a chimeric receptor as described above. Examples of such nucleic acids may comprise one or more of SEQ ID Nos: 2, 6, 8, 10, 12, 14, 16, 18, 20, 22, and 24, fragments of any of thereof, and/or nucleic acids having at least 90% sequence identity to at least one of SEQ ID Nos 2, 6, 8, 10, 12, 14, 16, 18, 20, 22, and 24 or fragments thereof. Further examples include nucleic acids encoding one or more of SEQ ID Nos: 24-54 and fragments of any of thereof.

In embodiments, the chimeric receptors may be glycosylated, pegylated, and/or otherwise post-translationally modified. Further, the nucleic acid sequence may be part of a vector. By way of example, the vector may be a plasmid, phage, cosmid, artificial chromosome, viral vector, AAV vector, adenoviral vector, or lentiviral vector. In certain embodiments, a nucleic acid encoding a chimeric receptor may be operably linked to a promoter and/or other regulatory sequences (e.g. enhancers, silencers, insulators, locus control regions, cis-acting elements, etc.). A non-limiting list of example promoters includes, CD68 promoter (for example CD68/150(r)), EF1α, CMV, lysM, csflr, CD11c, SRA, and CDF11b.

### Nucleotide Sequences Encoding a CAR

Further embodiments include cells comprising a chimeric receptor or nucleic acids encoding a chimeric receptor. Non-limiting examples of such cells include myeloid cells, myeloid progenitor cells, monocytes, neutrophils, basophils, eosinophils, megakaryocytes, T cells, B cells, natural killer cells, leukocytes, lymphocytes, dendritic cells, and macrophages.

Embodiments include methods of polarizing a macrophage by contacting a macrophage comprising a chimeric receptor with a ligand for the extracellular domain of the chimeric receptor; binding the ligand to the extracellular domain of the chimeric receptor. The binding of the ligand to the extracellular domain of the chimeric receptor activates the cytoplasmic portion and the activation of the cytoplasmic portion polarizes the macrophage.

Nucleotide, polynucleotide, or nucleic acid sequence will be understood according to the present disclosure as meaning both a double-stranded or single-stranded DNA or RNA in the monomeric and dimeric (so-called in tandem) forms and the transcription products of said DNAs or RNAs. In some embodiments the DNA includes a nuclear localization protein attached or associated with the DNA to aid in the translocation to the nucleus for expression.

Aspects of the disclosure relate nucleotide sequences which it has been possible to isolate, purify or partially purify, starting from separation methods such as, for example, ion-exchange chromatography, by exclusion based on molecular size, or by affinity, or alternatively fractionation techniques based on solubility in different solvents, or starting from methods of genetic engineering such as amplification, cloning, and subcloning, it being possible for the sequences to be carried by vectors.

A nucleotide sequence fragment will be understood as designating any nucleotide fragment, and may include, by way of non-limiting examples, length of at least 8, 12, 20, 25, 50, 75, 100, 200, 300, 400, 500, 1000, or more, consecutive nucleotides of the sequence from which it originates.

A specific fragment of a nucleotide sequence will be understood as designating any nucleotide fragment of, having, after alignment and comparison with the corresponding wild-type sequence, at least one less nucleotide or base.

Homologous nucleotide sequence as used herein is understood as meaning a nucleotide sequence having at least a percentage identity with the bases of a nucleotide sequence of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7%, this percentage being purely statistical and it being possible to distribute the differences between the two nucleotide sequences at random and over the whole of their length.

Specific homologous nucleotide sequences in the sense of the present disclosure is understood as meaning a homologous sequence having at least one sequence of a specific fragment, such as defined above. Said “specific” homologous sequences can comprise, for example, the sequences corresponding to a genomic sequence or to the sequences of its fragments representative of variants of the genomic sequence. These specific homologous sequences can thus correspond to variations linked to mutations within the sequence and especially correspond to truncations, substitutions, deletions and/or additions of at least one nucleotide. Said homologous sequences can likewise correspond to variations linked to the degeneracy of the genetic code.

The term “degree or percentage of sequence homology” refers to “degree or percentage of sequence identity between two sequences after optimal alignment” as defined in the present application.

Two nucleotide sequences are said to be “identical” if the sequence of amino-acids or nucleotidic residues, in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2:482 (1981), by the homology alignment algorithm of Neddleman and Wunsch, J. Mol. Biol. 48:443 (1970), by the search for similarity method of Pearson and Lipman, Proc. Natl. Acad. Sci. (U.S.A.) 85:2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection.

“Percentage of sequence identity” (or degree of identity) is determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

The definition of sequence identity given above is the definition that would be used by one of skill in the art. The definition by itself does not need the help of any algorithm, said algorithms being helpful only to achieve the optimal alignments of sequences, rather than the calculation of sequence identity.

From the definition given above, it follows that there is a well defined and only one value for the sequence identity between two compared sequences which value corresponds to the value obtained for the best or optimal alignment.

In the BLAST N or BLAST P “BLAST 2 sequence”, software which is available in the web site worldwideweb.ncbi.nlm.nih.gov/gorf/bl2.html, and habitually used by the inventors and in general by the skilled person for comparing and determining the identity between two sequences, gap cost which depends on the sequence length to be compared is directly selected by the software (i.e. 11.2 for substitution matrix BLOSUM-62 for length>85).

Complementary nucleotide sequence of a sequence as used herein is understood as meaning any DNA whose nucleotides are complementary to those of the sequences and whose orientation is reversed (antisense sequence).

Hybridization under conditions of stringency with a nucleotide sequence as used herein is understood as meaning hybridization under conditions of temperature and ionic strength chosen in such a way that they allow the maintenance of the hybridization between two fragments of complementary DNA.

By way of illustration, conditions of great stringency of the hybridization step with the aim of defining the nucleotide fragments described above are advantageously the following.

The hybridization is carried out at a preferential temperature of 65° C. in the presence of SSC buffer, 1×SSC corresponding to 0.15 M NaCl and 0.05 M Na citrate. The washing steps, for example, can be the following: 2×SSC, at ambient temperature followed by two washes with 2×SSC. 0.5% SDS at 65° C.; 2×0.5×SSC, 0.5% SDS; at 65° C. for 10 minutes each.

The conditions of intermediate stringency, using, for example, a temperature of 42° C. in the presence of a 2×SSC buffer, or of less stringency, for example a temperature of 37° C. in the presence of a 2×SSC buffer, respectively require a globally less significant complementarity for the hybridization between the two sequences.

The stringent hybridization conditions described above for a polynucleotide with a size of approximately 350 bases will be adapted by the person skilled in the art for oligonucleotides of greater or smaller size, according to the teaching of Sambrook et al., 1989.

Among the nucleotide sequences described herein, are those which can be used as a primer or probe in methods allowing the homologous sequences to be obtained, these methods, such as the polymerase chain reaction (PCR), nucleic acid cloning, and sequencing, being well known to the person skilled in the art.

Among the nucleotide sequences are those which can be used as a primer or probe in methods allowing the presence of specific nucleic acids, one of their fragments, or one of their variants such as defined below to be determined. In embodiments, the nucleotide sequences may comprise fragments of SEQ ID Nos. 2, 6, 8, 10, 12, 14, 16, 18, 20, 22, and 24 which encode a transmembrane domain, cytosolic domain, or a portion thereof. Further fragments may include nucleotide sequences encoding linkers, hinges, or fragments thereof such as nucleotides encoding one or more of SEQ ID NOS: 26-34. Further fragments may include fragments of nucleotide sequences encoding one or more of SEQ ID NOs: 35-54.

The nucleotide sequence fragments can be obtained, for example, by specific amplification, such as PCR, or after digestion with appropriate restriction enzymes of nucleotide sequences, these methods in particular being described in the work of Sambrook et al., 1989. Also, such fragments may be obtained with gene synthesis standard technology available from companies such as Genescript. Such representative fragments can likewise be obtained by chemical synthesis according to methods well known to persons of ordinary skill in the art.

Modified nucleotide sequence will be understood as meaning any nucleotide sequence obtained by mutagenesis according to techniques well known to the person skilled in the art, and containing modifications with respect to a wild-type sequence, for example mutations in the regulatory and/or promoter sequences of polypeptide expression, especially leading to a modification of the rate of expression of said polypeptide or to a modulation of the replicative cycle.

Modified nucleotide sequence will likewise be understood as meaning any nucleotide sequence coding for a modified polypeptide such as defined below.

Disclosed are nucleotide sequences encoding a chimeric receptor, the nucleotide sequences comprising nucleotide sequences selected from SEQ ID Nos. 2, 6, 8, 10, 12, 14, 16, 18, 20, 22, and 24 or one of their fragments. Such fragments may encode particular domains such as transmembrane domains or cytosolic domains or portions thereof. Further nucleotide sequences encoding a chimeric receptor may include nucleotide sequences encoding linkers, hinges, or fragments thereof such as nucleotides encoding one or more of SEQ ID NOs: 26-34. Nucleotide sequences encoding a chimeric receptor may further nucleotide sequences encoding one or more of SEQ ID NOs: 35-54 or fragments thereof.

Embodiments likewise relate to nucleotide sequences characterized in that they comprise a nucleotide sequence selected from: a) at least one of a nucleotide sequence of SEQ ID Nos. 2, 6, 8, 10, 12, 14, 16, 18, 20, 22, and 24, a nucleotide sequence encoding at least one of SEQ ID NOs: 25-54, or one of their fragments; b) a nucleotide sequence of a specific fragment of a sequence such as defined in a); c) a homologous nucleotide sequence having at least 80% identity with a sequence such as defined in a) or b); d) a complementary nucleotide sequence or sequence of RNA corresponding to a sequence such as defined in a), b) or c); and c) a nucleotide sequence modified by a sequence such as defined in a), b), c) or d).

Among the nucleotide sequences are the nucleotide sequences of SEQ ID Nos. 2, 6, 8, 10, 12, 14, 16, 18, 20, 22, and 24, a nucleotide sequence encoding at least one of SEQ ID NOs: 25-54, or fragments thereof and any nucleotide sequences which have a homology of at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7% identity with the at least one of the sequences of SEQ ID Nos. 2, 6, 8, 10, 12, 14, 16, 18, 20, 22, and 24 a nucleotide sequence encoding at least one of SEQ ID NOs: 25-54, or fragments thereof. Said homologous sequences can comprise, for example, the sequences corresponding to the wild-type sequences. In the same manner, these specific homologous sequences can correspond to variations linked to mutations within the wild-type sequence and especially correspond to truncations, substitutions, deletions and/or additions of at least one nucleotide. As will be apparent to one of ordinary skill in the art, such homologues are easily created and identified using standard techniques and publicly available computer programs such as BLAST. As such, each homologue referenced above should be considered as set forth herein and fully described.

Embodiments comprise the chimeric receptors coded for by a nucleotide sequence described herein, or fragments thereof, whose sequence is represented by a fragment. Amino acid sequences corresponding to the polypeptides which can be coded for according to one of the three possible reading frames of at least one of the sequences of SEQ ID Nos. 35-54.

Embodiments likewise relate to chimeric receptors, characterized in that they comprise a polypeptide selected from at least one of the amino acid sequences of SEQ ID Nos. 1, 305, 7, 9, 11, 13, 15, 17, 19, 21, 23, and 25-54, or one of their fragments.

Among the polypeptides, according to embodiments, are the polypeptides of amino acid sequence SEQ ID Nos., 3, 5, 7, 9, 11, 13, 15, 17, 19, 21, 23, and 25-54, or fragments thereof or any other polypeptides which have a homology of at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7% identity with at least one of the sequences of SEQ ID Nos. 1, 305, 7, 9, 11, 13, 15, 17, 19, 21, 23, and 25-54 or fragments thereof. As will be apparent to one of ordinary skill in the art, such homologues are easily created and identified using standard techniques and publicly available computer programs such as BLAST. As such, each homologue referenced above should be considered as set forth herein and fully described.

### Polypeptides Encoded by the Nucleic Acid

Embodiments also relate to the polypeptides, characterized in that they comprise a polypeptide selected from: a) a specific fragment of at least 5 amino acids of a polypeptide of an amino acid sequence; b) a polypeptide homologous to a polypeptide such as defined in a); c) a specific biologically active fragment of a polypeptide such as defined in a) or b); and d) a polypeptide modified by a polypeptide such as defined in a), b) or c).

In the present description, the terms polypeptide, peptide and protein are interchangeable.

In embodiments, the chimeric receptors may be glycosylated, pegylated, and/or otherwise post-translationally modified. In further embodiments, glycosylation, pegylation, and/or other posttranslational modifications may occur in vivo or in vitro and/or may be performed using chemical techniques. In additional embodiments, any glycosylation, pegylation and/or other posttranslational modifications may be N-linked or O-linked.

In embodiments any one of the chimeric receptors may be enzymatically or functionally active such that, when the extracellular domain is bound by a ligand, a signal is transduced to polarize a macrophage.

As used herein, a “polarized macrophage” is a macrophage that correlates with an M1 or M2 macrophage phenotype. M1 polarized macrophages secrete IL-12 and IL-23. The determination of a macrophage as polarized to M1 may be performed by measuring the expression of TNF-α, IL-12, and/or IL-23 using a standard cytokine assay and comparing that expression to the expression by newly differentiated unpolarized macrophages. Alternatively, the determination can be made by determining if the cells are CD14+, CD80+, CD206+, and CDCD163−. M2 polarized macrophages secrete IL-10. The determination of a macrophage as polarized to M2 may be performed by measuring the expression of IL-10 using a standard cytokine assay and comparing that expression to the expression by newly differentiated unpolarized macrophages. Alternatively, the determination can be made by determining if the cells are CD14+, CD80−, CD206+, and CDCD163+.

### Genetic Recombination

Aspects of the disclosure relate to chimeric receptors obtained by genetic recombination, or alternatively by chemical synthesis and that they may thus contain unnatural amino acids, as will be described below.

A “polypeptide fragment” according to the embodiments is understood as designating a polypeptide containing at least 5 consecutive amino acids, preferably 10 consecutive amino acids or 15 consecutive amino acids.

Herein, a specific polypeptide fragment is understood as designating the consecutive polypeptide fragment coded for by a specific fragment a nucleotide sequence.

“Homologous polypeptide” will be understood as designating the polypeptides having. with respect to the natural polypeptide, certain modifications such as, in particular, a deletion, addition, or substitution of at least one amino acid, a truncation, a prolongation, a chimeric fusion, and/or a mutation. Among the homologous polypeptides, those are preferred whose amino acid sequence has at least 80% or 90%, homology with the sequences of amino acids of polypeptides described herein.

“Specific homologous polypeptide” will be understood as designating the homologous polypeptides such as defined above and having a specific fragment of polypeptide polypeptides described herein.

In the case of a substitution, one or more consecutive or nonconsecutive amino acids are replaced by “equivalent” amino acids. The expression “equivalent” amino acid is directed here at designating any amino acid capable of being substituted by one of the amino acids of the base structure without, however, essentially modifying the biological activities of the corresponding peptides and such that they will be defined by the following. As will be apparent to one of ordinary skill in the art, such substitutions are easily created and identified using standard molecular biology techniques and publicly available computer programs such as BLAST. As such, each substitution referenced above should be considered as set forth herein and fully described.

These equivalent amino acids can be determined either by depending on their structural homology with the amino acids which they substitute, or on results of comparative tests of biological activity between the different polypeptides, which are capable of being carried out.

By way of nonlimiting example, the possibilities of substitutions capable of being carried out without resulting in an extensive modification of the biological activity of the corresponding modified polypeptides will be mentioned, the replacement, for example, of leucine by valine or isoleucine, of aspartic acid by glutamic acid, of glutamine by asparagine, of arginine by lysine etc., the reverse substitutions naturally being envisageable under the same conditions.

In a further embodiment, substitutions are limited to substitutions in amino acids not conserved among other proteins which have similar identified enzymatic activity. For example, one of ordinary skill in the art may align proteins of the same function in similar organisms and determine which amino acids are generally conserved among proteins of that function. One example of a program that may be used to generate such alignments is wordlwideweb.charite.de/bioinf/strap/in conjunction with the databases provided by the NCBI.

Thus, according to one embodiment, substitutions or mutation may be made at positions that are generally conserved among proteins of that function. In a further embodiment, nucleic acid sequences may be mutated or substituted such that the amino acid they code for is unchanged (degenerate substitutions and/mutations) and/or mutated or substituted such that any resulting amino acid substitutions or mutation are made at positions that are generally conserved among proteins of that function.

The specific homologous polypeptides likewise correspond to polypeptides coded for by the specific homologous nucleotide sequences such as defined above and thus comprise in the present definition the polypeptides which are mutated or correspond to variants which can exist in wild-type sequences, and which especially correspond to truncations, substitutions, deletions, and/or additions of at least one amino acid residue.

“Specific biologically active fragment of a polypeptide” as used herein will be understood in particular as designating a specific polypeptide fragment, such as defined above, having at least one of the characteristics of polypeptides described herein. In certain embodiments the peptide is capable of behaving as chimeric antigen receptor that when activated polarizes a macrophage.

“Modified polypeptide” of a polypeptide as used herein is understood as designating a polypeptide obtained by genetic recombination or by chemical synthesis as will be described below, having at least one modification with respect to a wild-type sequence. These modifications may or may not be able to bear on amino acids at the origin of specificity, and/or of activity, or at the origin of the structural conformation, localization, and of the capacity of membrane insertion of the polypeptide as described herein. It will thus be possible to create polypeptides of equivalent, increased, or decreased activity, and of equivalent, narrower, or wider specificity. Among the modified polypeptides, it is necessary to mention the polypeptides in which up to 5 or more amino acids can be modified, truncated at the N- or C-terminal end, or even deleted or added.

The methods allowing said modulations on eukaryotic or prokaryotic cells to be demonstrated are well known to the person of ordinary skill in the art. It is likewise well understood that it will be possible to use the nucleotide sequences coding for said modified polypeptides for said modulations, for example through vectors and described below.

The preceding modified polypeptides can be obtained by using combinatorial chemistry, in which it is possible to systematically vary parts of the polypeptide before testing them on models, cell cultures or microorganisms for example, to select the compounds which are most active or have the properties sought.

Chemical synthesis likewise has the advantage of being able to use unnatural amino acids, or nonpeptide bonds.

Thus, in order to improve the duration of life of the polypeptides, it may be of interest to use unnatural amino acids, for example in D form, or else amino acid analogs, especially sulfur-containing forms, for example.

Finally, it will be possible to integrate the structure of the polypeptides, its specific or modified homologous forms, into chemical structures of polypeptide type or others. Thus, it may be of interest to provide at the N- and C-terminal ends compounds not recognized by proteases.

### Nucleotide Sequences Encoding a Polypeptide

The nucleotide sequences coding for a polypeptide are likewise disclosed herein.

Embodiments likewise relates to nucleotide sequences utilizable as a primer or probe, characterized in that said sequences are selected from the nucleotide sequences described herein.

It is well understood that various embodiments likewise relate to specific polypeptides including chimeric receptors, coded for by nucleotide sequences, capable of being obtained by purification from natural polypeptides, by genetic recombination or by chemical synthesis by procedures well known to the person skilled in the art and such as described below. In the same manner, the labeled or unlabeled mono- or polyclonal antibodies directed against said specific polypeptides coded for by said nucleotide sequences are also encompassed by this disclosure.

Embodiments additionally relate to the use of a nucleotide sequence as a primer or probe for the detection and/or the amplification of nucleic acid sequences.

The nucleotide sequences according to embodiments can thus be used to amplify nucleotide sequences, especially by the PCR technique (polymerase chain reaction) (Erlich, 1989; Innis et al., 1990; Rolfs et al., 1991; and White et al., 1997).

These oligodeoxyribonucleotide or oligoribonucleotide primers advantageously have a length of at least 8 nucleotides, preferably of at least 12 nucleotides, and even more preferentially at least 20 nucleotides.

Other amplification techniques of the target nucleic acid can be advantageously employed as alternatives to PCR.

The nucleotide sequences described herein, in particular the primers, can likewise be employed in other procedures of amplification of a target nucleic acid, such as: the TAS technique (Transcription-based Amplification System), described by Kwoh et al. in 1989; the 3SR technique (Self-Sustained Sequence Replication), described by Guatelli et al. in 1990; the NASBA technique (Nucleic Acid Sequence Based Amplification), described by Kievitis et al. in 1991; the SDA technique (Strand Displacement Amplification) (Walker et al., 1992); the TMA technique (Transcription Mediated Amplification).

The polynucleotides, including chimeric receptors, can also be employed in techniques of amplification or of modification of the nucleic acid serving as a probe, such as: the LCR technique (Ligase Chain Reaction), described by Landegren et al. in 1988 and improved by Barany et al. in 1991, which employs a thermostable ligase; the RCR technique (Repair Chain Reaction), described by Segev in 1992; the CPR technique (Cycling Probe Reaction), described by Duck et al. in 1990; the amplification technique with Q-beta replicase, described by Miele et al. in 1983 and especially improved by Chu et al. in 1986, Lizardi et al. in 1988, then by Burg et al. as well as by Stone et al. in 1996.

In the case where the target polynucleotide to be detected is possibly an RNA, for example an mRNA, it will be possible to use, prior to the employment of an amplification reaction with the aid of at least one primer or to the employment of a detection procedure with the aid of at least one probe, an enzyme of reverse transcriptase type in order to obtain a cDNA from the RNA contained in the biological sample. The cDNA obtained will thus serve as a target for the primer(s) or the probe(s) employed in the amplification or detection procedure.

The detection probe will be chosen in such a manner that it hybridizes with the target sequence or the amplicon generated from the target sequence. By way of sequence, such a probe will advantageously have a sequence of at least 12 nucleotides, in particular of at least 20 nucleotides, and preferably of at least 100 nucleotides.

Embodiments also comprise the nucleotide sequences utilizable as a probe or primer, characterized in that they are labeled with a radioactive compound or with a nonradioactive compound.

The unlabeled nucleotide sequences can be used directly as probes or primers, although the sequences are generally labeled with a radioactive isotope (32P, 35S, 3H, 1251) or with a nonradioactive molecule (biotin, acetylaminofluorene, digoxigenin, 5-bromodeoxyuridine, fluorescein) to obtain probes which are utilizable for numerous applications.

Examples of nonradioactive labeling of nucleotide sequences are described, for example, in French Patent No. 78.10975 or by Urdea et al. or by Sanchez-Pescador et al. in 1988.

In the latter case, it will also be possible to use one of the labeling methods described in patents FR-2 422 956 and FR-2 518 755.

The hybridization technique can be carried out in various manners (Matthews et al., 1988). The most general method consists in immobilizing the nucleic acid extract of cells on a support (such as nitrocellulose, nylon, polystyrene) and in incubating, under well-defined conditions, the immobilized target nucleic acid with the probe. After hybridization, the excess of probe is eliminated and the hybrid molecules formed are detected by the appropriate method (measurement of the radioactivity, of the fluorescence or of the enzymatic activity linked to the probe).

Various embodiments likewise comprise the nucleotide sequences or polypeptide sequences described herein, characterized in that they are immobilized on a support, covalently or noncovalently.

According to another advantageous mode of employing nucleotide sequences, the latter can be used immobilized on a support and can thus serve to capture, by specific hybridization, the target nucleic acid obtained from the biological sample to be tested. If necessary, the solid support is separated from the sample and the hybridization complex formed between said capture probe and the target nucleic acid is then detected with the aid of a second probe, a so-called detection probe, labeled with an easily detectable element.

Another aspect is a vector for the cloning and/or expression of a sequence, characterized in that it contains a nucleotide sequence described herein.

The vectors, characterized in that they contain the elements allowing the integration, expression and/or the secretion of said nucleotide sequences in a determined host cell, are likewise provided.

The vector may then contain a promoter, signals of initiation and termination of translation, as well as appropriate regions of regulation of transcription. It may be able to be maintained stably in the host cell and can optionally have particular signals specifying the secretion of the translated protein. These different elements may be chosen as a function of the host cell used. To this end, the nucleotide sequences described herein may be inserted into autonomous replication vectors within the chosen host, or integrated vectors of the chosen host.

Such vectors will be prepared according to the methods currently used by the person skilled in the art, and it will be possible to introduce the clones resulting therefrom into an appropriate host by standard methods, such as, for example, calcium phosphate prescipitation, lipofection, electroporation, and thermal shock.

The vectors according are, for example, vectors of plasmid or viral origin. Examples of vectors for the expression of polypeptides described herein are plasmids, phages, cosmids, artificial chromosomes, viral vectors, AAV vectors, baculovirus vectors, adenoviral vectors, lentiviral vectors, retroviral vectors, chimeric viral vectors, and chimeric adenoviridae such as AD5/F35.

These vectors are useful for transforming host cells in order to clone or to express the nucleotide sequences described herein.

### The Transformed Cells

Embodiments likewise comprise the host cells transformed by a vector.

These cells can be obtained by the introduction into host cells of a nucleotide sequence inserted into a vector such as defined above, then the culturing of said cells under conditions allowing the replication and/or expression of the transfected nucleotide sequence.

The host cell can be selected from prokaryotic or eukaryotic systems, such as, for example, bacterial cells (Olins and Lee, 1993), but likewise yeast cells (Buckholz, 1993), as well as plants cells, such as Arabidopsis sp., and animal cells, in particular the cultures of mammalian cells (Edwards and Aruffo, 1993), for example, HEK 293, cells, HEK 293T cells, Chinese hamster ovary (CHO) cells myeloid cells, myeloid progenitor cells, monocytes, neutrophils, basophils, cosinophils, megakaryocytes, T cells, B cells, natural killer cells, leukocytes, lymphocytes, dendritic cells, and macrophages, but likewise the cells of insects in which it is possible to use procedures employing baculoviruses, for example sf9 insect cells (Luckow, 1993).

Embodiments likewise relate to organisms comprising one of said transformed cells.

The obtainment of transgenic organisms expressing one or more of the nucleic acids or part of the nucleic acids may be carried out in, for example, rats, mice, or rabbits according to methods well known to the person skilled in the art, such as by viral or nonviral transfections. It will be possible to obtain the transgenic organisms expressing one or more of said genes by transfection of multiple copies of said genes under the control of a strong promoter of ubiquitous nature, or selective for one type of tissue. It will likewise be possible to obtain the transgenic organisms by homologous recombination in embryonic cell strains, transfer of these cell strains to embryos, selection of the affected chimeras at the level of the reproductive lines, and growth of said chimeras.

The transformed cells as well as the transgenic organisms are utilizable in procedures for preparation of recombinant polypeptides.

It is today possible to produce recombinant polypeptides in relatively large quantity by genetic engineering using the cells transformed by expression vectors or using transgenic organisms.

The procedures for preparation of a polypeptide, such as a chimeric receptor, in recombinant form, characterized in that they employ a vector and/or a cell transformed by a vector and/or a transgenic organism comprising one of said transformed cells are themselves comprised in in the present disclosure.

As used herein, “transduce,” “transfect,” “transform,” “transformation,” and “transformed”, as well as the various tenses and participles thereof relate to the introduction of nucleic acids into a cell, whether prokaryotic or eukaryotic, by any means or method and not meant to refer to or be limited to any particular method of introduction. As such, the terms can refer to any or all of delivery of nucleic acid to a cell by any method such as, nanoparticle, liposome, transfectamine, virus, VLP, nucleofection, or electroporation Further, “transformation” and “transformed,” as used herein, need not relate to growth control or growth deregulation.

Among said procedures for preparation of a polypeptide, such as a chimeric receptor, in recombinant form, the preparation procedures employing a vector, and/or a cell transformed by said vector and/or a transgenic organism comprising one of said transformed cells, containing a nucleotide sequence, such as those encoding a chimeric receptor.

A variant according, as used herein, may consist of producing a recombinant polypeptide fused to a “carrier” protein (chimeric protein). The advantage of this system is that it may allow stabilization of and/or a decrease in the proteolysis of the recombinant product, an increase in the solubility in the course of renaturation in vitro and/or a simplification of the purification when the fusion partner has an affinity for a specific ligand.

### Preparing the Polypeptide

More particularly, embodiments relate to a procedure for preparation of a polypeptide comprising the following steps: a) culture of transformed cells under conditions allowing the expression of a recombinant polypeptide of nucleotide sequence; b) if need be, recovery of said recombinant polypeptide.

When the procedure for preparation of a polypeptide, such as a chimeric receptor, employs a transgenic organism, the recombinant polypeptide may then extracted from said organism or left in place.

Embodiments also relate to a polypeptide which is capable of being obtained by a procedure such as described previously.

Embodiments also comprise a procedure for preparation of a synthetic polypeptide, characterized in that it uses a sequence of amino acids of polypeptides.

This disclosure likewise relates to a synthetic polypeptide, such as a chimeric receptor, obtained by a procedure.

The polypeptides, such as chimeric receptors, can likewise be prepared by techniques which are conventional in the field of the synthesis of peptides. This synthesis can be carried out in homogeneous solution or in solid phase.

For example, recourse can be made to the technique of synthesis in homogeneous solution described by Houben-Weyl in 1974.

This method of synthesis consists in successively condensing, two by two, the successive amino acids in the order required, or in condensing amino acids and fragments formed previously and already containing several amino acids in the appropriate order, or alternatively several fragments previously prepared in this way, it being understood that it will be necessary to protect beforehand all the reactive functions carried by these amino acids or fragments, with the exception of amine functions of one and carboxyls of the other or vice-versa, which must normally be involved in the formation of peptide bonds, especially after activation of the carboxyl function, according to the methods well known in the synthesis of peptides.

Recourse may also be made to the technique described by Merrifield.

To make a peptide chain according to the Merrifield procedure, recourse is made to a very porous polymeric resin, on which is immobilized the first C-terminal amino acid of the chain. This amino acid is immobilized on a resin through its carboxyl group and its amine function is protected. The amino acids which are going to form the peptide chain are thus immobilized, one after the other, on the amino group, which is deprotected beforehand each time, of the portion of the peptide chain already formed, and which is attached to the resin. When the whole of the desired peptide chain has been formed, the protective groups of the different amino acids forming the peptide chain are eliminated and the peptide is detached from the resin with the aid of an acid.

These hybrid molecules can be formed, in part, of a polypeptide carrier molecule or of fragments thereof, associated with a possibly immunogenic part, in particular an epitope of the diphtheria toxin, the tetanus toxin, a surface antigen of the hepatitis B virus (patent FR 79 21811), the VP1 antigen of the poliomyelitis virus or any other viral or bacterial toxin or antigen.

The polypeptides, including chimeric receptors, the antibodies described below and the nucleotide sequences encoding any of the foregoing can advantageously be employed in procedures for the polarization of a macrophage.

In embodiments, a nucleic acid sequence encoding a chimeric receptor is provided to a cell. The cell may then express the encoded chimeric receptor. The expressed chimeric receptor may be present on the surface of the cell or in the cytoplasm. In particular embodiments, the cell expressing the chimeric receptor is a macrophage. The macrophage expressed chimeric receptor may bind a ligand. and binding of the ligand may activate the chimeric receptor so as to induce polarization of the macrophage as previously described.

In embodiments, the cell provided with the nucleic acid sequence encoding a chimeric receptor may be isolated from a subject. After the cell is provided with the nucleic acid, the cell may be returned to the subject from whom it was obtained, for example by injection or transfusion. In other embodiments, the cell provided with the nucleic acid may be provided by a donor. After the donor cell is provided with the nucleic acid, the cell may then be provided to an individual other than the donor. Examples of donor cells include but are not limited to primary cells from a subject and cells from a cell line.

In other embodiments, chimeric receptors may be introduced directly into cells. Any method of introducing a protein into cell may be used, including, but not limited to, microinjection, electroporation, membrane fusion, and the use of protein transduction domains. After the cell is provided with chimeric receptors, the cell may be returned to the subject from whom it was obtained, for example by injection or transfusion. In other embodiments, the cell provided with the chimeric receptors be provided by a donor. After the donor cell is provided with the nucleic acid, the cell may then be provided to an individual other than the donor. Examples of donor cells include, but are not limited to primary cells from a subject and cells from a cell line.

### Labeled Polypeptides

Embodiments likewise relates to polypeptides, such as chimeric receptors, labeled with the aid of an adequate label, such as, of the enzymatic, fluorescent or radioactive type.

The polypeptides allow monoclonal or polyclonal antibodies to be prepared which are characterized in that they specifically recognize the polypeptide. It will advantageously be possible to prepare the monoclonal antibodies from hybridomas according to the technique described by Kohler and Milstein in 1975. It will be possible to prepare the polyclonal antibodies, for example, by immunization of an animal, in particular a mouse, with a polypeptide or a DNA, associated with an adjuvant of the immune response, and then purification of the specific antibodies contained in the serum of the immunized animals on an affinity column on which the polypeptide which has served as an antigen has previously been immobilized. The polyclonal antibodies can also be prepared by purification, on an affinity column on which a polypeptide has previously been immobilized, of the antibodies contained in the serum of an animal immunologically challenged by a chimeric receptor, or a polypeptide or fragment thereof.

In addition, antibodies can be used to prepare other forms of binding molecules, including, but not limited to, IgAs, IgDs, IgEs, IgGs, IgMs, Fab fragments, F(ab′)₂ fragments, monovalent antibodies, scFv fragments, scRv-Fc fragments, IgNARs, hcIgGs, VhH antibodies, nanobodies, and alphabodies.

Embodiments likewise relates to mono- or polyclonal antibodies or their fragments, or chimeric antibodies, or fragments thereof, characterized in that they are capable of specifically recognizing a polypeptide described herein or a ligand of a polypeptide and/or chimeric recpetor.

It will likewise be possible for the antibodies to be labeled in the same manner as described previously for the nucleic probes, such as a labeling of enzymatic, fluorescent or radioactive type. It will be also be possible to include such antibodies and/or fragments thereof as part of a chimeric receptor. By way of non-limiting example, such an antibody or fragment thereof may make up a portion of the extracellular domain of a chimeric receptor.

Embodiments are additionally directed at a procedure for the detection and/or identification of chimeric receptor in a sample, characterized in that it comprises the following steps: a) contacting of the sample with a mono- or polyclonal (under conditions allowing an immunological reaction between said antibodies and the chimeric receptor possibly present in the biological sample); b) demonstration of the antigen-antibody complex possibly formed.

### Monocytes and Cancer

It is understood that monocytes display diverse function at different stages of tumor growth and progression. In some instances, monocytes may perform opposing roles due to differences in cancer type and tissue origin, subtle differences in tumor microenvironment, and stage of tumor growth. Monocytes are recruited throughout tumor progression, including during early stages of tumor growth and establishment of distal metasteses.

Monocytes appear to have the cellular machinery to directly kill malignant cells by cytokine-mediated induction of cell death and phagocytosis. Most tumoricidal activity has been demonstrated in vitro, thus whether monocyte-mediated killing is part of the in vivo antitumoral response during cancer progression needs further exploration. Peripheral blood monocytes exposed to IFN-γ or IFN-α produce the protein TRAIL, which is able to induce cell death in TRAIL-sensitive cancer cells. However, many cancer cells are resistant to TRAIL-mediated apoptosis and TRAIL can instead stimulate secretion of protumoral cytokines such as CCL2 and IL-8. Monocytes can also induce cancer cell death through Ab-dependent cellular cytotoxicity, which both CD14+ and CD16+ monocyte subsets have the capacity for. CD16+ monocytes require contact with tumor cells and TNF-α signaling for induction of tumor cell cytolysis. Monocytes collected from peripheral blood or ascites fluid of human ovarian cancer patients display reduced capacity for Ab-dependent cytolysis and phagocytosis of tumor cells upon activation in vitro. In renal cell carcinoma patients, peripheral blood monocytes secrete factors that promote tumor cell invasion in vitro. Consequently, malignant cells may be able to coerce monocytes to adopt a phenotype that supports tumorigenesis, thereby overpowering any programmed tumoricidal activities.

Monocytes readily engulf bloodborne tumor-derived material such as exosomes and large microparticles, indicating that monocyte-mediated phagocytosis could be an important part of the antitumoral immune response. However, cancerous cells are able to shield themselves from phagocytosis in circulation and within primary tumor sites, for example, through expression of CD47. Circulating monocytes express high levels of SIRPα (CD172a), the ligand for CD47, but CD47/SIRPα interactions have not been extensively explored in monocyte-mediated phagocytosis of tumor cells. CD47 expression is prognostic for poor clinical outcome in a number of different cancers, including ovarian cancer, glioma, glioblastoma, and non-small cell lung cancer. Evasion of phagocytosis is likely also critical during metastasis to distal sites, as CD47 expression correlates with the presence of lymph node and distal metastases in non-small cell lung cancer patients. Whether CD47/SIRPα interactions play a role in monocyte-mediated regulation of metastasis (discussed below) also requires further investigation.

The long lifespan and patrolling activities of nonclassical monocytes make them particularly well suited to scavenge tumor cells and debris. In the context of cancer, nonclassical monocytes migrate toward metastatic sites within the lung, where they engulf tumor material and generate cytokines that regulate antitumor immunity. Tumor-derived exosomes also expand bone marrow pools of patrolling monocytes, which appears to initiate an immune surveillance cascade that prevents metastatic seeding.

Targeting and transforming monocytes that have been recruited to tumors provides a powerful therapeutic target. The targeted delivery system described in the present disclosure allows for the manipulation and control of the tumor microenvironment while not needing to directly transform the genetically unstable tumor cells. As described above, the chimeric receptor would allow for control of the polarization of the macrophage or dendritic cell from an M1 to an M2 macrophage which can be used as part of a therapeutic system for various cancers.

### Definitions

The term “transduce,” as used herein, is meant to be interchangeable with the words, “transfect.” or “transform.” and is meant to include delivery of nucleic acid to a cell by any method such as, nanoparticle, liposome, transfectamine, virus, VLP, nucleofection, or electroporation.

The terms “polynucleotide,” “nucleic acid.” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases.

“Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

Genomic DNA″ refers to the DNA of a genome of an organism including, but not limited to, the DNA of the genome of a bacterium, fungus, archacon, protist, virus, plant or animal.

The term “manipulating” DNA encompasses binding, nicking one strand, or cleaving, e.g. cutting both strands of the DNA; or encompasses modifying or editing the DNA or a polypeptide associated with the DNA. Manipulating DNA can silence, activate, or modulate (either increase or decrease) the expression of an RNA or polypeptide encoded by the DNA, or prevent or enhance the binding of a polypeptide to DNA.

By “hybridizable” or “complementary” or “substantially complementary” it is meant that a nucleic acid (e.g. RNA or DNA) includes a sequence of nucleotides that enables it to non-covalently bind, e.g., form Watson-Crick base pairs and/or G/U base pairs, “anneal”, or “hybridize.” to another nucleic acid in a sequence-specific, antiparallel, manner (e.g., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA]. In addition, it is also known in the art that for hybridization between two RNA molecules (e.g. dsRNA), guanine (G) base pairs with uracil (U). For example, G/U base-pairing is partially responsible for the degeneracy (e.g., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches becomes important (see Sambrook et al., supra, 11.7-11.8). Generally, the length for a hybridizable nucleic acid is at least 10 nucleotides. Illustrative minimum lengths for a hybridizable nucleic acid are: at least 15 nucleotides; at least 20 nucleotides; at least 22 nucleotides; at least 25 nucleotides; and at least 30 nucleotides). Furthermore, the skilled artisan will recognize that the temperature and wash solution salt concentration maybe adjusted as necessary according to factors such as length of the region of complementation and the degree of complementation.

It is understood in the art that the sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g. a loop structure or hairpin structure). A polynucleotide can include at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining non complementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol. 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math. 1981 (2) 482-489).

The terms “peptide”, “polypeptide”, and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein refers to a non-covalent interaction between macromolecules (e.g. between a protein and a nucleic acid). While in a state of non-covalent interaction, the macromolecules are said to be “associated” or “interacting” or “binding” (e.g. when a molecule X is said to interact with a molecule Y, it is meant the molecule X binds to molecule Y in a non-covalent manner). Not all components of a binding interaction need be sequence-specific (e.g. contacts with phosphate residues in a DNA backbone), but some portions of a binding interaction may be sequence-specific. “Affinity” refers to the strength of binding, increased binding affinity being correlated with a lower Kd.

By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homo-dimers, homo-trimers, etc.) and/or it can bind to one or more molecules of a different protein or proteins.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g. BLAST, T-COFFEE, MUSCLE, MAFFT, etc.), available over the world-wide-web at sites including ncbi.nlm.nili.gov/BLAST, cbi.ac.uk/Tools/msa/tcoffee, cbi.Ac.Uk/Tools/msa/muscle, mafft.cbrc/alignment/software. Sec, e.g. Altsch ul et al. (1990), J. Mol. Biol. 215:403-10. In some embodiments of the disclosure, sequence alignments standard in the art are used according to the disclosure to determine amino acid residues in M-SmallCas9 polypeptide or variant thereof that “correspond to” amino acid residues in another Cas9 endonuclease. The amino acid residues of M-SmallCas9 polypeptides or variants thereof that correspond to amino acid residues of other Cas9 endonucleases appear at the same position in alignments of the sequences.

A DNA sequence that “encodes” a particular RNA is a DNA nucleic acid sequence that is transcribed into the RNA. A polydeoxyribonucleotide may encode an RNA (mRNA) that is translated into protein, or a polydeoxyribonucleotide may encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will generally be located at 3′ of the coding sequence.

As used herein, a “promoter sequence” or “promoter” is a DNA regulatory region capable of binding RNA polymerase and initiating transcription of a downstream (3′ direction) coding or non coding sequence. As used herein, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAAT” boxes. Various promoters, including inducible promoters, may be used to drive the various vectors of the present disclosure. A promoter can be a constitutively active promoter (e.g., a promoter that is constitutively in an active “ON” state), it may be an inducible promoter (e.g., a promoter whose state, active/“ON” or inactive/“OFF”, is controlled by an external stimulus, e.g. the presence of a particular temperature, compound, or protein.), it may be a spatially restricted promoter (e.g., transcriptional control element, enhancer, etc.) (e.g. tissue specific promoter, cell type specific promoter, etc.), and it may be a temporally restricted promoter (e.g., the promoter is in the “ON” state or “OFF” state during specific stages of embryonic development or during specific stages of a biological process, e.g. hair follicle cycle in mice). Suitable promoters can be derived from viruses and can therefore be referred to as viral promoters, or they can be derived from any organism, including prokaryotic or eukaryotic organisms. Suitable promoters can be used to drive expression by any RNA polymerase (e.g. pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (FHSV) promoter, a cytomegalovirus (CMV) promoter such as the CMV immediate early promoter region (CMVIE), a Rous sarcoma virus (RSV) promoter, a human U6 small nuclear promoter (U6) (Miyagishi et al., Nature Biotechnology 20, 497-500 (2002)), an enhanced U6 promoter (e.g. Xia et al., Nucleic Acids Res. 2003 Sep. 1; 31 (17)), a human Ell promoter (HI), and the like. Examples of inducible promoters include, but are not limited to T7 RNA polymerase promoter, T3 RNA polymerase promoter, isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; RNA polymerase, e.g. T7 RNA polymerase; an estrogen receptor; an estrogen receptor fusion; etc.

In some embodiments, the promoter is a spatially restricted promoter (e.g., cell type specific promoter, tissue specific promoter, etc.) such that in a multi-cellular organism, the promoter is active (e.g., “ON”) in a subset of specific cells. Spatially restricted promoters may also be referred to as enhancers, transcriptional control elements, control sequences, etc. Any suitable spatially restricted promoter may be used and the choice of suitable promoter (e.g. a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flics, worms, mammals, mice, etc. Thus, a spatially restricted promoter can be used to regulate the expression of a nucleic acid encoding a site-specific modifying enzyme in a wide variety of different tissues and cell types, depending on the organism. Some spatially restricted promoters are also temporally restricted such that the promoter is in the “ON” state or “OFF” state during specific stages of embryonic development or during specific stages of a biological process (e.g. hair follicle cycle in mice). For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g. EMBL EISEN02, X51956); an aromatic amino acid decarboxylase (AADC) promoter; a neurofilament promoter (see, e.g. GenBank EIUMNFL, L04147); a synapsin promoter (see, e.g. GenBank EIUMSYNIB, M55301); a thy-1 promoter (see, e.g. Chen et al. (1987) Cell 51:7-19; and Llewellyn, et al. (2010) Nat. Med. 16 (10): 1161-1166); a serotonin receptor promoter (see, e.g. GenBank S62283); a tyrosine hydroxylase promoter (TH) (see, e.g. Oh et al. (2009) Gene Ther. 16:437; Sasaoka et al. (1992) Mol. Brain Res. 16:274; Boundy et al. (1998) J. Neurosci. 18:9989; and Kaneda et al. (1991) Neuron 6:583-594); a GnREI promoter (see, e.g. Radovick et al. (1991) Proc. Natl. Acad. Sci. USA 88:3402-3406); an L7 promoter (sec. e.g. Oberdick et al. (1990) Science 248:223-226); a DNMT promoter (see, e.g. Bartge et al. (1988) Proc. Natl. Acad. Sci. USA 85:3648-3652); an enkephalin promoter (see, e.g. Comb et al. (1988) EMBO J. 17:3793-3805); a myelin basic protein (MBP) promoter; a Ca2+-calmodulin-dependent protein kinase 11-alpha (CamKIM) promoter (see, e.g. Mayford et al. (1996) Proc. Natl. Acad. Sci. USA 93:13250; and Casanova et al. (2001) Genesis 31:37); a CMV enhancer/platelet-derived growth factor-p promoter (see, e.g. Liu et al. (2004) Gene Therapy 11:52-60); and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g. guide RNA) or a coding sequence (e.g. M-SmallCas9 polypeptide or variant thereof) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “chimeric” as used herein as applied to a nucleic acid or polypeptide refers to one entity that is composed of structures derived from different sources. For example, where “chimeric” is used in the context of a chimeric polypeptide (e.g. a chimeric M-SmallCas9 protein), the chimeric polypeptide includes amino acid sequences that are derived from different polypeptides. A chimeric polypeptide may include either modified or naturally-occurring polypeptide sequences (e.g. a first amino acid sequence from a modified or unmodified M-SmallCas9 protein; and a second amino acid sequence other than the M-SmallCas9 protein). Similarly. “chimeric” in the context of a polynucleotide encoding a chimeric polypeptide includes nucleotide sequences derived from different coding regions.

The term “chimeric polypeptide” refers to a polypeptide which is not naturally occurring. e.g. is made by the artificial combination (e.g., “fusion”) of two or more otherwise separated segments of amino sequence through human intervention. A polypeptide that includes a chimeric amino acid sequence is a chimeric polypeptide. Some chimeric polypeptides can be referred to as “fusion variants.”

“Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). In addition, or alternatively. DNA sequences encoding RNA (e.g. guide RNA) that is not translated may also be considered recombinant. Thus, e.g. the term “recombinant” nucleic acid refers to one which is not naturally occurring. e.g. is made by the artificial combination of two otherwise separated segments of sequence through human intervention. This artificial combination is often accomplished by either chemical synthesis means, or by the artificial manipulation of isolated segments of nucleic acids. e.g. by genetic engineering techniques. Such is generally done to replace a codon with a codon encoding the same amino acid, a conservative amino acid, or a non-conservative amino acid. In addition or alternatively, it is performed to join together nuclei acid segments of desired functions to generate a desired combination of functions. This artificial combination is often accomplished by either chemical synthesis means, or by the artificial manipulation of isolated segments of nucleic acids. e.g. by genetic engineering techniques. When a recombinant polynucleotide encodes a polypeptide, the sequence of the encoded polypeptide can be naturally occurring (“wild type”) or can be a variant (e.g. a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g. a variant, a mutant, etc.). Thus, a “recombinant” polypeptide is the result of human intervention, but may be a naturally occurring amino acid sequence. The term “non-naturally occurring” includes molecules that are markedly different from their naturally occurring counterparts, including chemically modified or mutated molecules.

A “vector” or “expression vector” is a replicon, such as plasmid, phage, virus, or cosmid, to which another DNA segment, e.g., an “insert”, may be attached so as to bring about the replication of the attached segment in a cell.

An “expression cassette” includes a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector.” or “DNA construct” are used interchangeably herein to refer to a DNA molecule comprising a vector and at least one insert. Recombinant expression vectors are generally generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non contiguous.

A cell has been “genetically modified” or “transformed” or “transfected” by exogenous DNA. e.g. a recombinant expression vector, when such DNA has been introduced inside the cell. The presence of the exogenous DNA results in permanent or transient genetic change. The transforming DNA may or may not be integrated (covalently linked) into the genome of the cell.

In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that include a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include but are not limited to, e.g. viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al, Adv Drug Deliv Rev. 2012 Sep. 13. pp: S0169-409X (12) 00283-9. doi: 10.1016/j.addr.2012.09.023), and the like.

A “host cell,” as used herein, denotes an in vivo or in vitro eukaryotic cell, a prokaryotic cell (e.g. bacterial or archacal cell), or a cell from a multicellular organism (e.g. a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g. an expression vector. For example, a bacterial host cell is a genetically modified bacterial host cell by virtue of introduction into a suitable bacterial host cell of an exogenous nucleic acid (e.g. a plasmid or recombinant expression vector) and a eukaryotic host cell is a genetically modified eukaryotic host cell (e.g. a mammalian germ cell), by virtue of introduction into a suitable eukaryotic host cell of an exogenous nucleic acid.

“Nuclease” and “endonuclease” are used interchangeably herein to mean an enzyme which possesses endonucleolytic catalytic activity for polynucleotide cleavage. By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

The “guide sequence” or “DNA-targeting segment” or “DNA-targeting sequence” or “spacer” includes a nucleotide sequence that is complementary to a specific sequence within a target DNA (the complementary strand of the target DNA) designated the “protospacer-like” sequence herein. The protein-binding segment (or “protein-binding sequence”) interacts with a site-specific modifying enzyme. When the site-specific modifying enzyme is a M-SmallCas9 or M-SmallCas9-related polypeptide (described in more detail below), site-specific cleavage of the target DNA occurs at locations determined by both (i) base pairing complementarity between the guide RNA and the target DNA; and (ii) a short motif (referred to as the protospacer adjacent motif (PAM)) in the target DNA. The protein-binding segment of a guide RNA includes, in part, two complementary stretches of nucleotides that hybridize to one another to form a double stranded RNA duplex (dsRNA duplex). In some embodiments, a nucleic acid (e.g. a guide RNA, a nucleic acid comprising a nucleotide sequence encoding a guide RNA; a nucleic acid encoding a site-specific modifying enzyme; etc.) includes a modification or sequence that provides for an additional desirable feature (e.g. modified or regulated stability; subcellular targeting; tracking, e.g. a fluorescent label; a binding site for a protein or protein complex; etc.). Non-limiting examples include: a 5′ cap (e.g. a 7-methylguanylate cap (m7G)); a 3′ polyadenylated tail (e.g., a 3′ poly(A) tail); a riboswitch sequence (e.g. to allow for regulated stability and/or regulated accessibility by proteins and/or protein complexes); a stability control sequence; a sequence that forms a dsRNA duplex (e.g., a hairpin)); a modification or sequence that targets the RNA to a subcellular location (e.g. nucleus, mitochondria, chloroplasts, and the like); a modification or sequence that provides for tracking (e.g. direct conjugation to a fluorescent molecule, conjugation to a moiety that facilitates fluorescent detection, a sequence that allows for fluorescent detection, etc.); a modification or sequence that provides a binding site for proteins (e.g. proteins that act on DNA, including transcriptional activators, transcriptional repressors, DNA methyltransferases, DNA demethylases, histone acetyltransferases, histone deacetylases, and the like); and combinations thereof.

In some embodiments, a guide RNA includes an additional segment at either the 5′ or 3′ end that provides for any of the features described above. For example, a suitable third segment can include a 5′ cap (e.g. a 7-methylguanylate cap (m7G)); a 3′ polyadenylated tail (e.g., a 3′ poly(A) tail); a riboswitch sequence (e.g. to allow for regulated stability and/or regulated accessibility by proteins and protein complexes); a stability control sequence; a sequence that forms a dsRNA duplex (e.g., a hairpin)); a sequence that targets the RNA to a subcellular location (e.g. nucleus, mitochondria, chloroplasts, and the like); a modification or sequence that provides for tracking (e.g. direct conjugation to a fluorescent molecule, conjugation to a moiety that facilitates fluorescent detection, a sequence that allows for fluorescent detection, etc.); a modification or sequence that provides a binding site for proteins (e.g. proteins that act on DNA. including transcriptional activators, transcriptional repressors, DNA methyltransferases, DNA demethylases, histone acetyltransferases, histone deacetylases, and the like); and combinations thereof.

A guide RNA and a site-specific modifying enzyme such as a M-SmallCas9 polypeptide or variant thereof may form a ribonucleoprotein complex (e.g., bind via non-covalent interactions). The guide RNA provides target specificity to the complex by comprising a nucleotide sequence that is complementary to a sequence of a target DNA. The site-specific modifying enzyme of the complex provides the endonuclease activity. In other words, the site-specific modifying enzyme is guided to a target DNA sequence (e.g. a target sequence in a chromosomal nucleic acid; a target sequence in an extrachromosomal nucleic acid. e.g. an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA. RNA aptamers are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the RNA molecule of which they are part. RNA aptamers generally include a sequence that folds into a particular structure (e.g. a hairpin), which specifically binds a particular drug (e.g. a small molecule). Binding of the drug causes a structural change in the folding of the RNA, which changes a feature of the nucleic acid of which the aptamer is a part. As non-limiting examples: (i) an activator-RNA with an aptamer may not be able to bind to the cognate targeter RNA unless the aptamer is bound by the appropriate drug; (ii) a targeter-RNA with an aptamer may not be able to bind to the cognate activator-RNA unless the aptamer is bound by the appropriate drug; and (iii) a targeter-RNA and an activator-RNA, each comprising a different aptamer that binds a different drug, may not be able to bind to each other unless both drugs are present. As illustrated by these examples, a two-molecule guide RNA can be designed to be inducible.

Examples of aptamers and riboswitches can be found. for example, in: Nakamura et al., Genes Cells. 2012 May; 17 (5): 344-64; Vavalle et al., Future Cardiol. 2012 May; 8 (3): 371-82; Citartan et al., Biosens Bioelectron. 2012 April 15; 34 (1): 1-11; and Liberman et al., Wiley Interdiscip Rev RNA. 2012 May-June; 3 (3): 369-84; all of which are herein incorporated by reference in their entireties.

The choice of method of genetic modification is generally dependent on the type of cell being transformed and the circumstances under which the transformation is taking place (e.g. in vitro, ex vivo, or in vivo). A general discussion of these methods can be found in Ausubel, et al., Short Protocols in Molecular Biology. 3rd ed., Wiley & Sons, 1995.

Examples of aptamers and riboswitches can be found, for example, in: Nakamura et al. Genes Cells. 2012 May; 17 (5): 344-64; Vavalle et al. Future Cardiol. 2012 May; 8 (3): 371-82; Citartan et al., Biosens Bioelectron. 2012 Apr. 15; 34 (1): 1-11; and Liberman et al. Wiley Interdiscip Rev RNA. 2012 May-June; 3 (3): 369-84; all of which are herein incorporated by reference in their entirety.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom. e.g., arresting its development; or (c) relieving the disease, e.g., causing regression of the disease. The therapeutic agent may be administered before, during or after the onset of disease or injury. The treatment of ongoing disease, where the treatment stabilizes or reduces the undesirable clinical symptoms of the subject, is of particular interest. Such treatment is desirably performed prior to complete loss of function in the affected tissues. The therapy will desirably be administered during the symptomatic stage of the disease, and in some cases after the symptomatic stage of the disease.

The terms “individual,” “subject.” “host.” and “patient.” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (1. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

The phrase “consisting essentially of is meant herein to exclude anything that is not the specified active component or components of a system, or that is not the specified active portion or portions of a molecule.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

The following list of numbered embodiments are disclosed:
Embodiment 1. A nucleic acid delivery system, comprising:
   a delivery vector, the delivery vector comprising:
      a nucleic acid comprising a polynucleotide encoding:
         a chimeric receptor, the chimeric receptor comprising:
         a cytoplasmic domain;
         a transmembrane domain; and
         an extracellular domain; and
      a nucleic acid inhibitor that decreases expression of a protein that targets the cytoplasmic domain for degradation.
Embodiment 2. The nucleic acid delivery system of embodiment 1, further comprising a targeting agent, wherein the targeting agent directs the delivery vector to a cell; and wherein the extracellular domain of the chimeric receptor is other than any wild-type extracellular domain found in any wild-type protein comprising the same cytoplasmic portion as the chimeric receptor.
Embodiment 3. The nucleic acid delivery system of embodiments 1 and 2, wherein the targeting agent also acts as an activating agent for the cell.
Embodiment 4. The nucleic acid delivery system of any one of the preceding embodiments, wherein the cell is a leukocyte.
Embodiment 5. The nucleic acid delivery system of any one of the preceding embodiments, wherein the leukocyte is a monocyte, for example a macrophage or dendritic cell.
Embodiment 6. The nucleic acid delivery system of any one of the preceding embodiments, wherein the cell is in vivo.
Embodiment 7. The nucleic acid delivery system of any one of the preceding embodiments, wherein the cell in the monocyte lineage is expressing the chimeric receptor.
Embodiment 8. The nucleic acid delivery system of any one of the preceding embodiments, wherein the nucleic acid is an RNA.
Embodiment 9. The nucleic acid delivery system of any one of the preceding embodiments, wherein activation of the cytoplasmic portion of the chimeric receptor polarizes the macrophage or dendritic cell to an M1 macrophage.
Embodiment 10. The nucleic acid delivery system of any one of the preceding embodiments, wherein activation of the cytoplasmic portion of the chimeric receptor polarizes the macrophage or dendritic cell to an M2 macrophage.
Embodiment 11. The nucleic acid delivery system of any one of the preceding embodiments, wherein the extracellular domain is an antibody.
Embodiment 12. The nucleic acid delivery system of any one of the preceding embodiments, wherein the protein is RFN216 or Rab7b.
Embodiment 13. The nucleic acid delivery system of any one of the preceding embodiments, wherein the nucleic acid inhibitor is an shRNA that is not integrated into the genome.
Embodiment 14. The nucleic acid delivery system of any one of the preceding embodiments, wherein the binding of a ligand to the extracellular domain of the chimeric receptor initiates downstream signaling in the cytoplasmic portion.
Embodiment 15. The nucleic acid delivery system of any one of the preceding embodiments, wherein the cytoplasmic portion of the chimeric receptor comprises a cytoplasmic domain from a toll-like receptor, myeloid differentiation primary response protein (MYD88), toll-like receptor 3 (TLR3), toll-like receptor 4 (TLR4), toll-like receptor 7 (TLR7), toll-like receptor 8 (TLR8), toll-like receptor 9 (TLR9), myelin and lymphocyte protein (MAL), interleukin-1 receptor associated kinase 1 (IRAK1), low affinity immunoglobulin gamma Fc region receptor III-A (FCGR3A), low affinity immunoglobulin gamma Fc region receptor II-a (FCGR2A), and high affinity immunoglobulin epsilon receptor subunit gamma (FCER1G).
Embodiment 16. The nucleic acid delivery system of any one of the preceding embodiments, wherein the ligand is selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), B7-H3, PSCA, PSMA, CD19, CD20, CD30, CD33, CD123, CD133, CD138, and CD171.
Embodiment 17. The nucleic acid delivery system of any one of the preceding embodiments, wherein the extracellular domain of the chimeric receptor is an antibody or fragment thereof specific for a ligand selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.
Embodiment 18. The nucleic acid delivery system of any one of the preceding embodiments, wherein the antibody or fragment thereof is a single-chain variable fragment (scFv) or a single-domain antibody (sdAb).
Embodiment 19. The nucleic acid delivery system of any one of the preceding embodiments, wherein the chimeric receptor further comprises a hinge between the transmembrane domain and the extracellular domain.
Embodiment 20. The nucleic acid delivery system of any one of the preceding embodiments, wherein the targeting agent is protein based.
Embodiment 21. The nucleic acid delivery system of any one of the preceding embodiments, wherein the targeting agent is lipid based.
Embodiment 22. The nucleic acid delivery system of any one of the preceding embodiments, wherein activation of the cytoplasmic portion of the chimeric receptor polarizes the cell in the monocyte lineage.
Embodiment 23. The nucleic acid delivery system of any one of the preceding embodiments, wherein the nucleic acid comprises a promoter operably linked to the polynucleotide.
Embodiment 24. The nucleic acid delivery system of any one of the preceding embodiments, wherein the promoter is selected from the group consisting of CD68 promoter (for example CD68/150(r)), EF1α, CMV, lysM, csflr, CD11c, SRA, and CDF11b.
Embodiment 25. The nucleic acid delivery system of any one of the preceding embodiments, wherein the delivery vector is a liposome.
Embodiment 26. The nucleic acid delivery system of any one of the preceding embodiments, wherein the delivery vector is a lipid nanoparticle.
Embodiment 27. The nucleic acid delivery system of any one of the preceding embodiments, wherein the targeting agent is a monocyte ligand.
Embodiment 28. The nucleic acid delivery system of any one of the preceding embodiments, wherein the monocyte ligand is selected from the group consisting of toll-like receptor, GM-CSF, CD14, CD16, CD64, CD115, CD192, CX2CR1, CD226, CD284, CD155, or any combination thereof comprises anyone of CD3, CD4, CD8, CD23, or CD28.
Embodiment 29. A cell expressing the chimeric receptor and the nucleic acid inhibitor of embodiment 1, wherein the cell is a leukocyte.
Embodiment 30. The cell of embodiment 29, wherein the leukocyte is monocyte, for example a macrophage or dendritic cell.
Embodiment 31. The cell of any one of embodiments 29 to 30, wherein activation of the cytoplasmic portion of the chimeric receptor polarizes a macrophage to a M1 macrophage.
Embodiment 32. The leukocyte of any one of embodiments 29 to 31, wherein activation of the cytoplasmic portion of the chimeric receptor polarizes a macrophage to a M2 macrophage.
Embodiment 33. A delivery system for a chimeric antigen receptor, the system comprising:
   a shell encasing mRNA encoding a chimeric receptor;
      the chimeric receptor comprising:
         a cytoplasmic domain;
         a transmembrane domain; and
         an extracellular domain; and
         wherein a wild-type protein comprising the cytoplasmic portion does not comprise the extracellular domain.
Embodiment 34. A method of providing a nucleic acid to a cell, the method comprising:
   providing the delivery vector of embodiment 1 or the delivery system of embodiment 33 to a patient; and
   delivering the nucleic acid into the cell.
Embodiment 35. The method of embodiment 34, further comprising a targeting agent wherein the targeting agent directs the delivery vector to the cell.
Embodiment 36. The method of embodiments 34 and 35, wherein the targeting agent also acts as an activating agent for the cell.
Embodiment 37. The method of any one of embodiments 34 to 36, wherein the cell is a leukocyte.
Embodiment 38. The method of any one of embodiments 34 to 37, wherein the leukocyte is a monocyte, for example macrophage or dendritic cell.
Embodiment 39. The method of any one of embodiments 34 to 38, wherein the cell is in vivo.
Embodiment 40. The method of any one of embodiments 34 to 39, wherein the cell in the monocyte lineage is expressing the chimeric receptor.
Embodiment 41. The method of any one of embodiments 34 to 40, wherein the nucleic acid is an RNA.
Embodiment 42. The method of any one of embodiments 34 to 41, further comprising: providing a ligand to the extracellular domain of the chimeric receptor.
Embodiment 43. The method of any one of embodiments 34 to 42, further comprising: binding the ligand to the extracellular domain of the chimeric receptor.
Embodiment 44. The method of any one of embodiments 34 to 43, wherein the binding of the ligand to the extracellular domain of the chimeric receptor initiates downstream signaling in the cytoplasmic portion.
Embodiment 45. The method of any one of embodiments 34 to 44, wherein activation of the cytoplasmic portion polarizes the macrophage or dendritic cell.
Embodiment 46. The method of any one of embodiments 34 to 45, wherein activation of the cytoplasmic portion of the chimeric receptor polarizes a macrophage or dendritic cell to an M1 macrophage.
Embodiment 47. The method of any one of embodiments 34 to 46, wherein activation of the cytoplasmic portion of the chimeric receptor polarizes a macrophage or dendritic cell to an M2 macrophage.
Embodiment 48. The method of any one of embodiments 34 to 47, wherein the extracellular domain is an antibody.
Embodiment 49. The method of any one of embodiments 34 to 48, wherein the protein is RFN216 or Rab7b.
Embodiment 50. The method of any one of embodiments 34 to 49, wherein the nucleic acid inhibitor is an shRNA that is not integrated into the genome.
Embodiment 51. The method of any one of embodiments 34 to 50, wherein the cytoplasmic portion of the chimeric receptor comprises a cytoplasmic domain from a toll-like receptor, myeloid differentiation primary response protein (MYD88), toll-like receptor 3 (TLR3), toll-like receptor 4 (TLR4), toll-like receptor 7 (TLR7), toll-like receptor 8 (TLR8), toll-like receptor 9 (TLR9), myelin and lymphocyte protein (MAL), interleukin-1 receptor associated kinase 1 (IRAK1), low affinity immunoglobulin gamma Fc region receptor III-A (FCGR3A), low affinity immunoglobulin gamma Fc region receptor II-a (FCGR2A), and high affinity immunoglobulin epsilon receptor subunit gamma (FCER1G).
Embodiment 52. The method of any one of embodiments 34 to 51, wherein the ligand is selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.
Embodiment 53. The method of any one of embodiments 34 to 52, wherein the extracellular domain of the chimeric receptor is an antibody or fragment thereof specific for a ligand selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.
Embodiment 54. The method of any one of embodiments 34 to 53, wherein the antibody or fragment thereof is a single-chain variable fragment (scFv) or a single-domain antibody (sdAb).
Embodiment 55. The method of any one of embodiments 34 to 54, wherein the chimeric receptor further comprises a hinge between the transmembrane domain and the extracellular domain.
Embodiment 56. The method of any one of embodiments 34 to 55, wherein the targeting agent is protein based.
Embodiment 57. The method of any one of embodiments 34 to 56, wherein the targeting agent is lipid based.
Embodiment 58. The method of any one of embodiments 34 to 57, wherein activation of the cytoplasmic portion of the chimeric receptor polarizes the cell in the monocyte lineage.
Embodiment 59. The method of any one of embodiments 34 to 58, wherein the nucleic acid comprises a promoter operably linked to the polynucleotide.
Embodiment 60. The method of any one of embodiments 34 to 59, wherein the promoter is selected from the group consisting of CD68 promoter (for example CD68/150(r)), EF1α, CMV, lysM, csflr, CD11c, SRA, and CDF11b.
Embodiment 61. The method of any one of embodiments 34 to 60, wherein the delivery vector is a liposome.
Embodiment 62. The method of any one of embodiments 34 to 61, wherein the delivery vector is a lipid nanoparticle.
Embodiment 63. The method of any one of embodiments 34 to 62, wherein the delivery vector has an outer shell comprising a lipid hydrophobic tail and a hydrophilic head.
Embodiment 64. The method of any one of embodiments 34 to 63, wherein the targeting agent is a monocyte ligand.
Embodiment 65. The method of any one of embodiments 34 to 64, wherein the monocyte ligand is selected from the group consisting of toll-like receptor, GM-CSF, CD14, CD16, CD64, CD115, CD192, CX2CR1, CD226, CD284, CD155, or any combination thereof comprises anyone of CD3, CD4, CD8, CD23, or CD28.
Embodiment 66. A method of providing a nucleic acid to a cell, the method comprising: providing the delivery system of embodiment 33 to a patient; and delivering the nucleic acid into the cell.
Embodiment 67. The method of embodiment 66, further comprising a targeting agent wherein the targeting agent directs the delivery vector to the cell.
Embodiment 68. The method of embodiments 66 and 67, wherein the targeting agent also acts as an activating agent for the cell.
Embodiment 69. The method of any one of embodiments 66 to 68, wherein the cell is a leukocyte.
Embodiment 70. The method of any one of embodiments 66 to 69, wherein the leukocyte is a monocyte, for example a macrophage or dendritic cell.
Embodiment 71. The method of any one of embodiments 66 to 70, wherein the cell is in vivo.
Embodiment 72. The method of any one of embodiments 66 to 71, wherein the cell in the monocyte lineage is expressing the chimeric receptor.
Embodiment 73. The method of any one of embodiments 66 to 72, further comprising: providing a ligand to the extracellular domain of the chimeric receptor.
Embodiment 74. The method of any one of embodiments 66 to 73, further comprising: binding the ligand to the extracellular domain of the chimeric receptor.
Embodiment 75. The method of any one of embodiments 66 to 74, wherein the binding of the ligand to the extracellular domain of the chimeric receptor initiates downstream signaling in the cytoplasmic portion.
Embodiment 76. The method of any one of embodiments 66 to 75, wherein activation of the cytoplasmic portion polarizes the macrophage or dendritic cell.
Embodiment 77. The method of any one of embodiments 66 to 76, wherein activation of the cytoplasmic portion of the chimeric receptor polarizes a macrophage to an M1 macrophage.
Embodiment 78. The method of any one of embodiments 66 to 77, wherein activation of the cytoplasmic portion of the chimeric receptor polarizes a macrophage to an M2 macrophage.
Embodiment 79. The method of any one of embodiments 66 to 78, wherein the cytoplasmic portion of the chimeric receptor comprises a cytoplasmic domain from a toll-like receptor, myeloid differentiation primary response protein (MYD88), toll-like receptor 3 (TLR3), toll-like receptor 4 (TLR4), toll-like receptor 7 (TLR7), toll-like receptor 8 (TLR8), toll-like receptor 9 (TLR9), myelin and lymphocyte protein (MAL), interleukin-1 receptor associated kinase 1 (IRAK1), low affinity immunoglobulin gamma Fc region receptor III-A (FCGR3A), low affinity immunoglobulin gamma Fc region receptor II-a (FCGR2A), and high affinity immunoglobulin epsilon receptor subunit gamma (FCER1G).
Embodiment 80. The method of any one of embodiments 66 to 79, wherein the ligand is selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.
Embodiment 81. The method of any one of embodiments 66 to 80, wherein the extracellular domain of the chimeric receptor is an antibody or fragment thereof specific for a ligand selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.
Embodiment 82. The method of any one of embodiments 66 to 81, wherein the extracellular domain is an antibody.
Embodiment 83. The method of any one of embodiments 66 to 82, wherein the antibody or fragment thereof is a single-chain variable fragment (scFv) or a single-domain antibody (sdAb).
Embodiment 84. The method of any one of embodiments 66 to 83, wherein the targeting agent is protein based.
Embodiment 85. The method of any one of embodiments 66 to 84, wherein the targeting agent is lipid based.
Embodiment 86. The method of any one of embodiments 66 to 85, wherein the chimeric receptor further comprises a hinge region between the transmembrane domain and the linker.
Embodiment 87. The method of any one of embodiments 66 to 86, wherein the nucleic acid comprises a promoter operably linked to the polynucleotide.
Embodiment 88. The method of any one of embodiments 66 to 87, wherein the promoter is selected from the group consisting of CD68 promoter (for example CD68/150(r)), EF1α, CMV, lysM, csflr, CD11c, SRA, and CDF11b.
Embodiment 89. The method of any one of embodiments 66 to 88, wherein the shell is a liposome.
Embodiment 90. The method of any one of embodiments 66 to 89, wherein the shell is a lipid nanoparticle.
Embodiment 91. The method of any one of embodiments 66 to 90, wherein the targeting agent is a monocyte ligand.
Embodiment 92. The method of any one of embodiments 66 to 91, wherein the monocyte ligand is selected from the group consisting of toll-like receptor, GM-CSF, CD14, CD16, CD64, CD115, CD192, CX2CR1, CD226, CD284, CD155, or any combination thereof comprises anyone of CD3, CD4, CD8, CD23, or CD28.
Embodiment 93. The method of any one of embodiments 66 to 92, wherein activation of the cytoplasmic portion of the chimeric receptor polarizes the cell in the monocyte lineage.
Embodiment 94. A method of producing a polarized macrophage in a subject, the method comprising:
   administering to the subject a nanoparticle, the nanoparticle comprising a nucleic acid encoding a chimeric antigen receptor;
      wherein the chimeric receptor comprises:
         a cytoplasmic domain;
         a transmembrane domain; and
         an extracellular domain;
         wherein the extracellular domain of the chimeric receptor is other than any wild-type extracellular domain found in any wild-type protein comprising the same cytoplasmic portion as the chimeric receptor;
         wherein the extracellular domain specifically binds a target; and
         wherein binding of the target by the extracellular domain activates the cytoplasmic domain to polarize a macrophage,
   wherein the nanoparticle is taken up by a monocyte or the macrophage in the subject;
   wherein the nucleic acid is expressed in the monocyte or macrophage to produce the chimeric antigen receptor; and
   wherein the extracellular domain binds specifically to its target in the subject and thereby signals the monocyte or macrophage to become a polarized macrophage.
Embodiment 95. The method according to embodiment 94, wherein the nanoparticle further comprises a targeting agent, wherein the targeting agent directs uptake of the nanoparticle by the monocyte or macrophage.
Embodiment 96. The method according to embodiment 94, wherein the nanoparticle further comprises a macrophage activating agent.
Embodiment 97. The method according to embodiment 97, wherein the activating agent and the targeting agent are the same.
Embodiment 98. The method according to embodiment 95, wherein the targeting agent is mannose.
Embodiment 99. The method according to embodiment 94, wherein the cytoplasmic domain comprises an antigen binding antibody fragment.
Embodiment 100. The method according to embodiment 99, wherein the cytoplasmic domain polarizes the monocyte or macrophage to a M1 phenotype macrophage upon binding of the antibody fragment to its antigen.
Embodiment 101. The method according to embodiment 99, wherein the antibody fragment is a single chain variable fragment (scFv).
Embodiment 102. The method according to embodiment 101, wherein the scFv is derived from a monoclonal antibody specific for the antigen expressed by cells of a cancer.
Embodiment 103. The method according to embodiment 102, wherein the monoclonal antibody is a human or mouse monoclonal antibody.
Embodiment 104. The method according to embodiment 94, wherein the target is present on cells of a cancer.
Embodiment 105. The method according to embodiment 94, wherein the monocyte or macrophage are present in a tumor.
Embodiment 106. The method according to embodiment 94, wherein the monocyte or macrophage are a tumor associated macrophage.
Embodiment 107. A method of producing a polarized tumor associated macrophage (TAM) in a subject, the method comprising:
   administering to the subject a nanoparticle, the nanoparticle comprising a nucleic acid encoding a chimeric antigen receptor;
      wherein the chimeric receptor comprises:
         a cytoplasmic domain;
         a transmembrane domain; and
         an extracellular domain;
         wherein the extracellular domain of the chimeric receptor is other than any wild-type extracellular domain found in any wild-type protein comprising the same cytoplasmic portion as the chimeric receptor;
         wherein the extracellular domain specifically binds a target; and
         wherein binding of the target by the extracellular domain activates the cytoplasmic domain to polarize a TAM,
   wherein the nanoparticle is taken up by a monocyte or the TAM in the subject;
   wherein the nucleic acid is expressed in the TAM to produce the chimeric antigen receptor; and
   wherein the extracellular domain binds specifically to its target in the subject and thereby signals the monocyte or TAM to become a polarized TAM.
Embodiment 108. The method according to embodiment 107, wherein the nanoparticle further comprises a targeting agent, wherein the targeting agent directs uptake of the nanoparticle by the monocyte or TAM.
Embodiment 109. The method according to embodiment 107, wherein the nanoparticle further comprises a TAM activating agent.
Embodiment 110. The method according to embodiment 109, wherein the activating agent and the targeting agent are the same.
Embodiment 111. The method according to embodiment 108, wherein the targeting agent is mannose.
Embodiment 112. The method according to embodiment 107, wherein the cytoplasmic domain comprises an antigen binding antibody fragment.
Embodiment 113. The method according to embodiment 112, wherein the cytoplasmic domain polarizes the monocyte or TAM to a M1 phenotype TAM upon binding of the antibody fragment to its antigen.
Embodiment 114. The method according to embodiment 112, wherein the antibody fragment is a single chain variable fragment (scFv).
Embodiment 115. The method according to embodiment 114, wherein the scFv is derived from a monoclonal antibody specific for the antigen expressed by cells of a cancer.
Embodiment 116. The method according to embodiment 115, wherein the monoclonal antibody is a human or mouse monoclonal antibody.
Embodiment 117. The method according to embodiment 107, wherein the target is present on cells of a cancer.
Embodiment 118. The method according to embodiment 107, wherein the monocyte or TAM are present in a tumor. (altering tumor)
Embodiment 119. The method according to embodiment 107, wherein the monocyte or TAM are a tumor associated TAM.
Embodiment 120. A method of altering a tumor in a subject, the method comprising:
   administering to the subject a nanoparticle, the nanoparticle comprising a nucleic acid encoding a chimeric antigen receptor;
      wherein the chimeric receptor comprises:
         a cytoplasmic domain;
         a transmembrane domain; and
         an extracellular domain;
         wherein the extracellular domain of the chimeric receptor is other than any wild-type extracellular domain found in any wild-type protein comprising the same cytoplasmic portion as the chimeric receptor;
         wherein the extracellular domain specifically binds a target; and
         wherein binding of the target by the extracellular domain activates the cytoplasmic domain to polarize a macrophage,
   wherein the nanoparticle is taken up by a monocyte or a macrophage in the subject;
   wherein the nucleic acid is expressed in the macrophage to produce the chimeric antigen receptor;
   wherein the extracellular domain binds specifically to its target in the subject and thereby signals the monocyte or macrophage to become a polarized macrophage
   wherein the macrophages are present in the tumor; and
   wherein the macrophages are tumor associated macrophages.
Embodiment 121. The method according to embodiment 120, wherein the nanoparticle further comprises a targeting agent, wherein the targeting agent directs uptake of the nanoparticle by the monocyte or the macrophage.
Embodiment 122. The method according to embodiment 120, wherein the nanoparticle further comprises a macrophage activating agent.
Embodiment 123. The method according to embodiment 122, wherein the activating agent and the targeting agent are the same.
Embodiment 124. The method according to embodiment 121, wherein the targeting agent is mannose.
Embodiment 125. The method according to embodiment 120, wherein the cytoplasmic domain comprises an antigen binding antibody fragment.
Embodiment 126. The method according to embodiment 125, wherein the cytoplasmic domain polarizes the monocyte or macrophage to a M1 phenotype macrophage upon binding of the antibody fragment to its antigen.
Embodiment 127. The method according to embodiment 125, wherein the antibody fragment is a single chain variable fragment (scFv).
Embodiment 128. The method according to embodiment 127, wherein the scFv is derived from a monoclonal antibody specific for the antigen expressed by cells of a cancer. (method treating cancer)
Embodiment 129. The method according to embodiment 128, wherein the monoclonal antibody is a human or mouse monoclonal antibody.
Embodiment 130. A method of treating a cancer in a subject, the method comprising:
   administering to the subject a nanoparticle, the nanoparticle comprising a nucleic acid encoding a chimeric antigen receptor;
      wherein the chimeric receptor comprises:
         a cytoplasmic domain;
         a transmembrane domain; and
         an extracellular domain;
         wherein the extracellular domain of the chimeric receptor is other than any wild-type extracellular domain found in any wild-type protein comprising the same cytoplasmic portion as the chimeric receptor;
         wherein the extracellular domain specifically binds a target; and
         wherein binding of the target by the extracellular domain activates the cytoplasmic domain to polarize a macrophage,
   wherein the nanoparticle is taken up by a monocyte or a macrophage in the subject;
   wherein the nucleic acid is expressed in the monocyte or the macrophage to produce the chimeric antigen receptor; and
   wherein the extracellular domain binds specifically to the cancer cells in the subject and thereby signals the monocyte or macrophage to become a polarized macrophage;
   wherein the cytoplasmic domain comprises an antigen binding antibody fragment;
   wherein the cytoplasmic domain polarizes the monocyte or the macrophage to an M1 phenotype macrophage upon binding of the antibody fragment to its antigen;
   wherein the antibody fragment is a single chain variable fragment (scFV);
   wherein the scFv is derived from a monoclonal antibody specific for the antigen expressed by cells of a cancer; and
   wherein the target is present on the cancer cells.
Embodiment 131. The method according to embodiment 130, wherein the nanoparticle further comprises a targeting agent, wherein the targeting agent directs uptake of the nanoparticle by the monocyte or macrophage.
Embodiment 132. The method according to embodiment 130, wherein the nanoparticle further comprises a macrophage activating agent.
Embodiment 133. The method according to embodiment 132, wherein the activating agent and the targeting agent are the same.
Embodiment 134. The method according to embodiment 131, wherein the targeting agent is mannose.
Embodiment 135. The method according to embodiment 130, wherein the monoclonal antibody is a human or mouse monoclonal antibody.
Embodiment 136. The method according to embodiment 130, wherein the monocyte or macrophage are present in a tumor.
Embodiment 137. The method according to embodiment 130, wherein the monocyte or macrophage are a tumor associated macrophage.

The embodiments are described in additional detail in the following illustrative examples. Although the examples may represent only selected embodiments, it should be understood that the following examples are illustrative and not limiting.

### EXAMPLES

### Example 1: Isolation of scFv Fragments for Specific Ligands

cDNA was purified from a monoclonal antibody hybridoma cell (CB1) expressing an antibody specific to human TK1. The isolated cDNA was used to amplify the heavy and light chains of the CB1 variable region via polymerase chain reaction (PCR) Sequences from the heavy and light chain were confirmed using NCBI Blast. CB1 heavy and light chains were fused together via site overlap extension (SOE) PCR to form a single-chain fragment variable (scFv) using a G4S linker. The G4S linker was codon optimized for yeast and humans using the Codon Optimization tool provided by IDT (https://www.idtdna.com/CodonOpt) in order to maximize protein expression. The CB1 scFv was cut out using restriction enzymes and inserted into a pMP71 CAR vector.

TK-1 and HPRT-specific human scFv fragments were isolated from a yeast antibody library. TK-1 and HPRT proteins were isolated. His-tagged, and purified. TK-1 and HPRT protein were labeled with an anti-His biotinylated antibody and added to the library to select for TK-1 and HPRT-specific antibody clones. TK-1 and HPRT antibody clones were alternately stained with streptavidin or anti-biotin microbeads and enriched using a magnetic column. Two additional rounds of sorting and selection were performed to isolate TK-1 and HPRT specific antibodies. For the final selection, possible TK-1 and HPRT antibody clones and their respective proteins were sorted by fluorescence-activated cell sorting (FACS) by alternately labeling with fluorescently-conjugated anti-HA or anti-c-myc antibodies to isolate TK-1 and HPRT specific antibodies. High affinity clones were selected for chimeric receptor construction. Other human antibodies or humanized antibodies from other animals could be selected or altered to be TK-1 or HPRT specific by using phage display or other recombination methods.

Selected scFv clones were then combined with human IgG1 constant domains to create an antibody for use in applications such as Western blot or ELISA in order to confirm the binding specificity of the scFv. The antibody construct was inserted into the pPNL9 yeast secretion vector and YVH10 yeast were transformed with the construct and induced to produce the antibody. Other expression systems such as E. coli or mammalian systems could also be used to secrete antibodies.

### Isolation and Characterization of Protein-Specific Antibody Fragments.

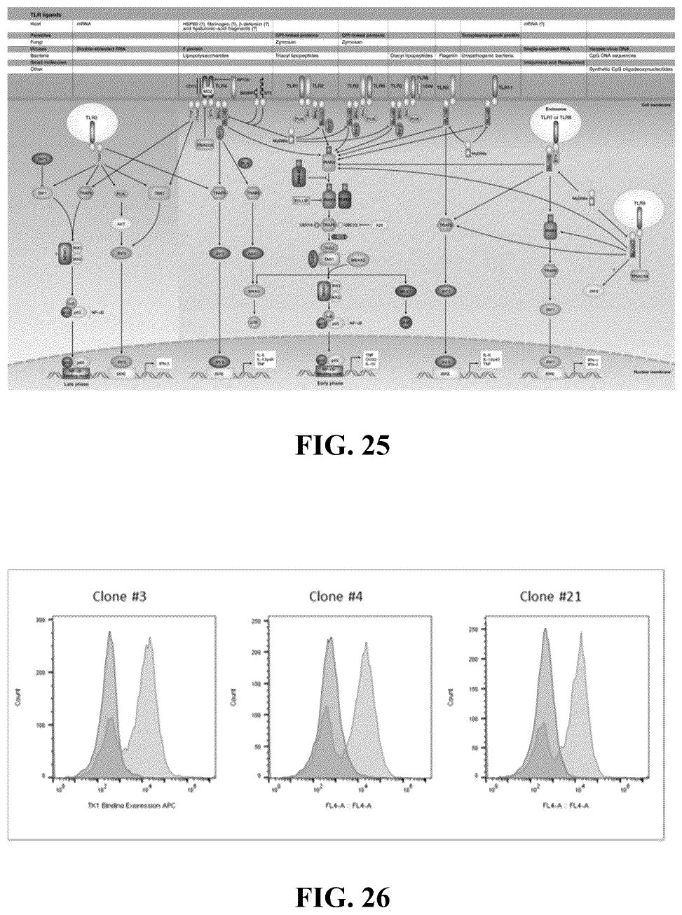

Referring to FIG. 26, 105 yeast were incubated with 2.5 ug of protein of interest labeled with the fluorescent tag APC. The higher left (red) peak indicates yeast population that was not binding to the protein of interest (negative control). The lower left (blue) peak on the left illustrates yeast not expressing their surface protein while the high (blue) peak on the right indicates binding of the expressed antibody fragment to the protein of interest.

Structural Consensus among Antibodies Defines the Antigen 5 Binding Site. PLOS Comput Biol 8 (2): c1002388. doi: 10.1371/journal.pcbi. 1002388. Kunik V. Ashkenazi S. Ofran Y (2012). Paratome: An online tool for systematic identification of antigen binding regions in antibodies based on sequence or structure. Nucleic Acids Res. 2012 July; 40 (Web Server issue): W521-4. doi: 10.1093/nar/gks480. Epub 2012 Jun. 6.

### Example 2: Creation of Chimeric Receptors

### Construction of Chimeric Receptor Vectors:

The first step in the process is the design of the nucleotide sequences for synthetic chimeric receptor genes and the selection of appropriate lentiviral vectors. All the vector design are carried out in genious software version 9.1.6. The sequences are retrieved retrieved from the Uniprot and the Human Protein Reference Data base and NCBI as well.

Vectors are synthesized with a combination of recombinant DNA techniques and gene synthesis.

Sequences for the Single chain variable fragments are produced with a humanized antibody yeast display library or a phage display library. Nucleic acids encoding ScFv specific for each of TK1, HPRT, ROR1, MUC-16, EGFRVIII, Mesothelin, HER2, CEA, BCMA, GPC3, FAP, EphA2. NKG2D ligands, GD2, CD19, CD20, CD30, CD33, CD123, CD133, CD138, and CD171. All possible combinations of nucleic acids encoding chimeric receptors having at least one of each of a), b), c), d), and e), wherein a), b), c), d), and e) are:
a) an ScFv specific for TK1, HPRT, ROR1, MUC-16, EGFRVIII, Mesothelin, HER2, CEA, BCMA, GPC3, FAP, EphA2, NKG2D ligands, GD2, CD19, CD20, CD30, CD33, CD123, CD133, CD138, and CD171;
b) a GS linker or no GS linker;
c) A hinge region selected from an LRR 5 amino acid short hinge, a LRR long hinge, an IgG4 short hinge, an IgG 119 amino acid medium hinge, and IgG4 long hinge, a CD8 hinge, a CD8 hinge with cysteines converted to serines, and no hinge;
d) a transmembrane domain selected from the transmembrane domains of MYD88, TLR3, TLR4, TLR7, TLR8, TLR9, MAL, IRAK1, FCGR2A, FCGR3A, and FCER1G; and
e) a cytosolic domain selected from the cytosolic domains of MYD88, TLR3, TLR4, TLR7, TLR8, TLR9, MAL, IRAK1, FCGR2A, FCGR3A, and FCER1G

The foregoing nucleic acids encoding chimeric receptors are synthesized with a combination of recombinant DNA techniques and gene synthesis.

Macrophages are genetically modified with an integrated gene delivery method via lentiviral-mediated gene transfer to provide the nucleic acids encoding chimeric receptors. A third generation lentiviral system from addgene is used to package our lentiviral vectors. pHIV-dTomato (#21374) and pUltra-chilli (#48687) are the gene transfer plasmids. pCMV-VSV-G (#8454), pMDLg/pRRE (#12251), pRSV-Rev (#12253), pHCMV-AmphoEnv (#15799) are the packaging plasmids. A lentiviral mediated gene transfer of human lymphoytes has been standardized previously getting efficiencies up to 50% transduction. HEK293T cells are transfected with the calcium phosphate method (SIGMA CAPHOS). Around 10 μg of each packaging plasmid and 20 μg of vector encoding the chimeric receptor are used per transfection. After 48-36 hrs viral particles are harvested and sterile filtered. Viral titration are determined infecting HT1080 and U937 cells.

The analysis is performed by flow cytometry detecting a red fluorescent protein. After viral titration human monocytes are transduced using retronectin plates (Clonetech, T100B) and the spin infection method

Previous to lentiviral transduction, monocytes are isolated from whole PBMNCs by negative selection and magnetic sorting using the Monocyte Isolation Kit II, human (MACS130-091-153). After monocyte isolation cells are split in 2 nunclon 6 well plates (Thermo, 145380) seeding 1.5×106 cells in each well for each vector. One plate is immediately transduced while the second plate is used for ex-vivo differentiation of monocytes to M1 macrophages. The M1 macrophages are produced using the media M1-Macrophage Generation Medium DFX (Promocell, C-28055). After 7 days, macrophages are transduced and activated at day 9 with LPS (500X) (Affimetryx, 00-4976-03) and IFN-γ (Promokine, C-60724). The transduction efficiency is analyzed by flow cytometry. Transduced cells are separated by cell sorting using a FACS Aria cell sorter. After cell sorting transduced monocytes are ex vivo cultured for a couple of days before differentiation while differentiated macrophages can last a month.

### Example 3: Polarization of Macrophages Through Chimeric Receptors

The transduced macrophages prepared in Example 2 are separately exposed to TK1, HPRT, ROR1, MUC-16, EGFRVIII, Mesothelin, HER2, CEA, BCMA, GPC3, FAP, EphA2, NKG2D conjugated ligands, GD2, CD19, CD20, CD30, CD33, CD123, CD133, CD138, and CD171 and tested for polarization to the M1 phenotype by monitoring the secretion of IL-12 and IL-23 using a standard cytokine assay or by measuring RNA production. Macrophages bearing chimeric receptors are polarized to the M1 phenotype when exposed to the ligand specific for the particular chimeric receptor and determined by increased secretion of IL-12 and/or IL-23. Ligands other than the specific ligand for the specific chimeric receptor display no increase in IL-12 and/or IL-21.

### Example 4: Production of Monocyte-Derived Macrophages and Transduction

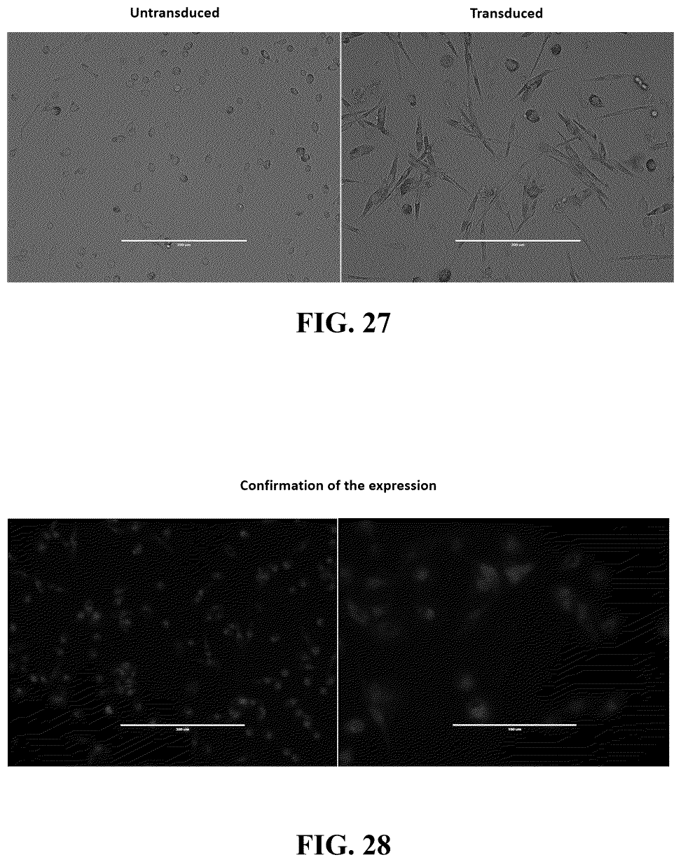

After 7 days of differentiation monocyte-derived macrophages had undergone phenotype changes. These changes where compared between transduced and non-transduced cells. As can be observed in FIG. 27, transduced cells have a more aggressive phenotype similar to M1 or classically activated macrophages. FIG. 27 shows images of Non-transduced and transduced monocyte-derived macrophages at day 8 of differentiation. No Interferon gamma and LPS was added at this point. It can be observed that the phenotype of macrophages transduced with a chimeric receptor is different from non-transduced macrophages. Transduced cells displayed a classically activated or M1-like phenotype indicating macrophage activation. The altered phenotype may be a combined effect of the transduction process and the expression of the new synthetic receptor.

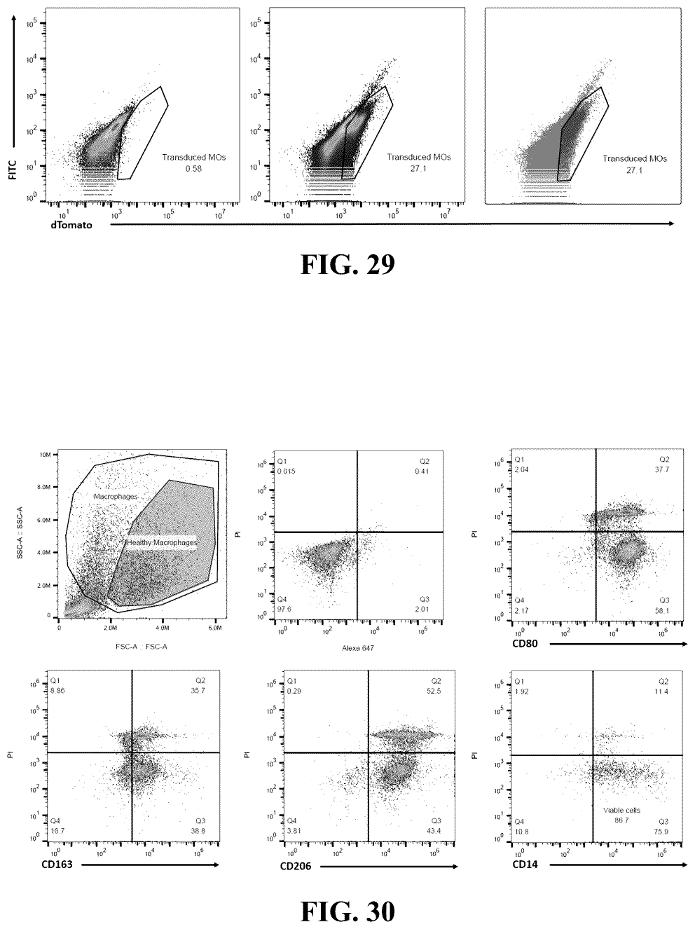

FIG. 28 provides confirmation of the insertion and expression of constructs encoding a chimeric receptor as was confirmed by the expression of dTomato 48-72 hrs. after transduction. This demonstrates the successful transduction of human monocyte-derived macrophages.

### Example 5: Transduction Efficiency

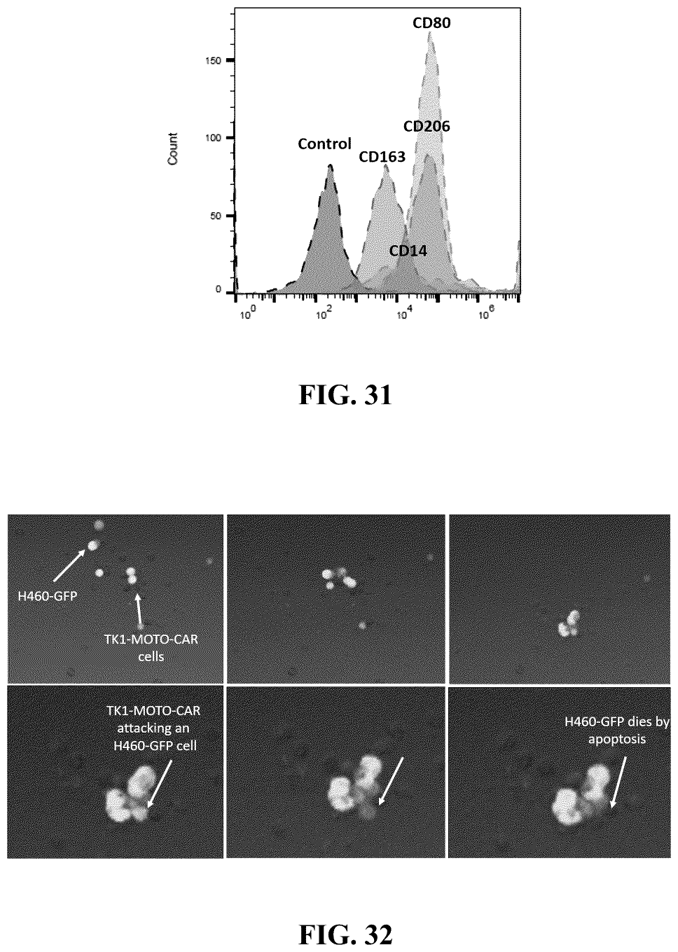

After day 10 of differentiation the transduction efficiency was assessed and macrophages expressing the chimeric receptor were cell sorted. Lentiviral transduction is challenging in macrophages. However, using HIV-1 based systems with EF1-α promoters almost 30% macrophage transduction was achieved. Transductions of the cells at early stages of macrophage differentiation displayed different transduction efficiencies. Monocytes or macrophages in earlier stages of differentiation are easier to transduce. Adenoviral transduction with the chimeric adenovirus AD5/F35 has emerged as another alternative for macrophage transduction. FIG. 29 shows the results of macrophages that were transduced being cell sorted using a FACSAria system. Around 30% of macrophage transduction was achieved using the lentiviral approach. The left most plot shows a control wherein only 0.58% of cells show fluorescence which would indicate expression of dTomato. The right two plots show a transduction efficiency of 27.1 percent after transduction.

### Example 6: Immunophenotyping of Transduced Macrophages

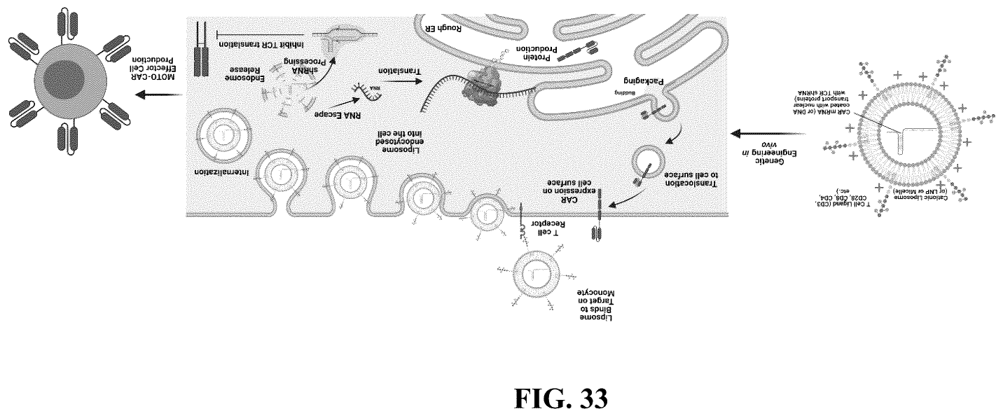

Immunophenotyping of macrophages transduced with vectors for the expression of a chimeric receptor was performed to identify the activation state of the transduced cells. It has been reported that modifications of the extracellular domain of TLR-4 may induce constant activation of its signaling domain (Gay et al, 2014). Constant activation of the TLR-4 signaling could lead to macrophage activation or M1 phenotype. It is not known if the construct which was used, which is based on TLR-4. is able to trigger a constant activation of the signaling through the TIR domain taken from TLR-4. However, after the transduction process, a change in the phenotype was observed and a change in the expression of cell surface markers in the macrophages. This is likely due to a combination of the lentiviral transduction and the expression of the chimeric receptor protein. Expression of CD14, CD80, D206 and low expression of CD163 were indicators of macrophage polarization towards the M1 phenotype. The expression of these cell surface markers in was observed in the transduced cells. FIG. 30 presents six scatter plots of fluorescence activated cell sorting demonstrating the retention of dye (Alexa 647), and the expression of CD80, CD163, CD206, and CD14 in macrophages transduced with a chimeric receptor.

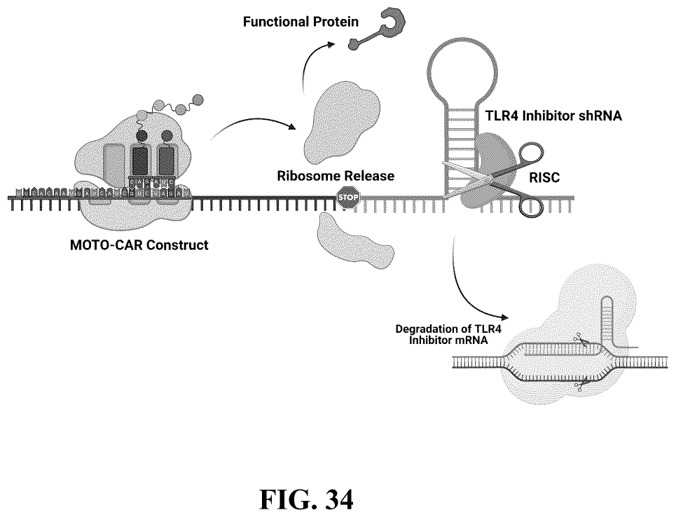

FIG. 31. Presents a histogram of the relative expression levels of M1 cells surface markers in macrophages transduced with a vector to express a chimeric receptor.

### Example 7: In-Vitro Toxicity of TK1 Targeting Chimeric Receptor Transduced Macrophages Against NCI-H460 Cells

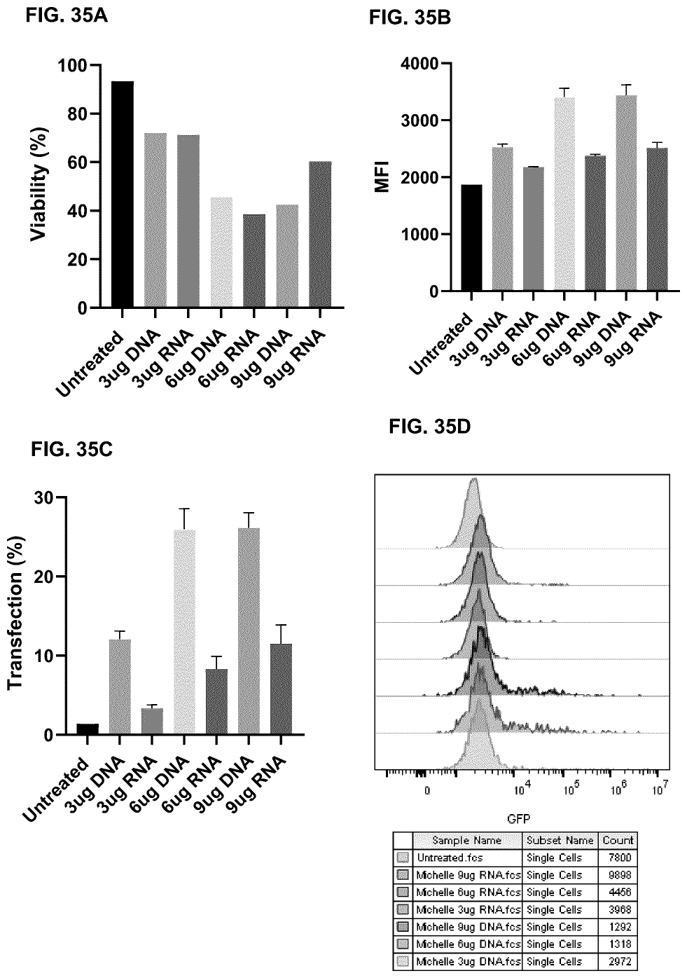

The tumoricidal activity of TK1 targeting chimeric receptor transduced macrophages was tested against NCI-H460-GFP cells. The E: T ratio used was 1:10. The analysis was performed with confocal microscopy. Detection of fluorescence was performed every 5 minutes during a 12 hour period. It was observed during time lapse that TK1 targeting chimeric receptor transduced macrophages migrate toward H460-GFP cells and attack them. After the synapsis, specific cell death is induced in the target cell. As demonstrated by the images in FIG. 32. TK1 targeting chimeric receptor transduced macrophages can interact with lung cancer cell lines expressing TK1. NCI-H460 cells were modified to express GFP. The activity of TK1 targeting chimeric receptor transduced macrophages was detected with confocal microscopy as a loss of fluorescence in the target cell.

### Example 8: Delivery of Nucleic Acid to a Target Cell

The delivery system of the present disclosure, will include, as a non-limiting example, a delivery vector, such as a cationic liposome (or LNP or Michelle), which will be carrying nucleic acid. In some embodiments the nucleic acid will be a chimeric antigen receptor mRNA, or a DNA with a nuclear transport protein as depicted in FIG. 33. In some embodiments the nucleic acid will also include a nucleic acid inhibitor that decreases the expression of proteins that form a part of the pathway that degrades the CAR in a mammalian cell, such as the TCR shRNA depicted in FIG. 33. The delivery vector will be targeted for specific cells, such as leukocytes, or particularly macrophages, or dendritic cells with a targeting agent on the surface of the delivery vector. In some embodiments this targeting agent will be a ligand for receptors on a macrophage or a dendritic cell, and in some embodiments may be a T cell ligand as depicted in FIG. 33. In some embodiments the targeting agent, such as a ligand, will bind to a receptor on the target cell, such as a receptor on a macrophage which will trigger endocytosis of the delivery vector into the cell. In some embodiments the vacuole formed by the endocytosis of the delivery vector will be processed releasing the nucleic acid the delivery vector was carrying. In some embodiments the nucleic acid and the nucleic acid inhibitor as depicted in FIG. 34 will be released into the cell. As a non-limiting example, the nucleic acid is RNA which will then be translated in the cell to produce a protein, which will be a chimeric antigen receptor (CAR). The CAR will then be processed in the cell and eventually expressed on the cell surface as depicted in FIG. 33. In some embodiments the nucleic acid inhibitor as depicted in FIG. 34 will inhibit, for example, TCR translation. In some embodiments, the CAR will also function as a receptor for a ligand on the delivery vector. In some embodiments when the ligand on the delivery vector binds to the CAR it will activate the cell. In some embodiments the cell activation will be to polarize a macrophage or dendritic cell into either an M1 or and M2 macrophage.

### Example 9: In Vitro Transfection with Mannose Coated Nanoparticles

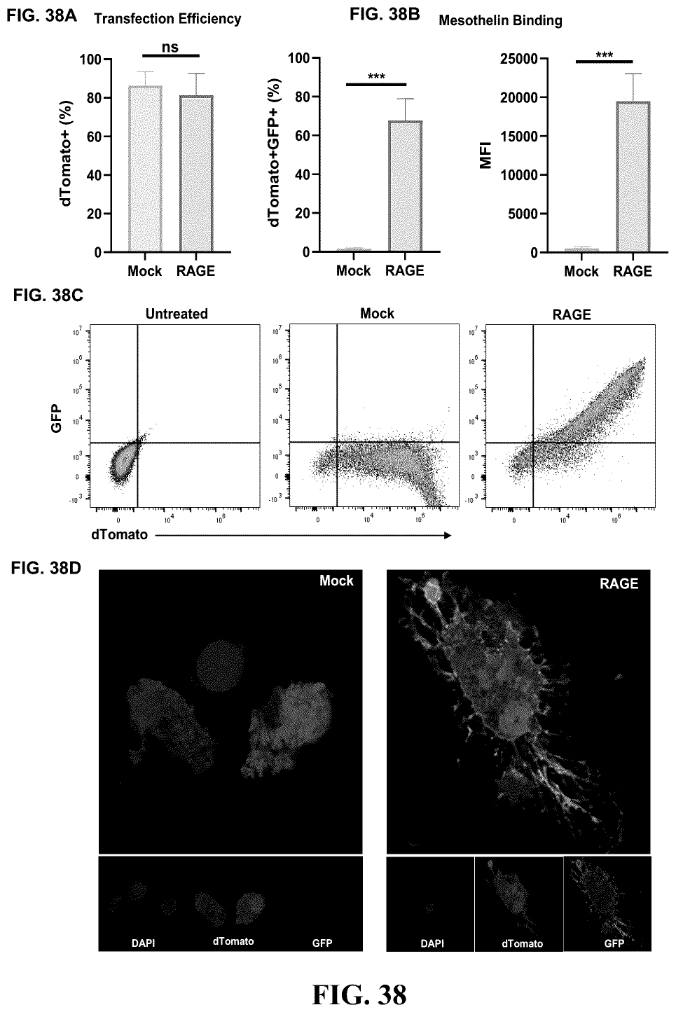

The delivery system of the present disclosure was used with targeted nanoparticle coated in mannose which were directed to macrophages. The nanoparticles contained various amounts of nucleic acid encoding GFP. In vitro, using various transfection parameters, the viability decreases upon nanoparticle transfection. FIG. 35A shows the viability percent (%) of cells in vitro for the different transfection parameters including the use of total nucleic acid applied to the cells of 3 μg DNA, 3 μg RNA, 6 μg RNA, 6 μg DNA, 9 μg DNA and 9 μg RNA all compared to an untreated sample. There is a viability above 60% for both the 3 μg DNA and 3 μg RNA; a near 60% viability for the 9 μg RNA; and an approximately 40% viability for 6 μg RNA, 6 μg DNA, and 9 μg DNA. The delivery system is shown to deliver various amounts of nucleic acid to cells and a significant portion of the cells so treated remained viable.

Once the cells were shown to be viable, the next step was to show that the transfection with the nanoparticles led to an incorporation of the nucleic acid into the cell and expression of the genes encoded therein by the cells. To that end, the different transfection parameters were examined for resulting mean fluorescence intensity (“MFI”) of the GFP to indicate the resulting expression for each of the parameters. FIG. 35B depicts MFI showing the relative intensity between the different treatments. For each of the different parameters there was a higher MFI than the untreated cells and for 6 μg DNA and 9 μg DNA there was a greater than 50% increase in MFI. We note that all samples display a normal “background” level of fluorescence depending on the cells used and instrument settings. The raw transfection efficiency is depicted in FIG. 35C shows the total transfection efficiency rate (%) in each condition of treatment. All conditions showed an increase in transfection efficiency over the untreated control with both 6 μg DNA and 9 μg DNA displaying the highest transfection percentage of well over 20%. FIG. 35D depicts a histogram overlay of GFP intensity of the various transfections when compared to untreated control.

Based on the results obtained in vitro through several runs, we have seen a high level of donor-to-donor variability that has an impact on the overall transfection and viability. Overall, we see the best results utilizing RNA as the vehicle for genetic delivery, but also see a relatively high success rate using 6 μg of DNA. Moving forward the standard for testing will be to utilize the 6 μg of DNA in vitro nanoparticle delivery.

### Example 10: In Vivo Transfection with Mannose Coated Nanoparticles

Following confirmation that the nanoparticles have the ability to transfect in vitro as seen in Example 9, evaluation of the ability of the nanoparticles to transfect in vivo was performed. This was done by dosing mice with nanoparticles for the delivery of a luciferase expression vector and evaluating their trafficking and expression in the immune competent BALB/c mouse model, depicted in FIG. 36A-B. Finding that the delivery system is capable of transfecting macrophages in mice with competent immune systems is an important step forward, and was unexpected. This shows that the delivery system can deliver a construct macrophages in a mouse that are localized to tumors in a mouse with a competent immune system. In the case of transfecting macrophages means that the transfected macrophage will either have been directed to and target the tumor cells or that the delivery system delivers the construct to macrophages already at the site of the tumor, such as TAMs. Such transfected macrophages can also utilize the entire intact immune system to target and attack tumor cells. In showing this transfection in vivo, the mice were either given a control, were given intraperitoneal (“IP”) injection with the delivery device, or were given an intravenous (“IV”) tail vein injection.

As is depicted in FIG. 36, in the 24 hrs of following injection, the mice were expressing a high level of luciferase indicating they had been successfully genetically engineered in vivo. The more exciting, and unexpected, component of this experiment can be visualized in the front views of the mice. In the IV injected mice was a significant level of luciferase signal from 3 areas of the mouse: tail. lungs. and tumor. It is extremely common for the nanoparticles to congregate in the lungs and also at the injection site, but the fascinating fact is the only other portion of the mouse that had a specific congregation of luciferase was in the tumor itself. This shows that tumors will draw in more nanoparticles due to the increase in overall angiogenesis and circulation that is required for sufficient tumor growth. This same observed localization to the tumor was also observed in the IP injections as well.

Again depicted in FIG. 36, following 48 hrs of incubation, the nanoparticles were not as bright and had lost a high amount of expression, but this may be inconsequential in our therapy as the effects of the engineering would have already been initiated.

This data shows that these directed nanoparticles do in vivo engineering and, more importantly, can engineer cells within the tumor itself in immune competent mice.

### Example 11: RAGE MOTO-CAR Evaluation in Human Monocytes

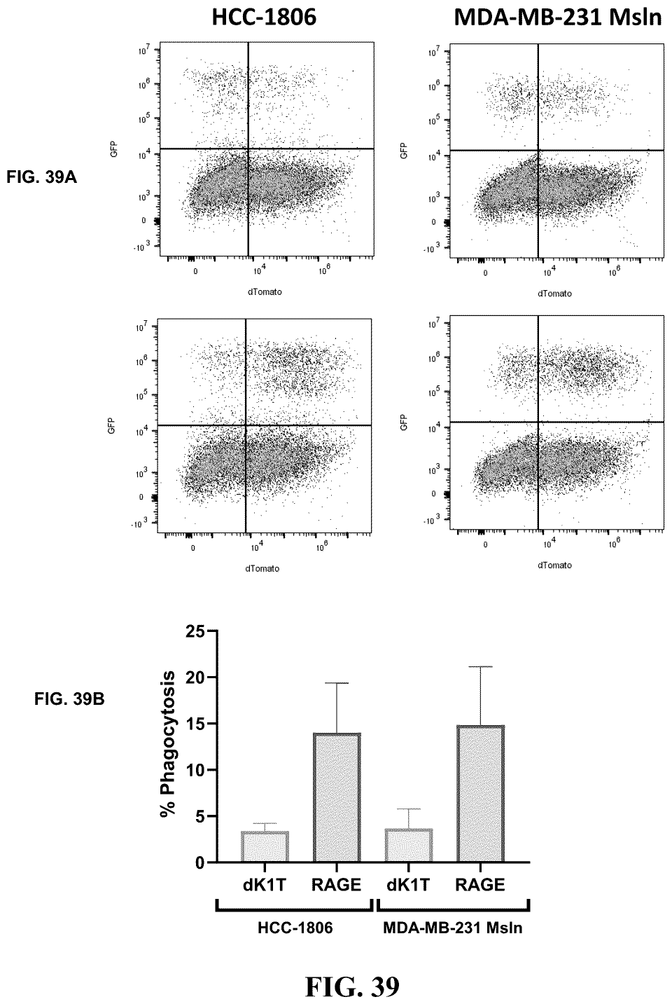

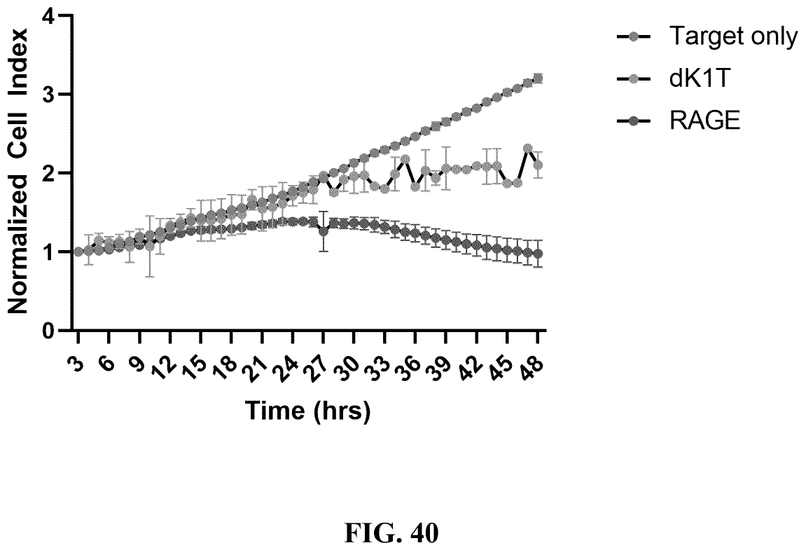

This disclosure will show both that the delivery system will transfect human monocytes, but also that the transfected monocyte will express a functional CAR. First RAGE MOTO-CARs were evaluated in primary human monocytes for both surface expression and target mesothelin protein binding. RAGE MOTO-CARs used herein contain an SS ScFv extracellular domain specific for mesothelin, as well as the intracellular domain of RAGE. The specific coding sequence used herein is presented in FIG. 37 which was linked via a IRES to a dTomato reporter. As depicted in FIG. 38A, the efficiency of the nanoparticle transfection was determined using dTomato+ reporter gene expression. RAGE MOTO-CAR and Mock transfected cells displayed a similar, insignificant difference in overall transfection efficiency. Next mesothelin binding was evaluated, as depicted in FIG. 38B, demonstrating surface translocation of the expressed CAR as evaluated through target protein binding. Mesothelin binding assayed by incubating the cells with GFP labelled mesothelin and binding was quantified using the dTomato+ subset of transfected cells to eliminate un-transfected contaminants. Mean fluorescent intensity (“MFI”) along with overall percentages were used for quantification. Overall, there was a significant elevation in mesothelin binding in cells nanoparticle transfected with the RAGE MOTO-CAR in comparison to the Mock. Qualitative visualization of fluorescent biding within each evaluated construct compared to untransfected controls is depicted in FIG. 38C. The untreated and mock trials show no correlation between the amount of dTomato and GFP while the RAGE MOTO-CAR shows a dose dependent relationship. The mesothelin binding is further confirmed through the qualitative observation of fluorescence through confocal microscopy. FIG. 38D depicts cells labeled with nuclear stain (DAPI), dTomato (transfection), and GFP (mesothelin). Mock cells showed no surface binding indicated by the lack of any GFP signal within the sample. In contrast, RAGE MOTO-CARs showed ubiquitous binding of the GFP/mesothelin conjugate along the perimeter of the cells demonstrating both surface localization of the expressed CAR as well as protein binding.

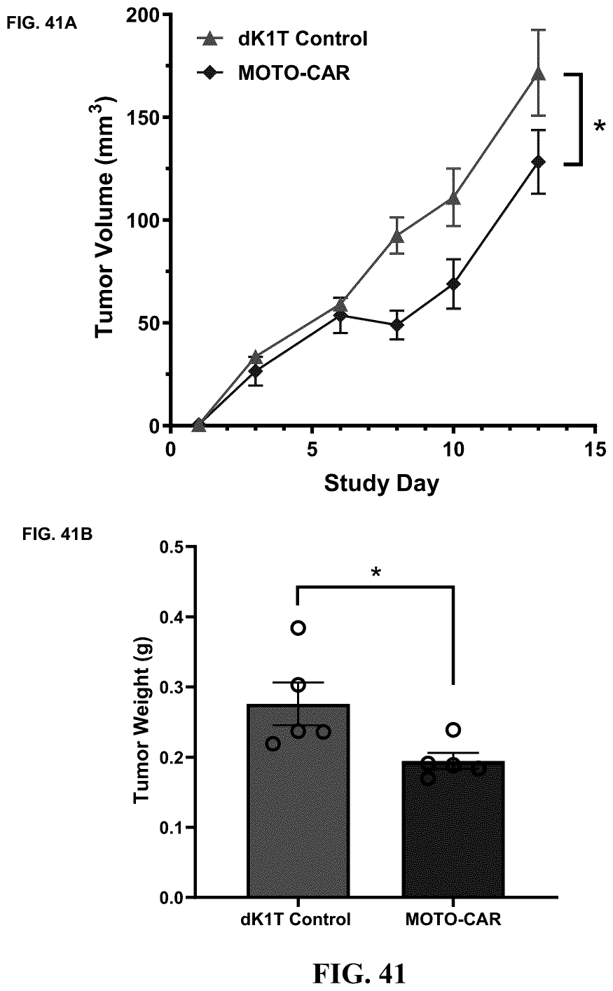

The ability of the nanoparticle transfected cells to phagocytose mesothelin positive and negative cells was investigated for primary RAGE MOTO-CARs. As is depicted in FIG. 39A, RAGE MOTO-CARs were exposed to a GFP expressing cell line that either lacked mesothelin expression (Msln−MDA-MB-231) or an version of the same cell line engineered to express human mesothelin (Msln+MDA-MB-231). To determine overall phagocytosis the cells were gated on live cells, followed by CD45+ cells and then were evaluated for transfection efficiency (dTomato). Phagocytosis would result in transfected cells (dTomato+) that are also GFP positive (having internalized a cell expressing GFP). Events were calculated by quantifying GFP+ events (target cells) in the dTomato+ (MOTO-CAR) population. Following co-incubation for 4 hours, there was a significant difference in overall phagocytosis between the cell lines. While co-incubation of transfected cells with mesothelin negative cells showed insignificant binding similar to mock controls, co-incubation of transfected cells with mesothelin positive cells resulted in a significant elevation of phagocytosis, as shown in the graph in FIG. 39B.

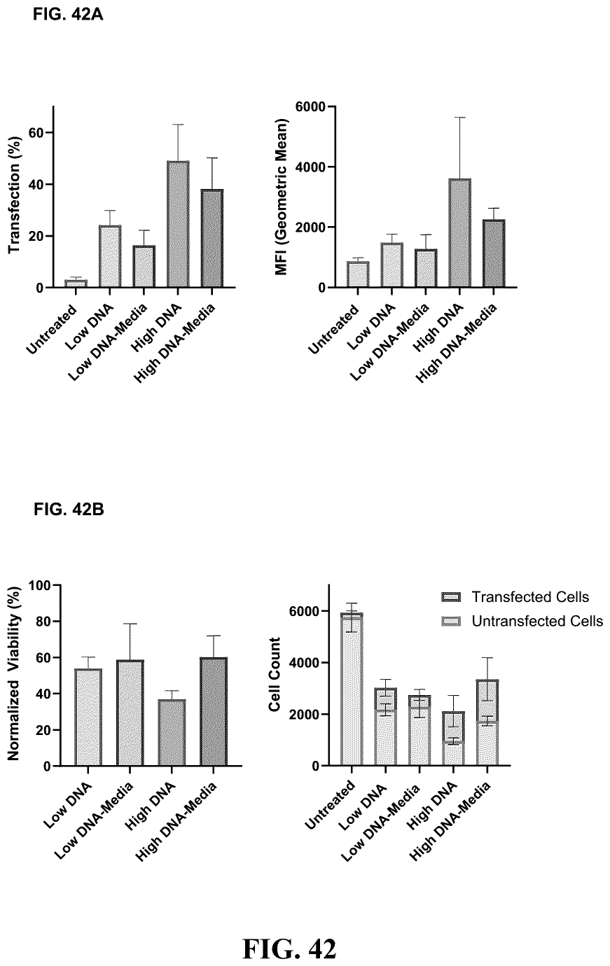

Once the successful transfection of the cells and functional phagocytosis was established, the targeted destruction of mesothelin expressing cells with CAR-T cells was investigated. As shown in FIGS. 38E and 38F, RAGE CAR-T cells preferentially killed target mesothelin positive HCC-1806 cells. T-cells were transduced with RAGE CAR and then further co-incubated with mesothelin expressing target cells. The overall target cell growth inhibition and relative cytolysis was evaluated via counting of remaining target cells over time. As depicted in FIG. 40, there was a significant reduction in the overall target cell population as measured by normalized cell index when co-incubated with RAGE CAR-T cells. This demonstrated that the RAGE CAR not only signals effectively in T cells, but also is able to elicit significant target cell killing independent of TCR signaling.

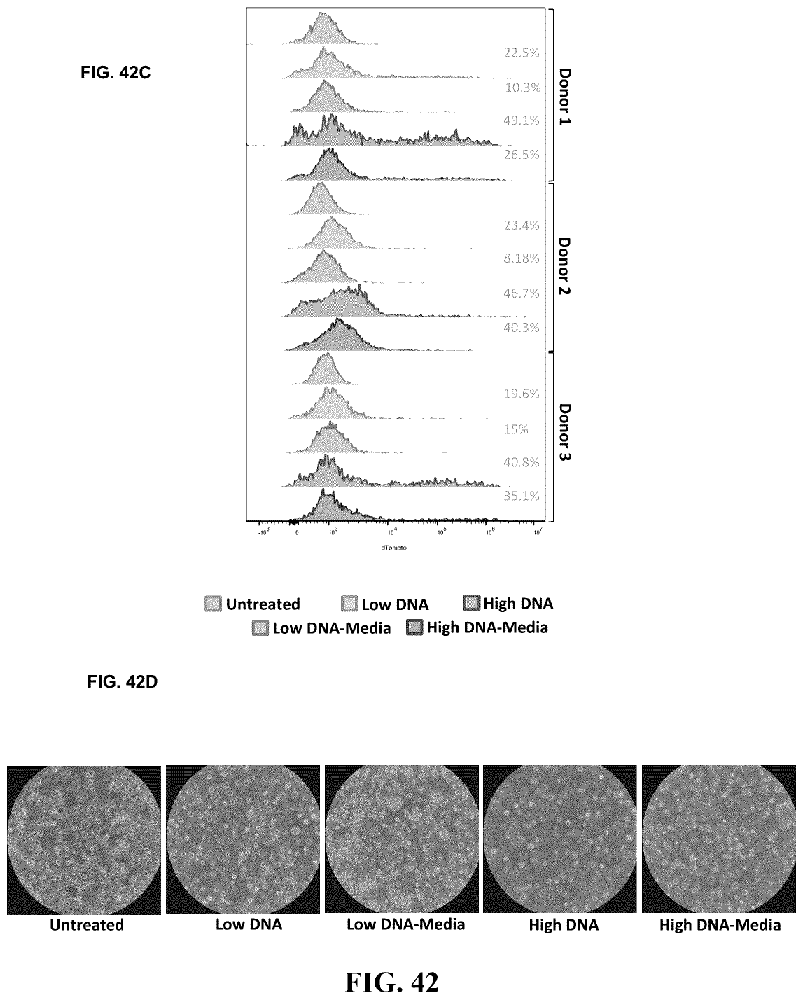

Finally, the RAGE MOTO-CARs were shown to reduce tumor burden in vivo. Following a co-injection with mesothelin positive HCC-1806 cells, the tumor burden in mice was measured over the time series depicted in FIG. 41A. Mice treated with the RAGE MOTO-CARs showed a significant reduction in both overall tumor weight, depicted in FIG. 41B, upon sacrifice as well as small volumetric measurements over the weeks of tumor growth. This was all accomplished with a single injection of the MOTO-CARS.

### Example 12: Transfection Efficiency

The nanoparticle transfection efficiency was examined using four different parameters: including Low DNA, Low DNA-Media, High DNA, and High DNA-Media in comparison to untreated cells. Low DNA=1 ug DNA/million cells. Low DNA-Media=1 ug DNA/million cells including an exchange to fresh media following 18 hrs of nanoparticle incubation. High DNA=2 ug DNA/million cells. High DNA-Media=2 ug DNA/million cells including an exchange to fresh media following 4 hrs of nanoparticle incubation.

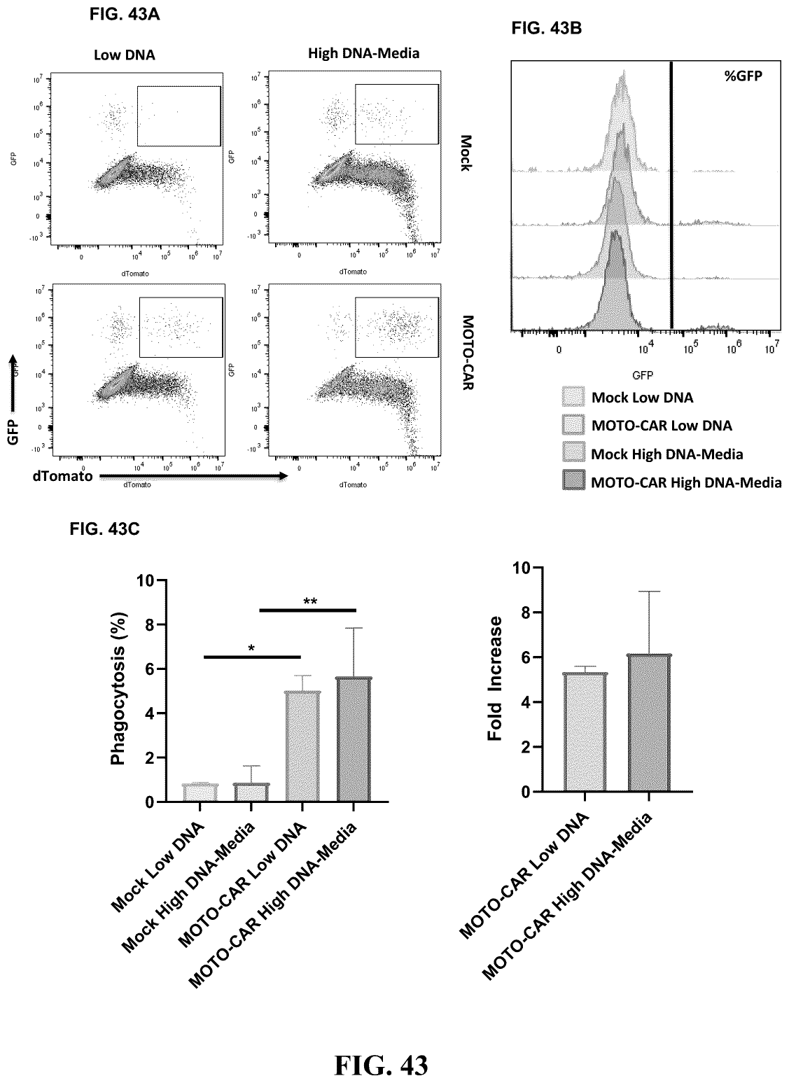

Cells were plated for 6 days in M-CSF to promote a high CD206 expression. Following growth, the cells were then washed with fresh media and the nanoparticles were added dropwise to the cells. Following nanoparticle addition, the cells were allowed to take up the nanoparticles for 4 hours in the case of the high DNA and 18 hours in the case of the low DNA. Then, media was removed and the cells were either grown further or evaluated for expression. The transfection percentage was highest in the High DNA and High DNA-media parameters, as depicted in FIG. 42A. In addition, the MFI (geometric mean) for the four parameters was compared to untreated cells. Again, the highest MFI was in the High DNA and the High DNA-Media assays.

Cell viability was examined after nanoparticle transfection as a function of normalized viability percentage (%), as shown in FIG. 42B. Each of the parameters, including Low DNA, Low DNA-Media, High DNA, and High DNA-Media, had normalized viability of close to 40% in the High DNA condition or above 50% in the other three conditions. The cell count for both transfected and untransfected cells is shown in FIG. 42B for each of the parameters and untreated cells. As expected, the untreated cells have no transfected cells. The number of transfected cells varies in the other four assays with the highest percent of transfected cells found in the High DNA and High DNA-Media assays. The percent of transfected cells was examined for three donors comparing untreated cells (control) to the four parameters, including Low DNA, Low DNA-Media, High DNA, and High DNA-Media. The highest percent of transfected was found in the High DNA assay in all three donors as depicted in FIG. 42 C. FIG. 42D displays micrographs of transfected cells of the four transfection parameters and untreated cells.

This data indicates that the best conditions to introduce DNA to cells among those tested were both the High DNA and High DNA-Media assays.

### Example 13: Target Cell Binding and Trogocytosis of Nanoparticle Transfected Macrophages

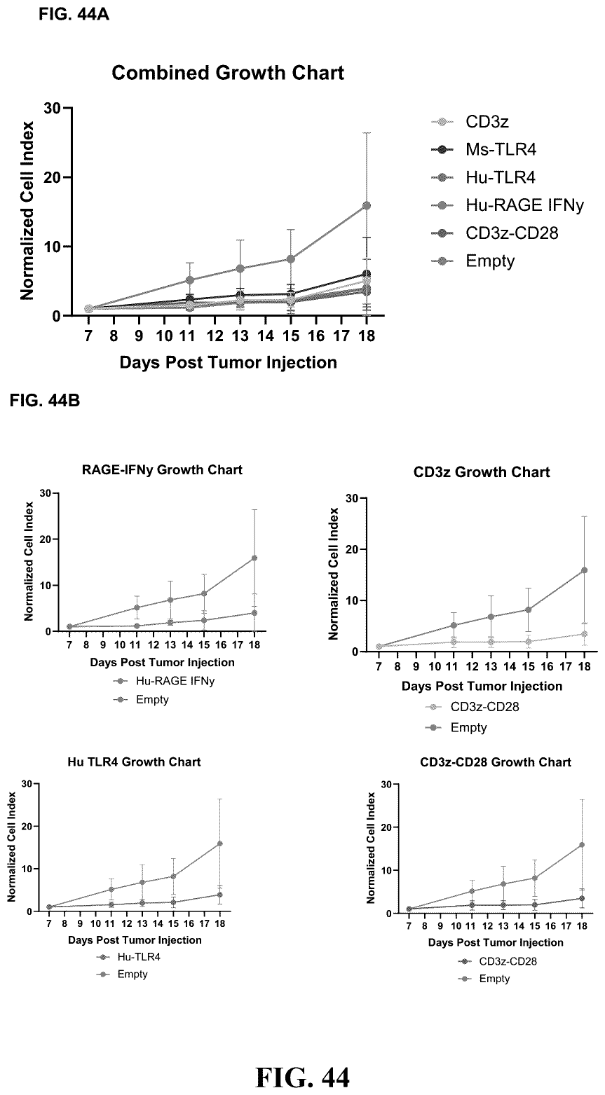

MOTO-CARs were expressed in primary macrophages via a nanoparticle transfection protocol and further co-cultured with GFP labeled target cancer cells for 4 hours. Following co-incubation, the cells were evaluated for double positive populations that indicate target cell binding and trogocytosis. Plots of the CD45+ population of effector cells and their relative transfection efficiency along with target cell binding are depicted in FIG. 43A for both Low DNA and High DNA-Media transfection protocols as described in Example 12. The MOTO-CAR transfected cells showed a significantly higher double positive population when compared to the mock control. The histogram depicted in FIG. 43B shows the relative double positivity between the controls and the MOTO-CAR assays. The quantification of the transfected cells that contain target cell GFP is depicted in FIG. 43C. Both the MOTO-CAR transfected cells have a significantly higher proportion of double positive populations than the mock controls. Both the MOTO-CAR transfected cells have an average six (6) fold increase in comparison to control also depicted in FIG. 43C.

### Example 14: Tumor Reduction Using Five Different CAR Constructs

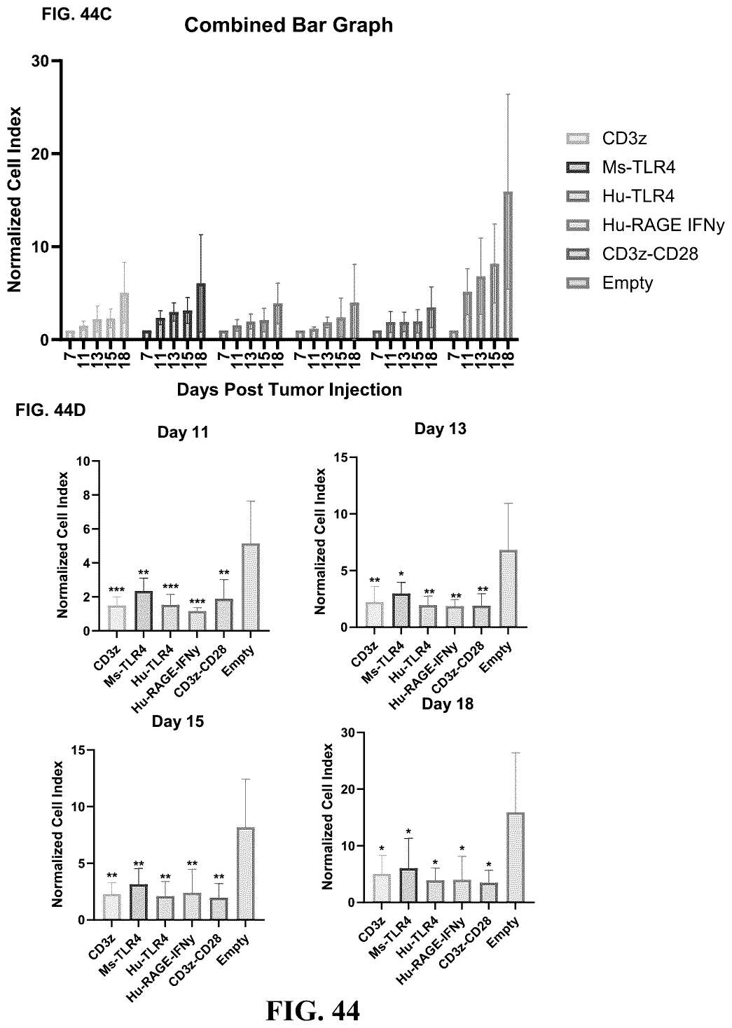

The transfection of macrophages was performed in BALB/c immune competent mice using an embodiment of nanoparticle delivery. The BALB/c mice were injected with human mesothelin expressing engineered 4T1 tumor cells and allowed to establish for 8 days. Following the 8 days of tumor growth, nanoparticles containing the CAR constructs were administered via an intravenous injection to the mice. The five CAR constructs used all had the same extracellular ScFv targeting domain, which was fused to a variety of cytoplasmic effect of domains. The cytoplasmic effector domains were CD35, MS-TLR4, Hu-TLR4, Hu-RAGE IFNγ, and CD3ζ-CD28. Empty nanoparticle lacking nucleic acids were used as a control. As shown in FIG. 44A, for each of the five constructs there was a reduction in the normalized cell index in comparison to control for the time series up to 18 days post tumor injection. FIG. 44B breaks out four of the constructs in comparison to control: Hu-RAGE IFNγ, CD3ζ, Hu-TLR4, CD3ζ-CD28. For each of these graphs there is a drastic decrease in tumor cells compared to control. FIG. 44C presents this data in a combined bar graph of normalized cell index at time points 7, 11, 13, 15, and 18 days post tumor injection for each of the CAR constructs: CD35, MS-TLR4, Hu-TLR4, Hu-RAGE IFNγ, CD32-CD28, and a control. FIG. 44D shows the individual bar graphs for day 11, 13, 15, and 18 for each of the CAR constructs and the control. The statistical significance was evaluated through a one-way ANOVA with the empty group (control) as the dedicated reference. Significance was designated by the following representation of p-values: *<0.05; **<0.01; ***<0.001; <0.0001.

This shows the unexpected result that each of the CAR constructs described in this disclosure was, via nanoparticle injection, ableto reduce tumor volume compared to control in an immune competent mammal. This has never been achieved before now.

The disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims and their legal equivalents.

TABLE OF REFERENCES

| | |
|---|---|
| 1. | Hanahan, D., & Weinberg, R. a. (2011). Hallmarks of cancer: the next generation. Cell, 144(5), |
| | 646-74. http://doi.org/10.1016/j.cell.2011.02.013 |
| 2. | American Cancer Society. (2015). Cancer Facts & Figures 2015. |
| 3. | Hoyert, D. L., & Xu, J. (2012). National Vital Statistics Reports Deaths: Preliminary Data |
| | for 2011 (Vol. 61). |
| 4. | Kurahara, H., Shinchi, H., Mataki, Y., Maemura, K., Noma, H., Kubo, F., . . . Takao, S. (2011). |
| | Significance of M2-polarized tumor-associated macrophage in pancreatic cancer. The Journal of |
| | Surgical Research, 167(2), e211-9. http://doi.org/10.1016/j.jss.2009.05.026 |
| 5. | Steidl, C., Lee, T., & Shah, S. (2010a). Tumor-associated macrophages and survival in classic |
| | Hodgkin's lymphoma. The New England Journal of Medicine, 875-885. Retrieved from |
| | http://www.nejm.org/doi/full/10.1056/NEJMoa0905680 |
| 6. | Eiró, N., & Vizoso, F. J. (2012). Inflammation and cancer. World Journal of Gastrointestinal |
| | Surgery, 4(3), 62-72. http://doi.org/10.4240/wjgs.v4.i3.62 |
| 7. | Kelly, P. M., Davison, R. S., Bliss, E., & McGee, J. O. (1988). Macrophages in human breast |
| | disease: a quantitative immunohistochemical study. British Journal of Cancer, 57(2), 174-7. |
| | Retrieved from |
| | http://www.pubmedcentral.nih.gov/articlerender.fcgi?artid=2246436&tool=pmcentrez&rendertype=abstract |
| 8. | Lewis, C., & Leek, R. (1995). Cytokine regulation of angiogenesis in breast cancer: the role of |
| | tumor-associated macrophages. Journal of Leukocyte . . . , 57(May), 747-751. Retrieved from |
| | http://www.jleukbio.org/content/57/5/747.short |
| 9. | Mantovani, A., Biswas, S. K., Galdiero, M. R., Sica, A., & Locati, M. (2013). Macrophage |
| | plasticity and polarization in tissue repair and remodelling. The Journal of Pathology, 229(2), |
| | 176-85. http://doi.org/10.1002/path.4133 |
| 10. | Porta, C., Rimoldi, M., Raes, G., Brys, L., Ghezzi, P., Di Liberto, D., . . . Sica, A. (2009). |
| | Tolerance and M2 (alternative) macrophage polarization are related processes orchestrated by |
| | p50 nuclear factor kappaB. Proceedings of the National Academy of Sciences of the United States |
| | of America, 106(35), 14978-83. http://doi.org/10.1073/pnas.0809784106 |
| 11. | Sica, A., & Mantovani, A. (2012). Macrophage plasticity and polarization: in vivo veritas. The |
| | Journal of Clinical Investigation, 122(3), 787-796. http://doi.org/10.1172/JCI59643DS1 |
| 12. | Anderson, C. F., & Mosser, D. M. (2002). A novel phenotype for an activated macrophage: |
| | the type 2 activated macrophage. Journal of Leukocyte Biology, 72(1), 101-6. Retrieved from |
| | http://www.ncbi.nlm.nih.gov/pubmed/12101268 |
| 13. | Ghassabeh, G. H., De Baetselier, P., Brys, L., Noël, W., Van Ginderachter, J. a, Meerschaut, |
| | S., . . . Raes, G. (2006). Identification of a common gene signature for type II cytokine- |
| | associated myeloid cells elicited in vivo in different pathologic conditions. Blood, 108(2), |
| | 575-83. http://doi.org/10.1182/blood-2005-04-1485 |
| 14. | Liao, X., Sharma, N., & Kapadia, F. (2011). Krüppel-like factor 4 regulates macrophage |
| | polarization. The Journal of Clinical Investigation, 121(7). http://doi.org/10.1172/JCI45444DS1 |
| 15. | Davis, M. J., Tsang, T. M., Qiu, Y., Dayrit, J. K., Freij, J. B., Huffnagle, G. B., & Olszewski, |
| | M. A. (2013). Macrophage M1/M2 polarization dynamically adapts to changes in cytokine |
| | microenvironments in Cryptococcus neoformans infection. mBio, 4(3), e00264-13. |
| | http://doi.org/10.1128/mBio.00264-13 |
| 16. | Mantovani, A., Sozzani, S., Locati, M., Allavena, P., & Sica, A. (2002). Macrophage |
| | polarization: tumor-associated macrophages as a paradigm for polarized M2 mononuclear |
| | phagocytes. Trends in Immunology, 23(11), 549-55. Retrieved from |
| | http://www.ncbi.nlm.nih.gov/pubmed/12401408 |
| 17. | Edin, S., Wikberg, M. L., Dahlin, A. M., Rutegård, J., Oberg, A., Oldenborg, P.-A., & Palmqvist, |
| | R. (2012). The distribution of macrophages with a m1 or m2 phenotype in relation to prognosis |
| | and the molecular characteristics of colorectal cancer. PloS One, 7(10), e47045. |
| | http://doi.org/10.1371/journal.pone.0047045 |
| 18. | Forssell, J., Oberg, A., Henriksson, M. L., Stenling, R., Jung, A., & Palmqvist, R. (2007). |
| | High macrophage infiltration along the tumor front correlates with improved survival in colon |
| | cancer. Clinical Cancer Research, 13(5), 1472-9. http://doi.org/10.1158/1078-0432.CCR-06- |
| | 2073 |
| 19. | Guiducci, C., Vicari, A. P., Sangaletti, S., Trinchieri, G., & Colombo, M. P. (2005). |
| | Redirecting in vivo elicited tumor infiltrating macrophages and dendritic cells towards tumor |
| | rejection. Cancer Research, 65(8), 3437-46. http://doi.org/10.1158/0008-5472.CAN-04- |
| | 4262 |
| 20. | Baccala, R., Hoebe, K., Kono, D. H., Beutler, B., & Theofilopoulos, A. N. (2007). TLR- |
| | dependent and TLR-independent pathways of type I interferon induction in systemic |
| | autoimmunity. Nature Medicine, 13(5), 543-51. http://doi.org/10.1038/nm1590 |
| 21. | Banerjee, S., Xie, N., Cui, H., Tan, Z., Yang, S., Icyuz, M., . . . Liu, G. (2013). MicroRNA let-7c |
| | regulates macrophage polarization. Journal of Immunology (Baltimore, Md.: 1950), 190(12), |
| | 6542-9. http://doi.org/10.4049/jimmunol.1202496 |
| 22. | Murray, P. J., Allen, J. E., Biswas, S. K., Fisher, E. A., Gilroy, D. W., Goerdt, S., . . . Wynn, T. |
| | A. (2014). Macrophage Activation and Polarization: Nomenclature and Experimental |
| | Guidelines. Immunity, 41(1), 14-20. http://doi.org/10.1016/j.immuni.2014.06.008 |
| 23. | Hao, N.-B., Lü, M.-H., Fan, Y.-H., Cao, Y.-L., Zhang, Z.-R., & Yang, S.-M. (2012). |
| | Macrophages in tumor microenvironments and the progression of tumors. Clinical & |
| | Developmental Immunology, 2012, 948098. http://doi.org/10.1155/2012/948098 |
| 24. | Sinha, P., Clements, V. K., & Ostrand-Rosenberg, S. (2005). Reduction of myeloid-derived |
| | suppressor cells and induction of M1 macrophages facilitate the rejection of established |
| | metastatic disease. Journal of Immunology, 174(2), 636-45. Retrieved from |
| | http://www.ncbi.nlm.nih.gov/pubmed/15634881 |
| 25. | Bingle, L., Brown, N. J., & Lewis, C. E. (2002). The role of tumour-associated macrophages in |
| | tumour progression: implications for new anticancer therapies. The Journal of Pathology, 196(3), |
| | 254-65. http://doi.org/10.1002/path.1027 |
| 26. | Herbeuval, J.-P., Lambert, C., Sabido, O., Cottier, M., Fournel, P., Dy, M., & Genin, C. |
| | (2003). Macrophages from cancer patients: analysis of TRAIL, TRAIL receptors, and colon |
| | tumor. Journal of the National Cancer Institute, 95(8), 611-21. Retrieved from |
| | http://www.ncbi.nlm.nih.gov/pubmed/12697854 |
| 27. | Ma, J., Liu, L., Che, G., Yu, N., Dai, F., & You, Z. (2010). The M1 form of tumor-associated |
| | macrophages in non-small cell lung cancer is positively associated with survival time. BMC |
| | Cancer, 10, 112. http://doi.org/10.1186/1471-2407-10-112 |
| 28. | Ohri, C. M., Shikotra, A., Green, R. H., Waller, D. a, & Bradding, P. (2009). Macrophages within |
| | NSCLC tumour islets are predominantly of a cytotoxic M1 phenotype associated with extended |
| | survival. The European Respiratory Journal, 33(1), 118-26. |
| | http://doi.org/10.1183/09031936.00065708 |
| 29. | Urban, J. L., Shepard, H. M., Rothstein, J. L., Sugarman, B. J., & Schreiber, H. (1986). Tumor |
| | necrosis factor: a potent effector molecule for tumor cell killing by activated macrophages. |
| | Proceedings of the National Academy of Sciences of the United States of America, 83(14), 5233- |
| | 7. Retrieved from |
| | http://www.pubmedcentral.nih.gov/articlerender.fcgi?artid=323925&tool-pmcentrez&rendertype=abstract |
| 30. | Wong, S.-C., Puaux, A.-L., Chittezhath, M., Shalova, I., Kajiji, T. S., Wang, X., . . . Biswas, S. |
| | K. (2010). Macrophage polarization to a unique phenotype driven by B cells. European Journal |
| | of Immunology, 40(8), 2296-307. http://doi.org/10.1002/eji.200940288 |
| 31. | Hardison, S. E., Herrera, G., Young, M. L., Hole, C. R., Wozniak, K. L., & Wormley, F. L. |
| | (2012). Protective immunity against pulmonary cryptococcosis is associated with STAT1- |
| | mediated classical macrophage activation. Journal of Immunology (Baltimore, Md.: 1950), |
| | 189(8), 4060-8. http://doi.org/10.4049/jimmunol.1103455 |
| 32. | Wang, Y.-C., He, F., Feng, F., Liu, X.-W., Dong, G.-Y., Qin, H.-Y., . . . Han, H. (2010). Notch |
| | signaling determines the M1 versus M2 polarization of macrophages in antitumor immune |
| | responses. Cancer Research, 70(12), 4840-9. http://doi.org/10.1158/0008-5472.CAN-10-0269 |
| 33. | Cai, X., Yin, Y., Li, N., Zhu, D., Zhang, J., Zhang, C.-Y., & Zen, K. (2012). Re-polarization of |
| | tumor-associated macrophages to pro-inflammatory M1 macrophages by microRNA-155. |
| | Journal of Molecular Cell Biology, 4(5), 341-3. http://doi.org/10.1093/jmcb/mjs044 |
| 34. | Wei, Y., Nazari-Jahantigh, M., Chan, L., Zhu, M., Heyll, K., Corbalán-Campos, J., . . . Schober, |
| | A. (2013). The microRNA-342-5p fosters inflammatory macrophage activation through an Akt1- |
| | and microRNA-155-dependent pathway during atherosclerosis. Circulation, 127(15), 1609-19. |
| | http://doi.org/10.1161/CIRCULATIONAHA.112.000736 |
| 35. | Squadrito, M. L., Etzrodt, M., De Palma, M., & Pittet, M. J. (2013). MicroRNA-mediated |
| | control of macrophages and its implications for cancer. Trends in Immunology, 34(7), 350-9. |
| | http://doi.org/10.1016/j.it.2013.02.003 |
| 36. | Biswas, S. K., Gangi, L., Paul, S., Schioppa, T., Saccani, A., Sironi, M., . . . Sica, A. (2006). A |
| | distinct and unique transcriptional program expressed by tumor-associated macrophages |
| | (defective NF-kappaB and enhanced IRF-3/STAT1 activation). Blood, 107(5), 2112-22. |
| | http://doi.org/10.1182/blood-2005-01-0428 |
| 37. | Steidl, C., Lee, T., & Shah, S. (2010b). Tumor-associated macrophages and survival in classic |
| | Hodgkin's lymphoma. The New England Journal of Medicine, 362(10), 875-885. Retrieved |
| | from http://www.nejm.org/doi/full/10.1056/NEJMoa0905680 |
| 38. | Lin, E. Y., Li, J.-F., Gnatovskiy, L., Deng, Y., Zhu, L., Grzesik, D. a, . . . Pollard, J. W. (2006). |
| | Macrophages regulate the angiogenic switch in a mouse model of breast cancer. Cancer |
| | Research, 66(23), 11238-46. http://doi.org/10.1158/0008-5472.CAN-06-1278 |
| 39. | Hagemann, T., Wilson, J., Burke, F., Kulbe, H., Li, N. F., Plüddemann, A., . . . Balkwill, F. R. |
| | (2006). Ovarian cancer cells polarize macrophages toward a tumor-associated phenotype. The |
| | Journal of Immunology, 176(8), 5023-32. Retrieved from |
| | http://www.ncbi.nlm.nih.gov/pubmed/16585599 |
| 40. | Hagemann, T., Lawrence, T., McNeish, I., Charles, K. a, Kulbe, H., Thompson, R. G., . . . |
| | Balkwill, F. R. (2008). “Re-educating” tumor-associated macrophages by targeting NF- |
| | kappaB. The Journal of Experimental Medicine, 205(6), 1261-8. |
| | http://doi.org/10.1084/jem.20080108 |
| 41. | Mandal, P., Pratt, B. T., Barnes, M., McMullen, M. R., & Nagy, L. E. (2011). Molecular |
| | mechanism for adiponectin-dependent M2 macrophage polarization: link between the metabolic |
| | and innate immune activity of full-length adiponectin. The Journal of Biological Chemistry, |
| | 286(15), 13460-9. http://doi.org/10.1074/jbc.M110.204644 |
| 42. | Mantovani, A., Allavena, P., Sica, A., & Balkwill, F. (2008). Cancer-related inflammation. |
| | Nature, 454(7203), 436-44. http://doi.org/10.1038/nature07205 |
| 43. | Cortez-Retamozo, V., Etzrodt, M., Newton, A., Rauch, P. J., Chudnovskiy, A., Berger, C., . . . |
| | Pittet, M. J. (2012). Origins of tumor-associated macrophages and neutrophils. Proceedings of |
| | the National Academy of Sciences of the United States of America, 109(7), 2491-6. |
| | http://doi.org/10.1073/pnas.1113744109 |
| 44. | Hercus, T. R., Thomas, D., Guthridge, M. A., Ekert, P. G., King-Scott, J., Parker, M. W., & |
| | Lopez, A. F. (2009). The granulocyte-macrophage colony-stimulating factor receptor: linking |
| | its structure to cell signaling and its role in disease. Blood, 114(7), 1289-98. |
| | http://doi.org/10.1182/blood-2008-12-164004 |
| 45. | Smith, H. O., Stephens, N. D., Qualls, C. R., Fligelman, T., Wang, T., Lin, C.-Y., . . . Pollard, J. |
| | W. (2013). The clinical significance of inflammatory cytokines in primary cell culture in |
| | endometrial carcinoma. Molecular Oncology, 7(1), 41-54. |
| | http://doi.org/10.1016/j.molonc.2012.07.002 |
| 46. | West, R. B., Rubin, B. P., Miller, M. A., Subramanian, S., Kaygusuz, G., Montgomery, K., . . . |
| | van de Rijn, M. (2006). A landscape effect in tenosynovial giant-cell tumor from activation |
| | of CSF1 expression by a translocation in a minority of tumor cells. Proceedings of the |
| | National Academy of Sciences of the United States of America, 103(3), 690-5. |
| | http://doi.org/10.1073/pnas.0507321103 |
| 47. | Lin, E. Y., & Pollard, J. W. (2007). Tumor-associated macrophages press the angiogenic |
| | switch in breast cancer. Cancer Research, 67(11), 5064-6. http://doi.org/10.1158/0008- |
| | 5472.CAN-07-0912 |
| 48. | Dalton, H. J., Armaiz-Pena, G. N., Gonzalez-Villasana, V., Lopez-Berestein, G., Bar-Eli, M., & |
| | Sood, A. K. (2014). Monocyte subpopulations in angiogenesis. Cancer Research, 74(5), 1287- |
| | 93. http://doi.org/10.1158/0008-5472.CAN-13-2825 |
| 49. | Saccani, A., Schioppa, T., Porta, C., Biswas, S. K., Nebuloni, M., Vago, L., . . . Sica, A. (2006). |
| | p50 nuclear factor-kappaB overexpression in tumor-associated macrophages inhibits M1 |
| | inflammatory responses and antitumor resistance. Cancer Research, 66(23), 11432-40. |
| | http://doi.org/10.1158/0008-5472.CAN-06-1867 |
| 50. | Gazzaniga, S., Bravo, A. I., Guglielmotti, A., van Rooijen, N., Maschi, F., Vecchi, A., . . . |
| | Wainstok, R. (2007). Targeting tumor-associated macrophages and inhibition of MCP-1 reduce |
| | angiogenesis and tumor growth in a human melanoma xenograft. The Journal of Investigative |
| | Dermatology, 127(8), 2031-41. http://doi.org/10.1038/sj.jid.5700827 |
| 51. | Luo, Y., Zhou, H., & Krueger, J. (2006). Targeting tumor-associated macrophages as a novel |
| | strategy against breast cancer. Journal of Clinical Investigation, 116(8), 2132-2141. |
| | http://doi.org/10.1172/JCI27648.2132 |
| 52. | Zeisberger, S. M., Odermatt, B., Marty, C., Zehnder-Fjällman, a H. M., Ballmer-Hofer, K., & |
| | Schwendener, R. a. (2006). Clodronate-liposome-mediated depletion of tumour-associated |
| | macrophages: a new and highly effective antiangiogenic therapy approach. British Journal of |
| | Cancer, 95(3), 272-81. http://doi.org/10.1038/sj.bjc.6603240 |
| 53. | Bettencourt-Dias, M., Giet, R., Sinka, R., Mazumdar, a, Lock, W. G., Balloux, F., . . . Glover, D. |
| | M. (2004). Genome-wide survey of protein kinases required for cell cycle progression. Nature, |
| | 432(7020), 980-7. http://doi.org/10.1038/nature03160 |
| 54. | Geschwind, J. H., Vali, M., & Wahl, R. (2006). Effects of 3 bromopyruvate (hexokinase 2 |
| | inhibitor) on glucose uptake in lewis rats using 2-(F-18) fluoro-2-deoxy-d-glucose. In 2006 |
| | Gastrointestinal Cancers Symposium (pp. 12-14). |
| 55. | Wolf, A., Agnihotri, S., Micallef, J., Mukherjee, J., Sabha, N., Cairns, R., . . . Guha, A. (2011). |
| | Hexokinase 2 is a key mediator of aerobic glycolysis and promotes tumor growth in human |
| | glioblastoma multiforme. The Journal of Experimental Medicine, 208(2), 313-26. |
| | http://doi.org/10.1084/jem.20101470 |
| 56. | Blagih, J., & Jones, R. G. (2012). Polarizing macrophages through reprogramming of glucose |
| | metabolism. Cell Metabolism, 15(6), 793-5. http://doi.org/10.1016/j.cmet.2012.05.008 |
| 57. | Haschemi, A., Kosma, P., Gille, L., Evans, C. R., Burant, C. F., Starkl, P., . . . Wagner, O. |
| | (2012). The sedoheptulose kinase CARKL directs macrophage polarization through control |
| | of glucose metabolism. Cell Metabolism, 15(6), 813-26. |
| | http://doi.org/10.1016/j.cmet.2012.04.023 |
| 58. | Arranz, A., Doxaki, C., Vergadi, E., Martinez de la Torre, Y., Vaporidi, K., Lagoudaki, E. |
| | D., . . . Tsatsanis, C. (2012). Akt1 and Akt2 protein kinases differentially contribute to |
| | macrophage polarization. Proceedings of the National Academy of Sciences of the United |
| | States of America, 109(24), 9517-22. http://doi.org/10.1073/pnas.1119038109 |
| 59. | Jones, R. G., & Thompson, C. B. (2007). Revving the engine: signal transduction fuels T cell |
| | activation. Immunity, 27(2), 173-8. http://doi.org/10.1016/j.immuni.2007.07.008 |
| 60. | Shu, C. J., Guo, S., Kim, Y. J., Shelly, S. M., Nijagal, A., Ray, P., . . . Witte, O. N. (2005). |
| | Visualization of a primary anti-tumor immune response by positron emission tomography. |
| | Proceedings of the National Academy of Sciences of the United States of America, 102(48), |
| | 17412-7. http://doi.org/10.1073/pnas.0508698102 |
| 61. | Van Ginderachter, J. A., Movahedi, K., Hassanzadeh Ghassabeh, G., Meerschaut, S., Beschin, |
| | A., Raes, G., & De Baetselier, P. (2006). Classical and alternative activation of mononuclear |
| | phagocytes: Picking the best of both worlds for tumor promotion. Immunobiology, 211(6), 487- |
| | 501. Retrieved from http://www.sciencedirect.com/science/article/pii/S0171298506000829 |
| 62. | Mills, C. D., Shearer, J., Evans, R., & Caldwell, M. D. (1992). Macrophage arginine metabolism |
| | and the inhibition or stimulation of cancer. Journal of Immunology (Baltimore, Md.: 1950), |
| | 149(8), 2709-14. Retrieved from http://www.ncbi.nlm.nih.gov/pubmed/1401910 |
| 63. | Ji, Y., Sun, S., Xu, A., Bhargava, P., Yang, L., Lam, K. S. L., . . . Qi, L. (2012). Activation of |
| | natural killer T cells promotes M2 Macrophage polarization in adipose tissue and improves |
| | systemic glucose tolerance via interleukin-4 (IL-4)/STAT6 protein signaling axis in obesity. The |
| | Journal of Biological Chemistry, 287(17), 13561-71. http://doi.org/10.1074/jbc.M112.350066 |
| 64. | Andreesen, R., Scheibenbogen, C., & Brugger, W. (1990). Adoptive transfer of tumor cytotoxic |
| | macrophages generated in vitro from circulating blood monocytes: a new approach to cancer |
| | immunotherapy. Cancer Research, 7450-7456. Retrieved from |
| | http://cancerres.aacrjournals.org/content/50/23/7450.short |
| 65. | Korbelik, M., Naraparaju, V. R., & Yamamoto, N. (1997). Macrophage-directed |
| | immunotherapy as adjuvant to photodynamic therapy of cancer. British Journal of Cancer, |
| | 75(2), 202-7. Retrieved from |
| | http://www.pubmedcentral.nih.gov/articlerender.fcgi?artid=2063270&tool=pmcentrez&rendertype=abstract |
| 66. | Ellem, K. A. O., Rourke, M. G. E. O., Johnson, G. R., Parry, G., Misko, I. S., Schmidt, C. W., . . . |
| | Mulligan, R. C. (1997). A case report: immune responses and clinical course of the first human |
| | use of granulocyte/macrophage-colony-stimulating-factor-transduced autologous melanoma. |
| | Cancer Immunology, Immunotherapy, 10-20. Retrieved from |
| | http://www.springerlink.com/index/JQ4EB21E4C7ADMT7.pdf |
| 67. | Gast, G. de, & Klümpen, H. (2000). immunotherapy with subcutaneous granulocyte macrophage |
| | colony-stimulating factor, low-dose interleukin 2, and interferon Î± in progressive metastatic |
| | melanoma. Clinical Cancer Research. Retrieved from |
| | http://clincancerres.aacrjournals.org/content/6/4/1267.short |
| 68. | Hill, H., Jr, T. C., & Sabel, M. (2002). Immunotherapy with Interleukin 12 and Granulocyte- |
| | Macrophage Colony-stimulating Factor-encapsulated Microspheres Coinduction of Innate and |
| | Adaptive Antitumor. Cancer Research. Retrieved from |
| | http://cancerres.aacrjournals.org/content/62/24/7254.short |
| 69. | Lokshin, A., Mayotte, J., & Levitt, M. (1995). Mechanism of Interferon Beta-Induced Squamous |
| | Differentiation and Programmed Cell Death in Human Non-Small-Cell Lung Cancer Cell Lines. |
| | Journal of the National Cancer Institute, 87, 206-212. Retrieved from |
| | http://jnci.oxfordjournals.org/content/87/3/206.short |
| 70. | Johns, T., & Mackay, I. (1992). Antiproliferative potencies of interferons on melanoma cell |
| | lines and xenografts: higher efficacy of interferon Î². Journal of the National Cancer Institute, |
| | (type II), 1185-1190. Retrieved from http://jnci.oxfordjournals.org/content/84/15/1185 |
| 71. | Qin, X.-Q., Runkel, L., Deck, C., DeDios, C., & Barsoum, J. (1997). Interferon-beta induces S |
| | phase accumulation selectively in human transformed cells. Journal of Interferon & Cytokine |
| | Research, 17(6), 355-367. http://doi.org/10.1089/jir.1997.17.355 |
| 72. | Zhang, F., Lu, W., & Dong, Z. (2002). Tumor-infiltrating macrophages are involved in |
| | suppressing growth and metastasis of human prostate cancer cells by INF-β gene therapy in nude |
| | mice. Clinical Cancer Research, 2942-2951. Retrieved from |
| | http://clincancerres.aacrjournals.org/content/8/9/2942.short |
| 73. | Simpson, K. D., Templeton, D. J., & Cross, J. V. (2012). Macrophage Migration Inhibitory |
| | Factor Promotes Tumor Growth and Metastasis by Inducing Myeloid-Derived Suppressor |
| | Cells in the Tumor Microenvironment. The Journal of Immunology. |
| | http://doi.org/10.4049/jimmunol.1201161 |
| 74. | Sanford, D. E., Belt, B. A., Panni, R. Z., Mayer, A., Deshpande, A. D., Carpenter, D., . . . |
| | Linehan, D. C. (2013). Inflammatory monocyte mobilization decreases patient survival in |
| | pancreatic cancer: a role for targeting the CCL2/CCR2 axis. Clinical Cancer Research: An |
| | Official Journal of the American Association for Cancer Research, 19(13), 3404-15. |
| | http://doi.org/10.1158/1078-0432.CCR-13-0525 |
| 75. | Schmall, A., Al-Tamari, H. M., Herold, S., Kampschulte, M., Weigert, A., Wietelmann, A., . . . |
| | Savai, R. (2014). Macrophage and Cancer Cell Crosstalk via CCR2 and CX3CR1 is a |
| | Fundamental Mechanism Driving Lung Cancer. American Journal of Respiratory and |
| | Critical Care Medicine. http://doi.org/10.1164/rccm.201406-1137OC |
| 76. | Kimura, Y. N., Watari, K., Fotovati, A., Hosoi, F., Yasumoto, K., Izumi, H., . . . Ono, M. |
| | (2007). Inflammatory stimuli from macrophages and cancer cells synergistically promote |
| | tumor growth and angiogenesis. Cancer Science, 98(12), 2009-18. |
| | http://doi.org/10.1111/j.1349-7006.2007.00633.x |
| 77. | Chen, H., Li, P., Yin, Y., Cai, X., Huang, Z., Chen, J., . . . Zhang, J. (2010). The promotion of |
| | type 1 T helper cell responses to cationic polymers in vivo via toll-like receptor-4 mediated |
| | IL-12 secretion. Biomaterials, 31(32), 8172-80. |
| | http://doi.org/10.1016/j.biomaterials.2010.07.056 |
| 78. | Rogers, T. L., & Holen, I. (2011). Tumour macrophages as potential targets of |
| | bisphosphonates. Journal of Translational Medicine, 9(1), 177. http://doi.org/10.1186/1479- |
| | 5876-9-177 |
| 79. | Junankar, S., Shay, G., Jurczyluk, J., Ali, N., Down, J., Pocock, N., . . . Rogers, M. J. (2015). |
| | Real-time intravital imaging establishes tumor-associated macrophages as the extraskeletal |
| | target of bisphosphonate action in cancer. Cancer Discovery, 5(1), 35-42. |
| | http://doi.org/10.1158/2159-8290.CD-14-0621 |
| 80. | Huang, Z., Yang, Y., Jiang, Y., Shao, J., Sun, X., Chen, J., . . . Zhang, J. (2013). Anti-tumor |
| | immune responses of tumor-associated macrophages via toll-like receptor 4 triggered by |
| | cationic polymers. Biomaterials, 34(3), 746-55. |
| | http://doi.org/10.1016/j.biomaterials.2012.09.062 |
| 81. | Q. He, T. Fornander, H. Johansson et al., “Thymidine kinase 1 in serum predicts increased risk |
| | of distant or loco-regional recurrence following surgery in patients with early breast cancer,” |
| | Anticancer Research, vol. 26, no. 6, pp. 4753-4759, 2006. |
| 82. | K. L. O'Neill, M. Hoper, and G. W. Odling-Smee, “Can thymidine kinase levels in breast tumors |
| | predict disease recurrence?” Journal of the National Cancer Institute, vol. 84, no. 23, pp. 1825- |
| | 1828, 1992. |

What is claimed is:

1. A nucleic acid delivery system, comprising:
a delivery vector, the delivery vector comprising:
a nucleic acid comprising a polynucleotide encoding:
a chimeric receptor, the chimeric receptor comprising:
a cytoplasmic domain;
a transmembrane domain; and
an extracellular domain; and
wherein the extracellular domain of the chimeric receptor is other than any wild-type extracellular domain found in any wild-type protein comprising the same cytoplasmic portion as the chimeric receptor; and
a nucleic acid inhibitor that decreases expression of a protein that targets the cytoplasmic domain for degradation.

2. The nucleic acid delivery system of claim 1, further comprising a targeting agent, wherein the targeting agent directs the delivery vector to a cell.

3. The nucleic acid delivery system of claims 1 and 2, wherein the targeting agent also acts as an activating agent for the cell.

4. The nucleic acid delivery system of any one of the preceding claims, wherein the cell is a leukocyte.

5. The nucleic acid delivery system of any one of the preceding claims, wherein the leukocyte is a macrophage or dendritic cell.

6. The nucleic acid delivery system of any one of the preceding claims, wherein the cell is in vivo.

7. The nucleic acid delivery system of any one of the preceding claims, wherein the cell is a macrophage is expressing the chimeric receptor.

8. The nucleic acid delivery system of any one of the preceding claims, wherein the nucleic acid is an RNA.

9. The nucleic acid delivery system of any one of the preceding claims, wherein activation of the cytoplasmic portion of the chimeric receptor polarizes the macrophage or dendritic cell to an M1 macrophage.

10. The nucleic acid delivery system of any one of the preceding claims, wherein activation of the cytoplasmic portion of the chimeric receptor polarizes the macrophage or dendritic cell to an M2 macrophage.

11. The nucleic acid delivery system of any one of the preceding claims, wherein the extracellular domain is an antibody.

12. The nucleic acid delivery system of any one of the preceding claims, wherein the protein is RFN216 or Rab7b.

13. The nucleic acid delivery system of any one of the preceding claims, wherein the nucleic acid inhibitor is an shRNA that is not integrated into the genome.

14. The nucleic acid delivery system of any one of the preceding claims, wherein the binding of a ligand to the extracellular domain of the chimeric receptor initiates downstream signaling in the cytoplasmic portion.

15. The nucleic acid delivery system of any one of the preceding claims, wherein the cytoplasmic portion of the chimeric receptor comprises a cytoplasmic domain from a toll-like receptor, myeloid differentiation primary response protein (MYD88), toll-like receptor 3 (TLR3), toll-like receptor 4 (TLR4), toll-like receptor 7 (TLR7), toll-like receptor 8 (TLR8), toll-like receptor 9 (TLR9), myelin and lymphocyte protein (MAL), interleukin-1 receptor associated kinase 1 (IRAK1), low affinity immunoglobulin gamma Fc region receptor III-A (FCGR3A), low affinity immunoglobulin gamma Fc region receptor II-a (FCGR2A), and high affinity immunoglobulin epsilon receptor subunit gamma (FCER1G).

16. The nucleic acid delivery system of any one of the preceding claims, wherein the ligand is selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), B7-H3, PSCA, PSMA, CD19, CD20, CD30, CD33, CD123, CD133, CD138, and CD171.

17. The nucleic acid delivery system of any one of the preceding claims, wherein the extracellular domain of the chimeric receptor is an antibody or fragment thereof specific for a ligand selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.

18. The nucleic acid delivery system of any one of the preceding claims, wherein the antibody or fragment thereof is a single-chain variable fragment (scFv) or a single-domain antibody (sdAb).

19. The nucleic acid delivery system of any one of the preceding claims, wherein the chimeric receptor further comprises a hinge between the transmembrane domain and the extracellular domain.

20. The nucleic acid delivery system of any one of the preceding claims, wherein the targeting agent is protein based.

21. The nucleic acid delivery system of any one of the preceding claims, wherein the targeting agent is lipid based.

22. The nucleic acid delivery system of any one of the preceding claims, wherein activation of the cytoplasmic portion of the chimeric receptor polarizes the macrophage.

23. The nucleic acid delivery system of any one of the preceding claims, wherein the nucleic acid comprises a promoter operably linked to the polynucleotide.

24. The nucleic acid delivery system of any one of the preceding claims, wherein the promoter is selected from the group consisting of CD68 promoter (for example CD68/150(r)), EF1α, CMV, lysM, csflr, CD11c, SRA, and CDF11b.

25. The nucleic acid delivery system of any one of the preceding claims, wherein the delivery vector is a liposome.

26. The nucleic acid delivery system of any one of the preceding claims, wherein the delivery vector is a lipid nanoparticle.

27. The nucleic acid delivery system of any one of the preceding claims, wherein the targeting agent is a macrophage ligand.

28. The nucleic acid delivery system of any one of the preceding claims, wherein the macrophage ligand is selected from the group consisting of toll-like receptor, GM-CSF, CD14, CD16, CD64, CD115, CD192, CX2CR1, CD226, CD284, CD155, or any combination thereof comprises anyone of CD3, CD4, CD8, CD23, or CD28.

29. A cell expressing the chimeric receptor and the nucleic acid inhibitor of claim 1, wherein the cell is a leukocyte.

30. The cell of claim 29, wherein the leukocyte is a macrophage or dendritic cell.

31. The cell of any one of claims 29 to 30, wherein activation of the cytoplasmic portion of the chimeric receptor polarizes a macrophage to a M1 macrophage.

32. The leukocyte of any one of claims 29 to 31, wherein activation of the cytoplasmic portion of the chimeric receptor polarizes a macrophage to a M2 macrophage.

33. A delivery system for a chimeric antigen receptor, the system comprising:
a shell encasing mRNA encoding a chimeric receptor;
the chimeric receptor comprising:
a cytoplasmic domain;
a transmembrane domain; and
an extracellular domain; and
wherein a wild-type protein comprising the cytoplasmic portion does not comprise the extracellular domain.

34. A method of providing a nucleic acid to a cell, the method comprising:
providing the delivery vector of claim 1 or the delivery system of claim 33 to a patient; and
delivering the nucleic acid into the cell.

35. The method of claim 34, further comprising a targeting agent wherein the targeting agent directs the delivery vector to the cell.

36. The method of claims 34 and 35, wherein the targeting agent also acts as an activating agent for the cell.

37. The method of any one of claims 34 to 36, wherein the cell is a leukocyte.

38. The method of any one of claims 34 to 37, wherein the leukocyte is a macrophage or dendritic cell.

39. The method of any one of claims 34 to 38, wherein the cell is in vivo.

40. The method of any one of claims 34 to 39, wherein the macrophage is expressing the chimeric receptor.

41. The method of any one of claims 34 to 40, wherein the nucleic acid is an RNA.

42. The method of any one of claims 34 to 41, further comprising: providing a ligand to the extracellular domain of the chimeric receptor.

43. The method of any one of claims 34 to 42, further comprising: binding the ligand to the extracellular domain of the chimeric receptor.

44. The method of any one of claims 34 to 43, wherein the binding of the ligand to the extracellular domain of the chimeric receptor initiates downstream signaling in the cytoplasmic portion.

45. The method of any one of claims 34 to 44, wherein activation of the cytoplasmic portion polarizes the macrophage or dendritic cell.

46. The method of any one of claims 34 to 45, wherein activation of the cytoplasmic portion of the chimeric receptor polarizes a macrophage or dendritic cell to an M1 macrophage.

47. The method of any one of claims 34 to 46, wherein activation of the cytoplasmic portion of the chimeric receptor polarizes a macrophage or dendritic cell to an M2 macrophage.

48. The method of any one of claims 34 to 47, wherein the extracellular domain is an antibody.

49. The method of any one of claims 34 to 48, wherein the protein is RFN216 or Rab7b.

50. The method of any one of claims 34 to 49, wherein the nucleic acid inhibitor is an shRNA that is not integrated into the genome.

51. The method of any one of claims 34 to 50, wherein the cytoplasmic portion of the chimeric receptor comprises a cytoplasmic domain from a toll-like receptor, myeloid differentiation primary response protein (MYD88), toll-like receptor 3 (TLR3), toll-like receptor 4 (TLR4), toll-like receptor 7 (TLR7), toll-like receptor 8 (TLR8), toll-like receptor 9 (TLR9), myelin and lymphocyte protein (MAL), interleukin-1 receptor associated kinase 1 (IRAK1), low affinity immunoglobulin gamma Fc region receptor III-A (FCGR3A), low affinity immunoglobulin gamma Fc region receptor II-a (FCGR2A), and high affinity immunoglobulin epsilon receptor subunit gamma (FCER1G).

52. The method of any one of claims 34 to 51, wherein the ligand is selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.

53. The method of any one of claims 34 to 52, wherein the extracellular domain of the chimeric receptor is an antibody or fragment thereof specific for a ligand selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.

54. The method of any one of claims 34 to 53, wherein the antibody or fragment thereof is a single-chain variable fragment (scFv) or a single-domain antibody (sdAb).

55. The method of any one of claims 34 to 54, wherein the chimeric receptor further comprises a hinge between the transmembrane domain and the extracellular domain.

56. The method of any one of claims 34 to 55, wherein the targeting agent is protein based.

57. The method of any one of claims 34 to 56, wherein the targeting agent is lipid based.

58. The method of any one of claims 34 to 57, wherein activation of the cytoplasmic portion of the chimeric receptor polarizes the macrophage.

59. The method of any one of claims 34 to 58, wherein the nucleic acid comprises a promoter operably linked to the polynucleotide.

60. The method of any one of claims 34 to 59, wherein the promoter is selected from the group consisting of CD68 promoter (for example CD68/150(r)), EF1a, CMV, lysM, csflr, CD11c, SRA, and CDF11b.

61. The method of any one of claims 34 to 60, wherein the delivery vector is a liposome.

62. The method of any one of claims 34 to 61, wherein the delivery vector is a lipid nanoparticle.

63. The method of any one of claims 34 to 62, wherein the delivery vector has an outer shell comprising a lipid hydrophobic tail and a hydrophilic head.

64. The method of any one of claims 34 to 63, wherein the targeting agent is a macrophage ligand.

65. The method of any one of claims 34 to 64, wherein the macrophage ligand is selected from the group consisting of toll-like receptor, GM-CSF, CD14, CD16, CD64, CD115, CD192, CX2CR1, CD226, CD284, CD155, or any combination thereof comprises anyone of CD3, CD4, CD8, CD23, or CD28.

66. A method of providing a nucleic acid to a cell, the method comprising:
providing the delivery system of claim 33 to a patient; and
delivering the nucleic acid into the cell.

67. The method of claim 66, further comprising a targeting agent wherein the targeting agent directs the delivery vector to the cell.

68. The method of claims 66 and 67, wherein the targeting agent also acts as an activating agent for the cell.

69. The method of any one of claims 66 to 68, wherein the cell is a leukocyte.

70. The method of any one of claims 66 to 69, wherein the leukocyte is a macrophage or dendritic cell.

71. The method of any one of claims 66 to 70, wherein the cell is in vivo.

72. The method of any one of claims 66 to 71, wherein the macrophage is expressing the chimeric receptor.

73. The method of any one of claims 66 to 72, further comprising: providing a ligand to the extracellular domain of the chimeric receptor.

74. The method of any one of claims 66 to 73, further comprising: binding the ligand to the extracellular domain of the chimeric receptor.

75. The method of any one of claims 66 to 74, wherein the binding of the ligand to the extracellular domain of the chimeric receptor initiates downstream signaling in the cytoplasmic portion.

76. The method of any one of claims 66 to 75, wherein activation of the cytoplasmic portion polarizes the macrophage or dendritic cell.

77. The method of any one of claims 66 to 76, wherein activation of the cytoplasmic portion of the chimeric receptor polarizes a macrophage to an M1 macrophage.

78. The method of any one of claims 66 to 77, wherein activation of the cytoplasmic portion of the chimeric receptor polarizes a macrophage to an M2 macrophage.

79. The method of any one of claims 66 to 78, wherein the cytoplasmic portion of the chimeric receptor comprises a cytoplasmic domain from a toll-like receptor, myeloid differentiation primary response protein (MYD88), toll-like receptor 3 (TLR3), toll-like receptor 4 (TLR4), toll-like receptor 7 (TLR7), toll-like receptor 8 (TLR8), toll-like receptor 9 (TLR9), myelin and lymphocyte protein (MAL), interleukin-1 receptor associated kinase 1 (IRAK1), low affinity immunoglobulin gamma Fc region receptor III-A (FCGR3A), low affinity immunoglobulin gamma Fc region receptor II-a (FCGR2A), and high affinity immunoglobulin epsilon receptor subunit gamma (FCER1G).

80. The method of any one of claims 66 to 79, wherein the ligand is selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.

81. The method of any one of claims 66 to 80, wherein the extracellular domain of the chimeric receptor is an antibody or fragment thereof specific for a ligand selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.

82. The method of any one of claims 66 to 81, wherein the extracellular domain is an antibody.

83. The method of any one of claims 66 to 82, wherein the antibody or fragment thereof is a single-chain variable fragment (scFv) or a single-domain antibody (sdAb).

84. The method of any one of claims 66 to 83, wherein the targeting agent is protein based.

85. The method of any one of claims 66 to 84, wherein the targeting agent is lipid based.

86. The method of any one of claims 66 to 85, wherein the chimeric receptor further comprises a hinge region between the transmembrane domain and the linker.

87. The method of any one of claims 66 to 86, wherein the nucleic acid comprises a promoter operably linked to the polynucleotide.

88. The method of any one of claims 66 to 87, wherein the promoter is selected from the group consisting of CD68 promoter (for example CD68/150(r)), EF1a, CMV, lysM, csflr, CD11c, SRA, and CDF11b.

89. The method of any one of claims 66 to 88, wherein the shell is a liposome.

90. The method of any one of claims 66 to 89, wherein the shell is a lipid nanoparticle.

91. The method of any one of claims 66 to 90, wherein the targeting agent is a macrophage ligand.

92. The method of any one of claims 66 to 91, wherein the macrophage ligand is selected from the group consisting of toll-like receptor, GM-CSF, CD14, CD16, CD64, CD115, CD192, CX2CR1, CD226, CD284, CD155, or any combination thereof comprises anyone of CD3, CD4, CD8, CD23, or CD28.

93. The method of any one of claims 66 to 92, wherein activation of the cytoplasmic portion of the chimeric receptor polarizes the macrophage.

94. A method of producing a polarized macrophage in a subject, the method comprising:
administering to the subject a nanoparticle, the nanoparticle comprising a nucleic acid encoding a chimeric antigen receptor;
wherein the chimeric receptor comprises:
a cytoplasmic domain;
a transmembrane domain; and
an extracellular domain;
wherein the extracellular domain of the chimeric receptor is other than any wild-type extracellular domain found in any wild-type protein comprising the same cytoplasmic portion as the chimeric receptor;
wherein the extracellular domain specifically binds a target; and
wherein binding of the target by the extracellular domain activates the cytoplasmic domain to polarize a macrophage,
wherein the nanoparticle is taken up by a macrophage in the subject;
wherein the nucleic acid is expressed in the macrophage to produce the chimeric antigen receptor; and
wherein the extracellular domain binds specifically to its target in the subject and thereby signals the macrophage to become a polarized macrophage.

95. The method according to claim 94, wherein the nanoparticle further comprises a targeting agent, wherein the targeting agent directs uptake of the nanoparticle by the macrophage.

96. The method according to claim 94, wherein the nanoparticle further comprises a macrophage activating agent.

97. The method according to claim 97, wherein the activating agent and the targeting agent are the same.

98. The method according to claim 95, wherein the targeting agent is mannose.

99. The method according to claim 94, wherein the cytoplasmic domain comprises an antigen binding antibody fragment.

100. The method according to claim 99, wherein the cytoplasmic domain polarizes the macrophage to a M1 phenotype macrophage upon binding of the antibody fragment to its antigen.

101. The method according to claim 99, wherein the antibody fragment is a single chain variable fragment (scFv).

102. The method according to claim 101, wherein the scFv is derived from a monoclonal antibody specific for the antigen expressed by cells of a cancer.

103. The method according to claim 102, wherein the monoclonal antibody is a human or mouse monoclonal antibody.

104. The method according to claim 94, wherein the target is present on cells of a cancer.

105. The method according to claim 94, wherein the macrophage are present in a tumor.

106. The method according to claim 94, wherein the macrophage is a tumor associated macrophage.

107. A method of producing a polarized tumor associated macrophage (TAM) in a subject, the method comprising:
administering to the subject a nanoparticle, the nanoparticle comprising a nucleic acid encoding a chimeric antigen receptor;
wherein the chimeric receptor comprises:
a cytoplasmic domain;
a transmembrane domain; and
an extracellular domain;
wherein the extracellular domain of the chimeric receptor is other than any wild-type extracellular domain found in any wild-type protein comprising the same cytoplasmic portion as the chimeric receptor;
wherein the extracellular domain specifically binds a target; and
wherein binding of the target by the extracellular domain activates the cytoplasmic domain to polarize a TAM,
wherein the nanoparticle is taken up by the TAM in the subject;
wherein the nucleic acid is expressed in the TAM to produce the chimeric antigen receptor; and
wherein the extracellular domain binds specifically to its target in the subject and thereby signals the TAM to become a polarized TAM.

108. The method according to claim 107, wherein the nanoparticle further comprises a targeting agent, wherein the targeting agent directs uptake of the nanoparticle by the TAM.

109. The method according to claim 107, wherein the nanoparticle further comprises a TAM activating agent.

110. The method according to claim 109, wherein the activating agent and the targeting agent are the same.

111. The method according to claim 108, wherein the targeting agent is mannose.

112. The method according to claim 107, wherein the cytoplasmic domain comprises an antigen binding antibody fragment.

113. The method according to claim 112, wherein the cytoplasmic domain polarizes the TAM to a M1 phenotype TAM upon binding of the antibody fragment to its antigen.

114. The method according to claim 112, wherein the antibody fragment is a single chain variable fragment (scFv).

115. The method according to claim 114, wherein the scFv is derived from a monoclonal antibody specific for the antigen expressed by cells of a cancer.

116. The method according to claim 115, wherein the monoclonal antibody is a human or mouse monoclonal antibody.

117. The method according to claim 107, wherein the target is present on cells of a cancer.

118. The method according to claim 107, wherein the TAM are present in a tumor. (altering tumor)

119. The method according to claim 107, wherein the TAM are a tumor associated TAM.

120. A method of altering a tumor in a subject, the method comprising:
administering to the subject a nanoparticle, the nanoparticle comprising a nucleic acid encoding a chimeric antigen receptor;
wherein the chimeric receptor comprises:
a cytoplasmic domain;
a transmembrane domain; and
an extracellular domain;
wherein the extracellular domain of the chimeric receptor is other than any wild-type extracellular domain found in any wild-type protein comprising the same cytoplasmic portion as the chimeric receptor;
wherein the extracellular domain specifically binds a target; and
wherein binding of the target by the extracellular domain activates the cytoplasmic domain to polarize a macrophage,
wherein the nanoparticle is taken up by a macrophage in the subject;
wherein the nucleic acid is expressed in the macrophage to produce the chimeric antigen receptor;
wherein the extracellular domain binds specifically to its target in the subject and thereby signals the macrophage to become a polarized macrophage
wherein the macrophages are present in the tumor; and
wherein the macrophages are tumor associated macrophages.

121. The method according to claim 120, wherein the nanoparticle further comprises a targeting agent, wherein the targeting agent directs uptake of the nanoparticle by the macrophage.

122. The method according to claim 120, wherein the nanoparticle further comprises a macrophage activating agent.

123. The method according to claim 122, wherein the activating agent and the targeting agent are the same.

124. The method according to claim 121, wherein the targeting agent is mannose.

125. The method according to claim 120, wherein the cytoplasmic domain comprises an antigen binding antibody fragment.

126. The method according to claim 125, wherein the cytoplasmic domain polarizes the macrophage to a M1 phenotype macrophage upon binding of the antibody fragment to its antigen.

127. The method according to claim 125, wherein the antibody fragment is a single chain variable fragment (scFv).

128. The method according to claim 127, wherein the scFv is derived from a monoclonal antibody specific for the antigen expressed by cells of a cancer. (method treating cancer)

129. The method according to claim 128, wherein the monoclonal antibody is a human or mouse monoclonal antibody.

130. A method of treating a cancer in a subject, the method comprising:
administering to the subject a nanoparticle, the nanoparticle comprising a nucleic acid encoding a chimeric antigen receptor;
wherein the chimeric receptor comprises:
a cytoplasmic domain;
a transmembrane domain; and
an extracellular domain;
wherein the extracellular domain of the chimeric receptor is other than any wild-type extracellular domain found in any wild-type protein comprising the same cytoplasmic portion as the chimeric receptor;
wherein the extracellular domain specifically binds a target; and
wherein binding of the target by the extracellular domain activates the cytoplasmic domain to polarize a macrophage,
wherein the nanoparticle is taken up by a macrophage in the subject;
wherein the nucleic acid is expressed in the macrophage to produce the chimeric antigen receptor; and
wherein the extracellular domain binds specifically to the cancer cells in the subject and thereby signals the macrophage to become a polarized macrophage;
wherein the cytoplasmic domain comprises an antigen binding antibody fragment;
wherein the cytoplasmic domain polarizes the macrophage to an M1 phenotype macrophage upon binding of the antibody fragment to its antigen;
wherein the antibody fragment is a single chain variable fragment (scFV);
wherein the scFv is derived from a monoclonal antibody specific for the antigen expressed by cells of a cancer; and
wherein the target is present on the cancer cells.

131. The method according to claim 130, wherein the nanoparticle further comprises a targeting agent, wherein the targeting agent directs uptake of the nanoparticle by the macrophage.

132. The method according to claim 130, wherein the nanoparticle further comprises a macrophage activating agent.

133. The method according to claim 132, wherein the activating agent and the targeting agent are the same.

134. The method according to claim 131, wherein the targeting agent is mannose.

135. The method according to claim 130, wherein the monoclonal antibody is a human or mouse monoclonal antibody.

136. The method according to claim 130, wherein the macrophage are present in a tumor.

137. The method according to claim 130, wherein the macrophage are a tumor associated macrophage.


SEQUENCE LISTING (55 sequences)

SEQ ID NO: 1 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 2 - DNA, 2715 bp, Homo sapiens
atgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaactctttttcatgatcaataccagtatcctgttgatttttatctttattgtacttctcatccactttgagggctggaggatatctttttattggaatgtttcagtacatcgagttcttggtttcaaagaaatagacagacagacagaacagtttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcacttggatccaaaaactctgtacattaa

SEQ ID NO: 3 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 4 - protein, 23 aa, Homo sapiens
IGVSVLSVLVVSVVAVLVYKFYF

SEQ ID NO: 5 - protein, 183 aa, Homo sapiens
HLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 6 - DNA, 2520 bp, Homo sapiens
atgatgtctgcctcgcgcctggctgggactctgatcccagccatggccttcctctcctgcgtgagaccagaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctga

SEQ ID NO: 7 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 8 - DNA, 3150 bp, Homo sapiens
atggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctag

SEQ ID NO: 9 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 10 - DNA, 3126 bp, Homo sapiens
atggaaaacatgttccttcagtcgtcaatgctgacctgcattttcctgctaatatctggttcctgtgagttatgcgccgaagaaaatttttctagaagctatccttgtgatgagaaaaagcaaaatgactcagttattgcagagtgcagcaatcgtcgactacaggaagttccccaaacggtgggcaaatatgtgacagaactagacctgtctgataatttcatcacacacataacgaatgaatcatttcaagggctgcaaaatctcactaaaataaatctaaaccacaaccccaatgtacagcaccagaacggaaatcccggtatacaatcaaatggcttgaatatcacagacggggcattcctcaacctaaaaaacctaagggagttactgcttgaagacaaccagttaccccaaataccctctggtttgccagagtctttgacagaacttagtctaattcaaaacaatatatacaacataactaaagagggcatttcaagacttataaacttgaaaaatctctatttggcctggaactgctattttaacaaagtttgcgagaaaactaacatagaagatggagtatttgaaacgctgacaaatttggagttgctatcactatctttcaattctctttcacacgtgccacccaaactgccaagctccctacgcaaactttttctgagcaacacccagatcaaatacattagtgaagaagatttcaagggattgataaatttaacattactagatttaagcgggaactgtccgaggtgcttcaatgccccatttccatgcgtgccttgtgatggtggtgcttcaattaatatagatcgttttgcttttcaaaacttgacccaacttcgatacctaaacctctctagcacttccctcaggaagattaatgctgcctggtttaaaaatatgcctcatctgaaggtgctggatcttgaattcaactatttagtgggagaaatagcctctggggcatttttaacgatgctgccccgcttagaaatacttgacttgtcttttaactatataaaggggagttatccacagcatattaatatttccagaaacttctctaaacttttgtctctacgggcattgcatttaagaggttatgtgttccaggaactcagagaagatgatttccagcccctgatgcagcttccaaacttatcgactatcaacttgggtattaattttattaagcaaatcgatttcaaacttttccaaaatttctccaatctggaaattatttacttgtcagaaaacagaatatcaccgttggtaaaagatacccggcagagttatgcaaatagttcctcttttcaacgtcatatccggaaacgacgctcaacagattttgagtttgacccacattcgaacttttatcatttcacccgtcctttaataaagccacaatgtgctgcttatggaaaagccttagatttaagcctcaacagtattttcttcattgggccaaaccaatttgaaaatcttcctgacattgcctgtttaaatctgtctgcaaatagcaatgctcaagtgttaagtggaactgaattttcagccattcctcatgtcaaatatttggatttgacaaacaatagactagactttgataatgctagtgctcttactgaattgtccgacttggaagttctagatctcagctataattcacactatttcagaatagcaggcgtaacacatcatctagaatttattcaaaatttcacaaatctaaaagttttaaacttgagccacaacaacatttatactttaacagataagtataacctggaaagcaagtccctggtagaattagttttcagtggcaatcgccttgacattttgtggaatgatgatgacaacaggtatatctccattttcaaaggtctcaagaatctgacacgtctggatttatcccttaataggctgaagcacatcccaaatgaagcattccttaatttgccagcgagtctcactgaactacatataaatgataatatgttaaagttttttaactggacattactccagcagtttcctcgtctcgagttgcttgacttacgtggaaacaaactactctttttaactgatagcctatctgactttacatcttcccttcggacactgctgctgagtcataacaggatttcccacctaccctctggctttctttctgaagtcagtagtctgaagcacctcgatttaagttccaatctgctaaaaacaatcaacaaatccgcacttgaaactaagaccaccaccaaattatctatgttggaactacacggaaacccctttgaatgcacctgtgacattggagatttccgaagatggatggatgaacatctgaatgtcaaaattcccagactggtagatgtcatttgtgccagtcctggggatcaaagagggaagagtattgtgagtctggagctaacaacttgtgtttcagatgtcactgcagtgatattatttttcttcacgttctttatcaccaccatggttatgttggctgccctggctcaccatttgttttactgggatgtttggtttatatataatgtgtgtttagctaaggtaaaaggctacaggtctctttccacatcccaaactttctatgatgcttacatttcttatgacaccaaagatgcctctgttactgactgggtgataaatgagctgcgctaccaccttgaagagagccgagacaaaaacgttctcctttgtctagaggagagggattgggacccgggattggccatcatcgacaacctcatgcagagcatcaaccaaagcaagaaaacagtatttgttttaaccaaaaaatatgcaaaaagctggaactttaaaacagctttttacttggctttgcagaggctaatggatgagaacatggatgtgattatatttatcctgctggagccagtgttacagcattctcagtatttgaggctacggcagcggatctgtaagagctccatcctccagtggcctgacaacccgaaggcagaaggcttgttttggcaaactctgagaaatgtggtcttgactgaaaatgattcacggtataacaatatgtatgtcgattccattaagcaatactaa

SEQ ID NO: 11 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 12 - DNA, 3099 bp, Homo sapiens
atgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccacaataagctggacctctaccacgagcactcattcacggagctaccgcgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccagctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatag

SEQ ID NO: 13 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 14 - DNA, 954 bp, Homo sapiens
atgactatggagacccaaatgtctcagaatgtatgtcccagaaacctgtggctgcttcaaccattgacagttttgctgctgctggcttctgcagacagtcaagctgcagctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 15 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 16 - DNA, 765 bp, Homo sapiens
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 17 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 18 - DNA, 261 bp, Homo sapiens
atgattccagcagtggtcttgctcttactccttttggttgaacaagcagcggccctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacagtag

SEQ ID NO: 19 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 20 - DNA, 954 bp, Homo sapiens
atgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggttggctagaaggccacggggtgggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctga

SEQ ID NO: 21 - protein, 153 aa, Homo sapiens
MAPAAATGGSTLPSGFSVFTTLPDLLFIFEFIFGGLVWILVASSLVPWPLVQGWVMFVSVFCFVATTTLIILYIIGAHGGETSWVTLDAAYHCTAALFYLSASVLEALATITMQDGFTYRHYHENIAAVVFSYIATLLYVVHAVFSLIRWKSS

SEQ ID NO: 22 - DNA, 462 bp, Homo sapiens
atggcccccgcagcggcgacggggggcagcaccctgcccagtggcttctcggtcttcaccaccttgcccgacttgctcttcatctttgagtttatcttcgggggcctggtgtggatcctggtggcctcctccctggtgccctggcccctggtccagggctgggtgatgttcgtgtctgtgttctgcttcgtggccaccaccaccttgatcatcctgtacataattggagcccacggtggagagacttcctgggtcaccttggacgcagcctaccactgcaccgctgccctcttttacctcagcgcctcagtcctggaggccctggccaccatcacgatgcaagacggcttcacctacaggcactaccatgaaaacattgctgccgtggtgttctcctacatagccactctgctctacgtggtccatgcggtgttctctttaatcagatggaagtcttcataa

SEQ ID NO: 23 - protein, 712 aa, Homo sapiens
MAGGPGPGEPAAPGAQHFLYEVPPWVMCRFYKVMDALEPADWCQFAALIVRDQTELRLCERSGQRTASVLWPWINRNARVADLVHILTHLQLLRARDIITAWHPPAPLPSPGTTAPRPSSIPAPAEAEAWSPRKLPSSASTFLSPAFPGSQTHSGPELGLVPSPASLWPPPPSPAPSSTKPGPESSVSLLQGARPFPFCWPLCEISRGTHNFSEELKIGEGGFGCVYRAVMRNTVYAVKRLKENADLEWTAVKQSFLTEVEQLSRFRHPNIVDFAGYCAQNGFYCLVYGFLPNGSLEDRLHCQTQACPPLSWPQRLDILLGTARAIQFLHQDSPSLIHGDIKSSNVLLDERLTPKLGDFGLARFSRFAGSSPSQSSMVARTQTVRGTLAYLPEEYIKTGRLAVDTDTFSFGVVVLETLAGQRAVKTHGARTKYLKDLVEEEAEEAGVALRSTQSTLQAGLAADAWAAPIAMQIYKKHLDPRPGPCPPELGLGLGQLACCCLHRRAKRRPPMTQVYERLEKLQAVVAGVPGHSEAASCIPPSPQENSYVSSTGRAHSGAAPWQPLAAPSGASAQAAEQLQRGPNQPVESDESLGGLSAALRSWHLTPSCPLDPAPLREAGCPQGDTAGESSWGSGPGSRPTAVEGLALGSSASSSSEPPQIIINPARQKMVQKLALYEDGALDSLQLLSSSSLPGLGLEQDRQGPEESDEFQS

SEQ ID NO: 24 - DNA, 2139 bp, Homo sapiens
atggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctga

SEQ ID NO: 25 - protein, 257 aa, Artificial Sequence
  ScFv fragment
MSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDL

SEQ ID NO: 26 - protein, 5 aa, Artificial Sequence
  Short hinge linker
MNKTI

SEQ ID NO: 27 - protein, 55 aa, Artificial Sequence
  Long hinge linker
NDFACTCEHQSFLQWIKDQRQLLVEVERMECATPSDKQGMPVLSLNITCQMNKTI

SEQ ID NO: 28 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 12 aa, Homo sapiens
ESKYGPPCPPCP

SEQ ID NO: 30 - protein, 119 aa, Homo sapiens
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 228 aa, Homo sapiens
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 33 - protein, 68 aa, Artificial Sequence
  Mutated CD8 hinge
SALSNSIMYFSHFVPVFLPAKPTTTPAPRPPTPAPTIASQPLSLRPEASRPAAGGAVHTRGLDFASDI

SEQ ID NO: 34 - protein, 15 aa, Artificial Sequence
  GS linker
GGGGSGGGGSGGGGS

SEQ ID NO: 35 - protein, 496 aa, Artificial Sequence
  Chimeric antigen receptor
  SIGNAL (1)..(18)
  MISC_FEATURE (19)..(275): TK1-ScFv
  MISC_FEATURE (276)..(290): GS-Linker
  MISC_FEATURE (291)..(313): TLR4-Transmembrane domain
  MISC_FEATURE (314)..(496): TLR4-Cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 36 - protein, 500 aa, Artificial Sequence
  Chimeric antigen receptor
  SIGNAL (1)..(18)
  MISC_FEATURE (19)..(275): TK1-ScFv
  MISC_FEATURE (276)..(290): GS-Linker
  MISC_FEATURE (291)..(295): LRR Short Hinge
  MISC_FEATURE (296)..(318): TLR4 transmembrane domain
  MISC_FEATURE (319)..(500): TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATS

SEQ ID NO: 37 - protein, 551 aa, Artificial Sequence
  Chimeric antigen receptor
  SIGNAL (1)..(18): Singal peptide
  MISC_FEATURE (19)..(275): TK1-ScFv
  MISC_FEATURE (269)..(551): TLR4 cytosolic domain
  MISC_FEATURE (276)..(290): GS-Linker
  MISC_FEATURE (291)..(345): LRR long hinge
  MISC_FEATURE (346)..(368): TLR4 transmembrane domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 38 - protein, 508 aa, Artificial Sequence
  Chimeric antigen receptor
  SIGNAL (1)..(18)
  MISC_FEATURE (19)..(275): TK1 ScFv
  MISC_FEATURE (276)..(290): GS linker
  MISC_FEATURE (291)..(302): IgG4 short hinge
  MISC_FEATURE (303)..(325): TLR4 transmembrane domain
  MISC_FEATURE (326)..(508): TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 39 - protein, 615 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(409): IgG4 medium hinge
  MISC_FEATURE (410)..(432): TLR4 transmembrane domain
  MISC_FEATURE (433)..(615): TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 40 - protein, 724 aa, Artificial Sequence
  Chimeric antigen receptor
  SIGNAL (1)..(18)
  MISC_FEATURE (19)..(275): TK1 ScFv
  MISC_FEATURE (276)..(290): GS linker
  MISC_FEATURE (291)..(518): IgG4 long hinge
  MISC_FEATURE (519)..(541): TLR4 transmembrane domain
  MISC_FEATURE (542)..(724): TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 41 - protein, 564 aa, Artificial Sequence
  Chimeric antigen receptor
  SIGNAL (1)..(18): Signal peptide
  MISC_FEATURE (19)..(275)
  MISC_FEATURE (276)..(290): GS linkder
  MISC_FEATURE (291)..(358): Mutated CD8 hinge
  MISC_FEATURE (359)..(381): TLR4 transmembrane domain
  MISC_FEATURE (382)..(564): TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEASRPAAGGAVHTRGLDFASDIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 42 - protein, 564 aa, Artificial Sequence
  Chimeric antigen receptor
  SIGNAL (1)..(18): Sginal peptide
  MISC_FEATURE (19)..(275): TK1 ScFv
  MISC_FEATURE (276)..(290): GS linker
  MISC_FEATURE (291)..(358): GS linker
  MISC_FEATURE (359)..(381): TLR4 transmembrane domain
  MISC_FEATURE (382)..(564): TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEACRPAAGGAVHTRGLDFACDIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 43 - protein, 378 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(311): FCGR3A transmembrane domain
  MISC_FEATURE (312)..(336): FCGR3A cytosolic domain
  MISC_FEATURE (337)..(378): FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 44 - protein, 446 aa, Artificial Sequence
  Chimeric antigen receptor
  SIGNAL (1)..(18): Signal peptide
  MISC_FEATURE (19)..(275)
  MISC_FEATURE (276)..(290): GS linker
  MISC_FEATURE (291)..(358): Modified CD8a linker
  MISC_FEATURE (359)..(379): FCGR3A transmembrane domain
  MISC_FEATURE (380)..(404): FCGR3A cytosolic domain
  MISC_FEATURE (405)..(446): FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEASRPAAGGAVHTRGLDFASDIVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 45 - protein, 446 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(358): Portion of CD8 hinge
  MISC_FEATURE (359)..(379): FCGR3A transmembrane domain
  MISC_FEATURE (380)..(404): FCGR3A cytosolic domain
  MISC_FEATURE (405)..(446): FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEACRPAAGGAVHTRGLDFACDIVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 46 - protein, 391 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(303): IgG4 short hinge
  MISC_FEATURE (304)..(324): FCGR3A transmembrane domain
  MISC_FEATURE (325)..(349): FCGR3A cytosolic domain
  MISC_FEATURE (350)..(391): FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPIVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 47 - protein, 497 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linkder
  MISC_FEATURE (291)..(409): IgG4 119 aa hinge
  MISC_FEATURE (410)..(430): FCGR3A transmembrane domain
  MISC_FEATURE (431)..(455): FCGR3A cytosolic domain
  MISC_FEATURE (456)..(497): FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 48 - protein, 607 aa, Artificial Sequence
  Chimeric angtigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(519): IgG4 long hinge
  MISC_FEATURE (520)..(540): FCGR3A transmembrane domain
  MISC_FEATURE (541)..(565): FCGR3A cytosolic domain
  MISC_FEATURE (566)..(607): FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 49 - protein, 390 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(312): FCGR2A transmembrane domain
  MISC_FEATURE (313)..(390): FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 50 - protein, 458 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(358): Modified CD8a hinge
  MISC_FEATURE (359)..(380): FCGR2A transmembrane domain
  MISC_FEATURE (381)..(458): FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEASRPAAGGAVHTRGLDFASDIIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 51 - protein, 458 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(358): Portion of CD8a linker
  MISC_FEATURE (359)..(380): FCGR2A transmembrane domain
  MISC_FEATURE (381)..(458): FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEACRPAAGGAVHTRGLDFACDIIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 52 - protein, 403 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(303): IgG4 short hinge
  MISC_FEATURE (304)..(325): FCGR2A transmembrane domain
  MISC_FEATURE (326)..(403): FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPIIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 53 - protein, 509 aa, Artificial Sequence
  Chimeric antigen receptor
  SIGNAL (1)..(18): Signal peptide
  MISC_FEATURE (19)..(275): TK1 ScFv
  MISC_FEATURE (276)..(290): GS linker
  MISC_FEATURE (291)..(409): IgG4 119 aa hinge
  MISC_FEATURE (410)..(431): FCGR2A transmembrane domain
  MISC_FEATURE (432)..(509): FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 54 - protein, 619 aa, Artificial Sequence
  Chimeric antigen receptor
  SIGNAL (1)..(18): Signal peptide
  MISC_FEATURE (19)..(275): TK ScFv
  MISC_FEATURE (276)..(290): GS linker
  MISC_FEATURE (291)..(519): IgG4 long hinge
  MISC_FEATURE (520)..(541): FCGR2A transmembrane domain
  MISC_FEATURE (542)..(619): FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 55 - DNA, 5065 bp, Artificial Sequence
  Chimeric antigen receptor
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgccatcatgaagtttaaacaagcttgaattcgccaccatgacccggctgacagtcctggccctgctggctggtctgctggcgtcctcgagggcccaagtccagctccagcagtcgggcccagagttggagaagcctggggcgagcgtgaagatctcatgcaaagcctcaggctactcctttactggatacacgatgaattgggtgaaacagtcgcatggaaagtcactggaatggatcggtctgattacgccctacaacggcgcctccagctacaaccagaagttcaggggaaaggcgacccttactgtagacaagtcgtcaagcaccgcctacatggacctcctgtccctgacctccgaagatagcgcggtctacttttgtgcacgcggaggttacgatggacggggattcgactactggggccagggaaccactgtcaccgtgtcgagcggaggcggagggagcggaggaggaggcagcggaggtggagggtcggatatcgaactcactcagtccccagcaatcatgtccgcttcaccgggagaaaaggtgaccatgacttgctcggcctcctcgtccgtgtcatacatgcactggtaccaacaaaaatcggggacctcccctaagagatggatctacgataccagcaaactggcttcaggcgtgccgggacgcttctcgggttcggggagcggaaattcgtattcgttgaccatttcgtccgtggaagccgaggacgacgcaacttattactgccaacagtggtcaggctacccgctcactttcggagccggcactaagctggagatcaccggtgagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatggtcgacctggccctggggattctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggcctggatccgccacaaacttctctctgctaaagcaagcaggtgatgttgaagaaaaccccgggcctatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaagcggccgcgtttaaacagagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccacgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggttcgtctcgtcccggtctcctcccatgcatg